(19) United States
(12) Patent Application Publication
(10) Pub. No.: US 2012/0171161 A1
(43) Pub. Date: Jul. 5, 2012

(54) COMPOSITIONS COMPRISING PLACENTAL STEM CELLS AND PLATELET RICH PLASMA, AND METHODS OF USE THEREOF

(76) Inventors: Sascha Abramson, Holland Township, NJ (US); Mohit B. Bhatia, Manalapan, NJ (US); Uri Herzberg, Bridgewater, NJ (US)

(21) Appl. No.: 13/340,557

(22) Filed: Dec. 29, 2011

Related U.S. Application Data
(60) Provisional application No. 61/428,721, filed on Dec. 30, 2010.

(51) Int. Cl. A61K 35/50; A61P 9/10; A61P 37/08; A61P 11/06; A61P 3/10; A61P 25/00; A61P 1/00; A61P 19/02; A61P 17/06; A61K 35/16; A61P 37/06

(52) U.S. Cl. 424/93.3; 424/93.72

(57) ABSTRACT

Provided herein are compositions comprising placental stem cells and platelet rich plasma. Also provided herein are methods of treating an individual suffering from a disease or condition that would benefit from reduced inflammation, promotion of angiogenesis, and enhanced healing, comprising administering a therapeutically effective amount of a composition comprising placental stem cells and platelet rich plasma, as described herein, to said individual in an amount and for a time sufficient for detectable improvement of said disease or condition.

This application claims benefit of U.S. Provisional Patent Application No. 61/428,721, filed Dec. 30, 2010, the disclosure of which is hereby incorporated by reference in its entirety.

### 1. FIELD

Provided herein are compositions comprising placental stem cells, referred to herein as PDACs, and platelet rich plasma (PRP). Also provided herein are methods of treating an individual suffering from a disease or condition that would benefit from reduced inflammation, modulation of immune response, promotion of angiogenesis, and enhanced healing, comprising administering a therapeutically effective amount of a composition comprising PDACs and platelet rich plasma, as described herein, to said individual in an amount and for a time sufficient for detectable improvement of said disease or condition, e.g., a vascular condition, a non-healing or slow-healing wound, neuropathic pain, or an orthopedic defect, e.g., a spinal disc defect and arthritic joints.

### 2. BACKGROUND

Vascular conditions, non-healing or slow-healing wounds, neuropathic pain, and orthopedic defects, e.g., a spinal disc defects, among other conditions, continue to be important medical problems. There is a need for improved therapeutics for such conditions.

### 3. SUMMARY

Provided herein are compositions comprising placental stem cells (PDACs) or culture medium conditioned by PDACs, and platelet rich plasma (PRP), e.g., for use in treating a disease, disorder or medical condition in an individual. In some embodiments, administration of the compositions to an individual in need thereof results in prolonged localization of the placental stem cells at the site of injection or implantation, relative to administration of placental stem cells not combined with platelet rich plasma.

In some embodiments, said composition is suitable for injection into an individual, and wherein said placental stem cells are adherent to tissue culture plastic, are CD34⁻, CD10⁺, CD105⁺ and CD200⁺, and are not trophoblasts.

In some embodiments, said placental stem cells are not obtained from umbilical cord.

In some embodiments, said composition does not comprise an implantable bone substitute. In some embodiments, said composition does not require thrombin to retain said placental stem cells at a site of injection of said individual.

In a specific embodiment, said placental stem cells are CD10⁺, CD34⁻, CD105⁺, CD200⁺ placental stem cells. In another specific embodiment, said placental stem cells express CD200 and do not express HLA-G; or express CD73, CD105, and CD200; or express CD200 and OCT-4; or express CD73 and CD105 and do not express HLA-G; or express CD73 and CD105 and facilitate the formation of one or more embryoid-like bodies in a population of placental cells comprising said stem cell when said population is cultured under conditions that allow for the formation of an embryoid-like body; or express OCT-4 and facilitate the formation of one or more embryoid-like bodies in a population of placental cells comprising said stem cell when said population is cultured under conditions that allow for the formation of an embryoid-like body. In yet other embodiments, said placental stem cells express one or more of CD44, CD90, HLA-A,B,C, or ABC-p, and/or do not express one or more of CD45, CD117, CD133, KDR, CD80, CD86, HLH-DR, SSEA3, SSE4, or CD38. In certain embodiments, the placental stem cells suppress the activity of an immune cell, e.g., suppress proliferation of a T cell.

In some embodiments, said composition comprising PDACs and PRP is formulated to be administered to said individual by injection, e.g., local injection.

In some embodiments, the platelet rich plasma of the compositions provided herein is autologous platelet rich plasma. In some embodiments, the platelet rich plasma is derived from placental perfusate. In some embodiments, the platelet rich plasma is derived from a suitable donor.

In some embodiments, the volume to volume ratio of placental stem cells to platelet rich plasma in the composition is between about 10:1 and 1:10. In some embodiments, the volume to volume ratio of placental stem cells to platelet rich plasma in the composition is about 1:1. In some embodiments, the ratio of the number of placental stem cells to the number of platelets in the platelet rich plasma is between about 100:1 and 1:100. In some embodiments, the ratio of the number of placental stem cells to the number of platelets in the platelet rich plasma is about 1:1.

In another aspect, provided herein is a method of transplantation comprising administering to an individual, e.g., injecting an individual with, a composition comprising placental stem cells and platelet rich plasma, wherein said transplantation results in prolonged localization of said placental stem cells at the site or region of injection, relative to injection of placental stem cells not combined with platelet rich plasma.

In some embodiments, the placental stem cells and platelet rich plasma are combined to form said composition ex vivo prior to said injecting the individual. In some embodiments, the platelet rich plasma is injected into the individual in a first step, and the placental stem cells are injected into or near the site of platelet rich plasma injection in a second step, and the composition is formed in vivo.

In some embodiments of the methods of transplantation provided herein, the volume to volume ratio of placental stem cells to platelet rich plasma in the composition is between about 10:1 and 1:10. In some embodiments, the volume to volume ratio of placental stem cells to platelet rich plasma in the composition is about 1:1. In some embodiments, the ratio of the number of placental stem cells to the number of platelets in the platelet rich plasma is between about 100:1 and 1:100. In some embodiments, the ratio of the number of placental stem cells to the number of platelets in the platelet rich plasma is about 1:1.

In another aspect, provided herein is a method of treating an individual having or at risk of developing critical limb ischemia, comprising administering to the individual a therapeutically effective amount of a composition comprising placental stem cells and platelet rich plasma.

In another aspect, provided herein is a method of treating an individual having leg ulcer, comprising contacting the leg ulcer with a therapeutically effective amount of a composition comprising placental stem cells and platelet rich plasma. In some embodiments, the leg ulcer is a venous leg ulcer, arterial leg ulcer, diabetic leg ulcer, decubitus ulcer, or split thickness skin grafted ulcer.

In another aspect, provided herein is a method of treating an individual having degenerative disc disorder, comprising administering to the individual a therapeutically effective amount of a composition comprising placental stem cells and platelet rich plasma.

In another aspect, provided herein is a method of treating an individual having a herniated disc, comprising contacting the herniated disc with a therapeutically effective amount of a composition comprising placental stem cells and platelet rich plasma.

In another aspect, provided herein is a method of treating an individual having neuropathic pain, comprising administering to the individual a therapeutically effective amount of a composition comprising placental stem cells and platelet rich plasma.

### 3.1 Definitions

As used herein, the term “about,” when referring to a stated numeric value, indicates a value within plus or minus 10% of the stated numeric value.

As used herein, the term “amount,” when referring to the placental stem cells described herein, means a particular number of placental cells.

As used herein, the term “stem cell” defines a cell that retains at least one attribute of a stem cell, e.g., a marker or gene expression profile associated with one or more types of stem cells; the ability to replicate at least 10-40 times in culture; multipotency, e.g., the ability to differentiate, either in vitro, in vivo or both, into cells of one or more of the three germ layers; the lack of adult (i.e., differentiated) cell characteristics, or the like.

As used herein, the term “derived” means isolated from or otherwise purified. For example, placental derived adherent cells are isolated from placenta. The term “derived” encompasses cells that are cultured from cells isolated directly from a tissue, e.g., the placenta, and cells cultured or expanded from primary isolates.

As used herein, “immunolocalization” means the detection of a compound, e.g., a cellular marker, using an immune protein, e.g., an antibody or fragment thereof in, for example, flow cytometry, fluorescence-activated cell sorting, magnetic cell sorting, in situ hybridization, immunohistochemistry, or the like.

As used herein, the term “SH2” refers to an antibody that binds an epitope on the marker CD105. Thus, cells that are referred to as SH2⁺ are CD105⁺.

As used herein, the terms “SH3” and SI-14″ refer to antibodies that bind epitopes present on the marker CD73. Thus, cells that are referred to as SH3⁺ and/or SH4⁺ are CD73⁺.

As used herein, cells, e.g., PDACs are “isolated” if at least 50%, 60%, 70%, 80%, 90%, 95%, or at least 99% of other cells with which the stem cells are naturally associated are removed from the stem cells, e.g., during collection and/or culture of the stem cells.

As used herein, the term “isolated population of cells” means a population of cells that is substantially separated from other cells of the tissue, e.g., placenta, from which the population of cells is obtained or derived. In some embodiments, a population of, e.g., stem cells is “isolated” if at least 50%, 60%, 70%, 80%, 90%, 95%, or at least 99% of the cells with which the population of stem cells are naturally associated are removed from the population of stem cells, e.g., during collection and/or culture of the population of stem cells.

As used herein, the term “placental stem cell” refers to a stem cell or progenitor cell that is derived from, e.g., isolated from, a mammalian placenta, regardless of morphology, cell surface markers, or the number of passages after a primary culture, which adheres to a tissue culture substrate (e.g., tissue culture plastic or a fibronectin-coated tissue culture plate). The term “placenta stem cell” as used herein does not, however, refer to a trophoblast, a cytotrophoblast, embryonic germ cell, or embryonic stem cell, as those cells are understood by persons of skill in the art. The terms “placental stem cell” and “placenta-derived stem cell” may be used interchangeably. Unless otherwise noted herein, the term “placental” includes the umbilical cord. The placental stem cells disclosed herein are, in certain embodiments, multipotent in vitro (that is, the cells differentiate in vitro under differentiating conditions), multipotent in vivo (that is, the cells differentiate in vivo), or both.

As used herein, a stem cell is “positive” for a particular marker when that marker is detectable above background, e.g., by immunolocalization, e.g., by flow cytometry; or by RT-PCR, etc. For example, a cell or cell population is described as positive for, e.g., CD73 if CD73 is detectable on the cell, or in the cell population, in an amount detectably greater than background (in comparison to, e.g., an isotype control) or an experimental negative control for any given assay. In the context of, e.g., antibody-mediated detection, “positive,” as an indication a particular cell surface marker is present, means that the marker is detectable using an antibody, e.g., a fluorescently-labeled antibody, specific for that marker; “positive” also means that a cell or population of cells displays that marker in a amount that produces a signal, e.g., in a cytometer, ELISA, or the like, that is detectably above background. For example, a cell is “CD105⁺” where the cell is detectably labeled with an antibody specific to CD105, and the signal from the antibody is detectably higher than a control (e.g., background). Conversely, “negative” in the same context means that the cell surface marker is not detectable using an antibody specific for that marker compared to background. For example, a cell or population of cells is “CD34⁻” where the cell or population of cells is not detectably labeled with an antibody specific to CD34. Unless otherwise noted herein, cluster of differentiation (“CD”) markers are detected using antibodies. For example, OCT-4 can be determined to be present, and a cell is OCT-4⁺, if mRNA for OCT-4 is detectable using RT-PCR, e.g., for 30 cycles. A cell is also positive for a marker when that marker can be used to distinguish the cell from at least one other cell type, or can be used to select or isolate the cell when present or expressed by the cell.

As used herein, “immunomodulation” and “immunomodulatory” mean causing, or having the capacity to cause, a detectable change in an immune response, and the ability to cause a detectable change in an immune response, either systemically or locally.

As used herein, “immunosuppression” and “immunosuppressive” mean causing, or having the capacity to cause, a detectable reduction in an immune response, and the ability to cause a detectable suppression of an immune response, either systemically or locally.

### 4. DETAILED DESCRIPTION

### 4.1 PDACs and Platelet Rich Plasma

Provided herein are compositions comprising placental stem cells combined with platelet rich plasma, wherein administration of the compositions to an individual in need thereof results in prolonged localization of the placental stem cells at the site of injection or implantation, relative to administration of placental stem cells not combined with platelet rich plasma. In certain embodiments, the placental stem cells are human. In other embodiments, the platelet rich plasma is human, e.g., is obtained from or derived from a human source. In other embodiments, both the placental stem cells and PRP are human.

In various embodiments, the volume to volume ratio of placental stem cells to platelet rich plasma can be between about 10:1 and 1:10. In some embodiments, the volume to volume ratio of placental stem cells to platelet rich plasma is about 10:1, 9.5:1, 9:1, 8.5:1, 8:1, 7.5:1, 7:1, 6.5:1, 6:1, 5.5:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1.9.5, or 1:10. In particular embodiments, the volume to volume ratio of placental stem cells to platelet rich plasma is about 1:1. In some embodiments, the ratio of the number of placental stem cells to the number of platelets in the platelet rich plasma can be between about 100:1 and 1:100. In some embodiments, the volume to volume ratio of placental stem cells to platelet rich plasma is about 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10 1:15, 1:20, 1:25, 1:30, 1:35, 1:40, 1:45, 1:50, 1:55, 1:60, 1:65, 1:70, 1:75, 1:80, 1:85, 1:90, 1.95, or 1:100. In particular embodiments, the ratio of the number of placental stem cells to the number of platelets in the platelet rich plasma is about 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10 1:15, 1:20, 1:25, 1:30, 1:35, 1:40, 1:45, 1:50, 1:55, 1:60, 1:65, 1:70, 1:75, 1:80, 1:85, 1:90, 1.95, or 1:100.

The compositions comprising placental stem cells and platelet rich plasma provided herein can comprise a therapeutically-effective amount of placental stem cells, platelets, e.g., platelet rich plasma, or both. The combination compositions can comprise at least 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ placental stem cells, platelets in platelet rich plasma, or both, or no more than 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ placental stem cells, platelets in platelet rich plasma, or both.

In one embodiment, the individual is administered a dose of a combination composition comprising about 300 million placental stem cells. Dosage, however, can vary according to the individual's physical characteristics, e.g., weight, and can range from 1 million to 10 billion placental stem cells per dose, preferably between 10 million and 1 billion per dose, or between 100 million and 500 million placental stem cells per dose.

In other embodiments, transplantation of said composition comprising placental stem cells combined with platelet rich plasma prolongs localization of the placental stem cells at the site of injection or implantation at least, or at, 1, 2, 3, 4, 5, 6, 7, 8, 9, 10, 11, 12, 13, 14, 15, 16, 17, 18, 19, 20 or 21 days post-transplant, relative to transplantation of placental stem cells not combined with platelet rich plasma. In another more specific embodiment, said composition comprising placental stem cells combined with platelet rich plasma prolongs localization of the placental stem cells at the site of injection or implantation at least, or more than 21 days post-transplant. In specific embodiments, said composition comprising placental stem cells combined with platelet rich plasma prolongs localization of the placental stem cells at the site of injection or implantation at least, or more than 25, 30, 35, 40, 45, 50, 55 weeks, or 1 year or longer post-transplant.

### 4.2 Platelet Rich Plasma

The compositions and methods provided herein use placental stem cells in combination with platelet rich plasma (PRP). In some embodiments, the PRP useful in the combination compositions and methods provided herein comprises platelet cells at a concentration of at least 1.1-fold greater than the concentration of platelets in whole blood, e.g., unprocessed whole blood. In some embodiments, the PRP comprises platelet cells at a concentration of about 1.1-fold to about 10-fold greater than the concentration of platelets in whole blood. In some embodiments, the PRP comprises platelet cells at a concentration of about 1.5, 2.0, 2.5, 3.0, 3.5, 4, 4.5, 5, 5.5, 6, 6.5, 7, 7.5, 8, 8.5, 9, 9.5, 10-fold, or more than 10-fold greater than the concentration of platelets in whole blood.

Generally, a microliter of whole blood comprises between 140,000 and 500,000 platelets. In some embodiments, the platelet concentration in the PRP useful in the combination compositions and methods provided herein is between about 150,000 and about 2,000,000 platelets per microliter. In some embodiments, the platelet concentration in the PRP is about 150,000, 200,000, 300,000, 400,000, 500,000, 600,000, 700,000, 800,000, 900,000, 1,000,000, 1,100,000, 1,100,000, 1,200,000, 1,300,000, 1,400,000, 1,500,000, 1,600,000, 1,700,000, 1,800,000, 1,900,000, or 2,000,000 platelets per microliter. In some embodiments, the platelet concentration in the PRP is about 2,500,000 to about 5,000,000, or about 5,000,000 to about 7,000,000 platelets per microliter.

The combination compositions provided herein may comprise PRP derived from a human or animal source of whole blood. The PRP may be prepared from an autologous source, an allogeneic source, a single source, or a pooled source of platelets and/or plasma, e.g., platelets harvested from placental perfusate. The PRP can be isolated from whole blood or portions of whole blood using a variety of techniques comprising, for example, centrifugation, gravity filtration, and/or direct cell sorting.

PRP can be, e.g., prepared from a donor who has not been previously treated with a thrombolytic agent, such as heparin, tPA, or aspirin. In some embodiments, the donor has not received a thrombolytic agent for at least 2 hours, 1 day, 2 weeks, or 1 month prior to withdrawing the blood for extraction of the PRP.

To derive PRP from donor blood, whole blood may be collected from the donor, for example, using a blood collection syringe. The amount of blood collected may depend on a number of factors, including, for example, the amount of PRP desired, the health of the donor, the severity or location of the tissue damage in the individual to be treated, the availability of prepared PRP, or any suitable combination of factors.

Any suitable amount of blood may be collected. For example, about 30 to 60 ml of whole blood may be drawn. In an exemplary embodiment, about 11 ml of blood may be withdrawn into a syringe that contains about 5 ml of an anticoagulant, such as acid-citrate-phosphate or citrate-phosphate-dextrose solution. The syringe may be attached to an apheresis needle, and primed with the anticoagulant. Blood may be drawn from the donor using standard aseptic practice. In some embodiments, a local anesthetic such as anbesol, benzocaine, lidocaine, procaine, bupivicaine, or any appropriate anesthetic known in the art may be used to anesthetize the insertion area.

4.2.1 Methods of Obtaining Platelet Rich Plasma

Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette. Generally, at least 60% or at least 80% of the available platelets within the blood sample can be captured. These platelets may be resuspended in a volume that may be about 3% to about 20% or about 5% to about 10% of the sample volume. PRP may be isolated from whole blood by any method known in the art. For example, the PRP may be prepared from whole blood using a centrifuge. In a particular embodiment, whole blood is spun at 150-1350×g for 6 minutes at room temperature.

In another embodiment, whole blood can be centrifuged using a gravitational platelet system, such as the Cell Factor Technologies GPS SYSTEM™ centrifuge. The blood-filled syringe may be slowly transferred to a disposable separation tube which may be loaded into a port on the GPS centrifuge. The sample may be capped and placed into the centrifuge. The centrifuge may be counterbalanced with a tube comprising sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at 3200 rpm for extraction from a side of the separation tube and then isolated platelets may be suspended in about 3 cc to about 5 cc of plasma by agitation. The PRP may then be extracted from a side port using, for example, a 10 cc syringe. If about 55 cc of blood is collected from a patient, about 5 cc of PRP may be obtained.

The PRP may be buffered using an alkaline buffering agent to a physiological pH. The buffering agent may be a biocompatible buffer such as HEPES, TRIS, monobasic phosphate, monobasic bicarbonate, or any suitable combination thereof that may be capable of adjusting the PRP to physiological pH between about 6.5 and about 8.0. In certain embodiments, the physiological pH may be adjusted to about pH 7.3 to about pH 7.5, and more specifically, about pH 7.4. In certain embodiments, the buffering agent may be an 8.4% sodium bicarbonate solution. In this embodiment, for each cc of PRP isolated from whole blood, 0.05 cc of 8.4% sodium bicarbonate may be added. In some embodiments, the syringe may be gently shaken to mix the PRP and bicarbonate.

Platelet counts in the PRP can be counted and recorded, and the PRP can be resuspended for a precise number of wells in a compatible vehicle or in the donor's own plasma prior to combining with placental stem cells according to the methods described herein.

In some embodiments of the compositions and methods provided herein, the combination composition comprises placental stem cells and PRP derived from placental perfusate. An exemplary method for isolating PRP from placental perfusate is described below.

Following exsanguination of the umbilical cord and placenta, the placenta is placed in a sterile, insulated container at room temperature and delivered to the laboratory within 4 hours of birth. The placenta is discarded if, on inspection, it has evidence of physical damage such as fragmentation of the organ or avulsion of umbilical vessels. The placenta is maintained at room temperature (23°+/−2° C.) or refrigerated (4° C.) in sterile containers for 2 to 20 hours. Periodically, the placenta is immersed and washed in sterile saline at 25°+/−3° C. to remove any visible surface blood or debris. The umbilical cord is transected approximately 5 cm from its insertion into the placenta and the umbilical vessels are cannulated with Teflon or polypropylene catheters connected to a sterile fluid path allowing bidirectional perfusion of the placenta and recovery of the effluent fluid.

The placenta is maintained under conditions which simulate and sustain a physiologically compatible environment for the proliferation and recruitment of residual cells. The cannula is flushed with IMDM serum-free medium (GibcoBRL, NY) containing 2 U/ml heparin (Elkins-Sinn, N.J.). Perfusion of the placenta is performed at a rate of 50 mL per minute. During the course of the procedure, the placenta is gently massaged to aid in the perfusion process and assist in the recovery of cellular material. Effluent fluid is collected from the perfusion circuit by both gravity drainage and aspiration through the arterial cannula.

The perfusion and collection procedures may be repeated once or twice until the number of recovered nucleated cells falls below 100/microL. The perfusates are pooled and subjected to light centrifugation to isolate platelets. Platelets can be resuspended for a precise number of wells in a compatible vehicle or in the donor's own plasma prior to combining with placental stem cells according to the methods described herein.

### 4.3 Placental Stem Cells and Placental Stem Cell Populations

The compositions and methods provided herein use placental stem cells, that is, stem cells obtainable from a placenta or part thereof, that (1) adhere to a tissue culture substrate; (2) have the capacity to differentiate into non-placental cell types; and (3) preferably have, in sufficient numbers, the capacity to detectably suppress an immune function, e.g., proliferation of CD4⁺ and/or CD8⁺ T cells in an MLR assay or regression assay. The immunosuppressive properties of placental stem cells are described in U.S. Patent Application Publication Nos. 2007/0190034 and 2008/0226595, the disclosures of which are hereby incorporated by reference in their entireties. Placental stem cells are not derived from blood, e.g., placental blood or umbilical cord blood. The placental stem cells used in the compositions and methods provided herein preferably have the capacity, and can be selected for their capacity, to suppress the immune system of an individual.

Placental stem cells can be either fetal or maternal in origin (that is, can have the genotype of either the mother or fetus). Populations of placental stem cells, or populations of cells comprising placental stem cells, can comprise placental stem cells that are solely fetal or maternal in origin, or can comprise a mixed population of placental stem cells of both fetal and maternal origin. The placental stem cells, and populations of cells comprising the placental stem cells, can be identified and selected by the morphological, marker, and culture characteristics discussed below.

4.3.1 Physical and Morphological Characteristics

The placental stem cells used as described herein, when cultured in primary cultures or in cell culture, adhere to the tissue culture substrate, e.g., tissue culture container surface (e.g., tissue culture plastic). Placental stem cells in culture assume a generally fibroblastoid, stellate appearance, with a number of cyotplasmic processes extending from the central cell body. The placental stem cells are, however, morphologically differentiable from fibroblasts cultured under the same conditions, as the placental stem cells exhibit a greater number of such processes than do fibroblasts. Morphologically, placental stem cells are also differentiable from hematopoietic stem cells, which generally assume a more rounded, or cobblestone, morphology in culture.

4.3.2 Cell Surface, Molecular and Genetic Markers

Isolated placental stem cells and populations of such isolated placental stem cells, useful in the compositions and methods disclosed herein, are tissue culture plastic-adherent placental stem cells, and express a plurality of markers that can be used to identify and/or isolate the cells, or populations of cells that comprise the stem cells. In certain embodiments, the PDACs are angiogenic, e.g., in vitro or in vivo. The isolated placental stem cells, and placental stem cell populations described herein (that is, two or more isolated placental stem cells) include placental stem cells and placental stem cell-containing cell populations obtained directly from the placenta, or any part thereof (e.g., chorion, placental cotyledons, or the like), or derived from such cells. Isolated placental stem cell populations also include populations of (that is, two or more) isolated placental stem cells in culture, and a population in a container, e.g., a bag. The isolated placental stem cells described herein are not bone marrow-derived mesenchymal cells, adipose-derived mesenchymal stem cells, or mesenchymal cells obtained from umbilical cord blood, placental blood, or peripheral blood. Placental stem cells useful in the compositions and methods described herein are described herein and, e.g., in U.S. Pat. Nos. 7,311,904; 7,311,905; and 7,468,276; and in U.S. Patent Application Publication No. 2007/0275362, the disclosures of which are hereby incorporated by reference in their entireties.

In certain other embodiments, the isolated placental stem cells are multipotent cells. In one embodiment, the isolated placental stem cells, e.g, PDACs, are CD34⁻, CD10⁺ and CD105⁺ as detected by flow cytometry. In another specific embodiment, the isolated CD34⁻, CD10⁺, CD105⁺ placental stem cells have the potential to differentiate into cells of a neural phenotype, cells of an osteogenic phenotype, and/or cells of a chondrogenic phenotype. In another specific embodiment, the isolated CD34⁻, CD10⁺, CD105⁺ placental stem cells are additionally CD200⁺. In another specific embodiment, the isolated CD34⁻, CD10⁺, CD105⁺ placental stem cells are additionally CD45⁻ or CD90⁺. In another specific embodiment, the isolated CD34⁻, CD10⁺, CD105⁺ placental stem cells are additionally CD45⁻ and CD90⁺, as detected by flow cytometry. In another specific embodiment, the isolated CD34⁻, CD10⁺, CD105⁺, CD200⁺ placental stem cells are additionally CD90⁺ or CD45⁻, as detected by flow cytometry. In another specific embodiment, the isolated CD34⁻, CD10⁺, CD105⁺, CD200⁺ placental stem cells are additionally CD90⁺ and CD45⁻, as detected by flow cytometry, i.e., the cells are CD34⁻, CD10⁺, CD45⁻, CD90⁺, CD105⁺ and CD200⁺. In another specific embodiment, said CD34⁻, CD10⁺, CD45⁻, CD90⁺, CD105⁺, CD200⁺ cells are additionally CD80⁻ and CD86⁻.

In certain embodiments, said placental stem cells are CD34⁻, CD10⁺, CD105⁺ and CD200⁺, and one or more of CD38⁻, CD45⁻, CD80⁻, CD86⁻, CD133⁻, HLA-DR,DP,DQ⁻, SSEA3⁻, SSEA4⁻, CD29⁺, CD44⁺, CD73⁺, CD90⁺, CD105⁺, HLA-A,B,C⁺, PDL1⁺, ABC-p⁺, and/or OCT-4⁺, as detected by flow cytometry. In other embodiments, any of the CD34⁻, CD10⁺, CD105⁺ cells described above are additionally one or more of CD29⁺, CD38⁻, CD44⁺, CD54⁺, SH3⁺ or SH4⁺. In another specific embodiment, the cells are additionally CD44⁺. In another specific embodiment of any of the isolated CD34⁻, CD10⁺, CD105⁺ placental stem cells above, the cells are additionally one or more of CD117⁻, CD133⁻, KDR⁻ (VEGFR2⁻), HLA-A,B,C⁺, HLA-DP,DQ,DR⁻, or Programmed Death-1 Ligand (PDL1)⁺, or any combination thereof.

In another embodiment, the CD34⁻, CD10⁺, CD105⁺ cells are additionally one or more of CD13⁺, CD29⁺, CD33⁺, CD38⁻, CD44⁺, CD45⁻, CD54⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, or Programmed Death-1 Ligand (PDL1)⁺, or any combination thereof. In another embodiment, the CD34⁻, CD10⁺, CD105⁺ cells are additionally CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, and Programmed Death-1 Ligand (PDL1)⁺.

In another specific embodiment, any of the placental stem cells described herein are additionally ABC-p⁺, as detected by flow cytometry, or OCT-4⁺ (POU5F1⁺), as determined by reverse-transcriptase polymerase chain reaction (RT-PCR), wherein ABC-p is a placenta-specific ABC transporter protein (also known as breast cancer resistance protein (BCRP) and as mitoxantrone resistance protein (MXR)), and OCT-4 is the Octamer-4 protein (POU5F1).

In another specific embodiment, any of the placental stem cells described herein are additionally SSEA3⁻ or SSEA4⁻, as determined by flow cytometry, wherein SSEA3 is Stage Specific Embryonic Antigen 3, and SSEA4 is Stage Specific Embryonic Antigen 4. In another specific embodiment, any of the placental stem cells described herein are additionally SSEA3⁻ and SSEA4⁻.

In another specific embodiment, any of the placental stem cells described herein are additionally one or more of MHC-I⁺ (e.g., HLA-A,B,C⁺), MHC-II⁻ (e.g., HLA-DP,DQ,DR⁻) or HLA-G⁻. In another specific embodiment, any of the placental stem cells described herein are additionally one or more of MHC-I⁺ (e.g., HLA-A,B,C⁺), MHC-II⁻ (e.g., HLA-DP,DQ,DR⁻) and HLA-G⁻.

Also provided herein are populations of the isolated placental stem cells, or populations of cells, e.g., populations of placental cells, comprising, e.g., that are enriched for, the isolated placental stem cells, that are useful in the compositions and methods disclosed herein. Preferred populations of cells comprising the isolated placental stem cells, wherein the populations of cells comprise, e.g., at least 10%, 15%, 20%, 25%, 30%, 35%, 40%, 45%, 50%, 55%, 60%, 65%, 70%, 75%, 80%, 85%, 90%, 95% or 98% isolated CD10⁺, CD105⁺ and CD34⁻ placental stem cells; that is, at least 10%, 15%, 20%, 25%, 30%, 35%, 40%, 45%, 50%, 55%, 60%, 65%, 70%, 75%, 80%, 85%, 90%, 95% or 98% of cells in said population are isolated CD10⁺, CD105⁺ and CD34⁻ placental stem cells. In a specific embodiment, the isolated CD34⁻, CD10⁺, CD105⁺ placental stem cells are additionally CD200⁺. In another specific embodiment, the isolated CD34⁻, CD10⁺, CD105⁺, CD200⁺ placental stem cells are additionally CD90⁺ or CD45⁻, as detected by flow cytometry. In another specific embodiment, the isolated CD34⁻, CD10⁺, CD105⁺, CD200⁺ placental stem cells are additionally CD90⁺ and CD45⁻, as detected by flow cytometry. In another specific embodiment, any of the isolated CD34⁻, CD10⁺, CD105⁺ placental stem cells described above are additionally one or more of CD29⁺, CD38⁻, CD44⁺, CD54⁺, SH3⁺ or SH4⁺. In another specific embodiment, the isolated CD34⁻, CD10⁺, CD105⁺ placental stem cells, or isolated CD34⁻, CD10⁺, CD105⁺, CD200⁺ placental stem cells, are additionally CD44⁺. In a specific embodiment of any of the populations of cells comprising isolated CD34⁻, CD10⁺, CD105⁺ placental stem cells above, the isolated placental stem cells are additionally one or more of CD13⁺, CD29⁺, CD33⁺, CD38⁻, CD44⁺, CD45⁻, CD54⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, or Programmed Death-1 Ligand (PDL1)⁺, or any combination thereof. In another specific embodiment, the CD34⁻, CD10⁺, CD105⁺ cells are additionally CD13⁺, CD29⁺, CD33⁺, CD38⁻, CD44⁺, CD45⁻, CD54/ICAM⁺, CD62E⁻, CD62L⁻, CD62P⁻, SH3⁺ (CD73⁺), SH4⁺ (CD73⁺), CD80⁻, CD86⁻, CD90⁺, SH2⁺ (CD105⁺), CD106/VCAM⁺, CD117⁻, CD144/VE-cadherin^{low}, CD184/CXCR4⁻, CD200⁺, CD133⁻, OCT-4⁺, SSEA3⁻, SSEA4⁻, ABC-p⁺, KDR⁻ (VEGFR2⁻), HLA-A,B,C⁺, HLA-DP,DQ,DR⁻, HLA-G⁻, and Programmed Death-1 Ligand (PDL1)⁺.

In certain embodiments, the isolated placental stem cells useful in the compositions and methods described herein are one or more, or all, of CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4⁻, OCT-4⁺, and ABC-p⁺, wherein said isolated placental stem cells are obtained by physical and/or enzymatic disruption of placental tissue. In a specific embodiment, the isolated placental stem cells are OCT-4⁺ and ABC-p⁺. In another specific embodiment, the isolated placental stem cells are OCT-4⁺ and CD34⁻, wherein said isolated placental stem cells have at least one of the following characteristics: CD10⁺, CD29⁺, CD44⁺, CD45⁻, CD54⁺, CD90⁺, SH3⁺, SH4⁺, SSEA3⁻, and SSEA4⁻. In another specific embodiment, the isolated placental stem cells are OCT-4⁺, CD34⁻, CD10⁺, CD29⁺, CD44⁺, CD45⁻, CD54⁺, CD90⁺, SH3⁺, SH4⁺, SSEA3⁻, and SSEA4⁻. In another embodiment, the isolated placental stem cells are OCT-4⁺, CD34⁻, SSEA3⁻, and SSEA4⁻. In another specific embodiment, the isolated placental stem cells are OCT-4⁺ and CD34⁻, and is either SH2⁺ or SH3⁺. In another specific embodiment, the isolated placental stem cells are OCT-4⁺, CD34⁻, SH2⁺, and SH3⁺. In another specific embodiment, the isolated placental stem cells are OCT-4⁺, CD34⁻, SSEA3⁻, and SSEA4⁻, and are either SH2⁺ or SH3⁺. In another specific embodiment, the isolated placental stem cells are OCT-4⁺ and CD34⁻, and either SH2⁺ or SH3⁺, and is at least one of CD10⁺, CD29⁺, CD44⁺, CD45⁻, CD54⁺, CD90⁺, SSEA3⁻, or SSEA4⁻. In another specific embodiment, the isolated placental stem cells are OCT-4⁺, CD34⁻, CD10⁺, CD29⁺, CD44⁺, CD45⁻, CD54⁺, CD90⁺, SSEA3⁻, and SSEA4⁻, and either SH2⁺ or SH3⁺.

In another embodiment, the isolated placental stem cells useful in the compositions and methods disclosed herein are SH2⁺, SH3⁺, SH4⁺ and OCT-4⁺. In another specific embodiment, the isolated placental stem cells are CD10⁺, CD29⁺, CD44⁺, CD54⁺, CD90⁺, CD34⁻, CD45⁻, SSEA3⁻, or SSEA4⁻. In another embodiment, the isolated placental stem cells are SH2⁺, SH3⁺, SH4⁺, SSEA3⁻ and SSEA4⁻. In another specific embodiment, the isolated placental stem cells are SH2⁺, SH3⁺, SH4⁺, SSEA3⁻ and SSEA4⁻, CD10⁺, CD29⁺, CD44⁺, CD54⁺, CD90⁺, OCT-4⁺, CD34⁻ or CD45⁻.

In another embodiment, the isolated placental stem cells useful in the compositions and methods disclosed herein are CD10⁺, CD29⁺, CD34⁻, CD44⁺, CD45⁻, CD54⁺, CD90⁺, SH2⁺, SH3⁺, and SH4⁺; wherein said isolated placental stem cells are additionally one or more of OCT-4⁺, SSEA3⁻ or SSEA4⁻.

In certain embodiments, isolated placental stem cells useful in the compositions and methods disclosed herein are CD200⁺ or HLA-G⁻. In a specific embodiment, the isolated placental stem cells are CD200⁺ and HLA-G⁻. In another specific embodiment, the isolated placental stem cells are additionally CD73⁺ and CD105⁺. In another specific embodiment, the isolated placental stem cells are additionally CD34⁻, CD38⁻ or CD45⁻. In another specific embodiment, the isolated placental stem cells are additionally CD34⁻, CD38⁻ and CD45⁻. In another specific embodiment, said placental stem cells are CD34⁻, CD38⁻, CD45⁻, CD73⁺ and CD105⁺. In another specific embodiment, said isolated CD200⁺ or HLA-G⁻ placental stem cells facilitate the formation of embryoid-like bodies in a population of placental cells comprising the isolated placental cells, under conditions that allow the formation of embryoid-like bodies. In another specific embodiment, the isolated placental stem cells are isolated away from placental cells that are not stem or multipotent cells. In another specific embodiment, said isolated placental stem cells are isolated away from placental cells that do not display this combination of markers.

In another embodiment, a cell population useful in the compositions and methods described herein is a population of cells comprising, e.g., that is enriched for, CD200⁺, HLA-G⁻ stem cells. In a specific embodiment, said population is a population of placental stem cells. In various embodiments, at least about 10%, at least about 20%, at least about 30%, at least about 40%, at least about 50%, or at least about 60% of cells in said cell population are isolated CD200⁺, HLA-G⁻ placental stem cells. Preferably, at least about 70% of cells in said cell population are isolated CD200⁺, HLA-G⁻ placental stem cells. More preferably, at least about 90%, 95%, or 99% of said cells are isolated CD200⁺, HLA-G⁻ placental stem cells. In a specific embodiment of the cell populations, said isolated CD200⁺, HLA-G⁻ placental stem cells are also CD73⁺ and CD105⁺. In another specific embodiment, said isolated CD200⁺, HLA-G⁻ placental stem cells are also CD34⁻, CD38⁻ or CD45⁻. In another specific embodiment, said isolated CD200⁺, HLA-G⁻ placental stem cells are also CD34⁻, CD38⁻, CD45⁻, CD73⁺ and CD105⁺. In another embodiment, said cell population produces one or more embryoid-like bodies when cultured under conditions that allow the formation of embryoid-like bodies. In another specific embodiment, said cell population is isolated away from placental cells that are not stem cells. In another specific embodiment, said isolated CD200⁺, HLA-G⁻ placental stem cells are isolated away from placental cells that do not display these markers.

In another embodiment, the isolated placental stem cells useful in the compositions and methods described herein are CD73⁺, CD105⁺, and CD200⁺. In another specific embodiment, the isolated placental stem cells are HLA-G⁻. In another specific embodiment, the isolated placental stem cells are CD34⁻, CD38⁻ or CD45⁻. In another specific embodiment, the isolated placental stem cells are CD34⁻, CD38⁻ and CD45⁻. In another specific embodiment, the isolated placental stem cells are CD34⁻, CD38⁻, CD45⁻, and HLA-G⁻. In another specific embodiment, the isolated CD73⁺, CD105⁺, and CD200⁺ placental stem cells facilitate the formation of one or more embryoid-like bodies in a population of placental cells comprising the isolated placental stem cells, when the population is cultured under conditions that allow the formation of embryoid-like bodies. In another specific embodiment, the isolated placental stem cells are isolated away from placental cells that are not the isolated placental stem cells. In another specific embodiment, the isolated placental stem cells are isolated away from placental cells that do not display these markers.

In another embodiment, a cell population useful in the compositions and methods described herein is a population of cells comprising, e.g., that is enriched for, isolated CD73⁺, CD105⁺, CD200⁺ placental stem cells. In various embodiments, at least about 10%, at least about 20%, at least about 30%, at least about 40%, at least about 50%, or at least about 60% of cells in said cell population are isolated CD73⁺, CD105⁺, CD200⁺ placental stem cells. In another embodiment, at least about 70% of said cells in said population of cells are isolated CD73⁺, CD105⁺, CD200⁺ placental stem cells. In another embodiment, at least about 90%, 95% or 99% of cells in said population of cells are isolated CD73⁺, CD105⁺, CD200⁺ placental stem cells. In a specific embodiment of said populations, the isolated placental stem cells are HLA-G⁻. In another specific embodiment, the isolated placental stem cells are additionally CD34⁻, CD38⁻ or CD45⁻. In another specific embodiment, the isolated placental stem cells are additionally CD34⁻, CD38⁻ and CD45⁻. In another specific embodiment, the isolated placental stem cells are additionally CD34⁻, CD38⁻, CD45⁻, and HLA-G⁻. In another specific embodiment, said population of cells produces one or more embryoid-like bodies when cultured under conditions that allow the formation of embryoid-like bodies. In another specific embodiment, said population of placental stem cells is isolated away from placental cells that are not stem cells. In another specific embodiment, said population of placental stem cells is isolated away from placental cells that do not display these characteristics.

In certain other embodiments, the isolated placental stem cells are one or more of CD10⁺, CD29⁺, CD34⁻, CD38⁻, CD44⁺, CD45⁻, CD54⁺, CD90⁺, SH2⁺, SH3⁺, SH4⁺, SSEA3−, SSEA4⁻, OCT-4⁺, HLA-G⁻ or ABC-p⁺. In a specific embodiment, the isolated placental stem cells are CD10⁺, CD29⁺, CD34⁻, CD38⁻, CD44⁺, CD45⁻, CD54⁺, CD90⁺, SH2⁺, SH3⁺, SH4⁺, SSEA3−, SSEA4⁻, and OCT-4⁺. In another specific embodiment, the isolated placental stem cells are CD10⁺, CD29⁺, CD34⁻, CD38⁻, CD45⁻, CD54⁺, SH2⁺, SH3⁺, and SH4⁺. In another specific embodiment, the isolated placental stem cells are CD10⁺, CD29⁺, CD34⁻, CD38⁻, CD45⁻, CD54⁺, SH2⁺, SH3⁺, SH4⁺ and OCT-4⁺. In another specific embodiment, the isolated placental stem cells are CD10⁺, CD29⁺, CD34⁻, CD38⁻, CD44⁺, CD45⁻, CD54⁺, CD90⁺, HLA-G⁻, SH2⁺, SH3⁺, SH4⁺. In another specific embodiment, the isolated placental stem cells are OCT-4⁺ and ABC-p⁺. In another specific embodiment, the isolated placental stem cells are SH2⁺, SH3⁺, SH4⁺ and OCT-4⁺. In another embodiment, the isolated placental stem cells are OCT-4⁺, CD34⁻, SSEA3⁻, and SSEA4⁻. In a specific embodiment, said isolated OCT-4⁺, CD34⁻, SSEA3⁻, and SSEA4⁻ placental stem cells are additionally CD10⁺, CD29⁺, CD34⁻, CD44⁺, CD45⁻, CD54⁺, CD90⁺, SH2⁺, SH3⁺, and SH4⁺. In another embodiment, the isolated placental stem cells are OCT-4⁺ and CD34⁻, and either SH3⁺ or SH4⁺. In another embodiment, the isolated placental stem cells are CD34⁻ and either CD10⁺, CD29⁺, CD44⁺, CD54⁺, CD90⁺, or OCT-4⁺.

In another embodiment, the isolated placental stem cells useful in the compositions and methods described herein are CD200⁺ and OCT-4⁺. In a specific embodiment, the isolated placental stem cells are CD73⁺ and CD105⁺. In another specific embodiment, said isolated placental stem cells are HLA-G⁻. In another specific embodiment, said isolated CD200⁺, OCT-4⁺ placental stem cells are CD34⁻, CD38⁻ or CD45⁻. In another specific embodiment, said isolated CD200⁺, OCT-4⁺ placental stem cells are CD34⁻, CD38⁻ and CD45⁻. In another specific embodiment, said isolated CD200⁺, OCT-4⁺ placental stem cells are CD34⁻, CD38⁻, CD45⁻, CD73⁺, CD105⁺ and HLA-G⁻. In another specific embodiment, the isolated CD200⁺, OCT-4⁺ placental stem cells facilitate the production of one or more embryoid-like bodies by a population of placental cells that comprises the isolated cells, when the population is cultured under conditions that allow the formation of embryoid-like bodies. In another specific embodiment, said isolated CD200⁺, OCT-4⁺ placental stem cells are isolated away from placental cells that are not stem cells. In another specific embodiment, said isolated CD200⁺, OCT-4⁺ placental stem cells are isolated away from placental cells that do not display these characteristics.

In another embodiment, a cell population useful in the compositions and methods described herein is a population of cells comprising, e.g., that is enriched for, CD200⁺, OCT-4⁺ placental stem cells. In various embodiments, at least about 10%, at least about 20%, at least about 30%, at least about 40%, at least about 50%, or at least about 60% of cells in said cell population are isolated CD200⁺, OCT-4⁺ placental stem cells. In another embodiment, at least about 70% of said cells are said isolated CD200⁺, OCT-4⁺ placental stem cells. In another embodiment, at least about 80%, 90%, 95%, or 99% of cells in said cell population are said isolated CD200⁺, OCT-4⁺ placental stem cells. In a specific embodiment of the isolated populations, said isolated CD200⁺, OCT-4⁺ placental stem cells are additionally CD73⁺ and CD105⁺. In another specific embodiment, said isolated CD200⁺, OCT-4⁺ placental stem cells are additionally HLA-G⁻. In another specific embodiment, said isolated CD200⁺, OCT-4⁺ placental stem cells are additionally CD34⁻, CD38⁻ and CD45⁻. In another specific embodiment, said isolated CD200⁺, OCT-4⁺ placental stem cells are additionally CD34⁻, CD38⁻, CD45⁻, CD73⁺, CD105⁺ and HLA-G⁻. In another specific embodiment, the cell population produces one or more embryoid-like bodies when cultured under conditions that allow the formation of embryoid-like bodies. In another specific embodiment, said cell population is isolated away from placental cells that are not isolated CD200⁺, OCT-4⁺ placental cells. In another specific embodiment, said cell population is isolated away from placental cells that do not display these markers.

In another embodiment, the isolated placental stem cells useful in the compositions and methods described herein are CD73⁺, CD105⁺ and HLA-G⁻. In another specific embodiment, the isolated CD73⁺, CD105⁺ and HLA-G⁻ placental stem cells are additionally CD34⁻, CD38⁻ or CD45⁻. In another specific embodiment, the isolated CD73⁺, CD105⁺, HLA-G⁻ placental stem cells are additionally CD34⁻, CD38⁻ and CD45⁻. In another specific embodiment, the isolated CD73⁺, CD105⁺, HLA-G⁻ placental stem cells are additionally OCT-4⁺. In another specific embodiment, the isolated CD73⁺, CD105⁺, HLA-G⁻ placental stem cells are additionally CD200⁺. In another specific embodiment, the isolated CD73⁺, CD105⁺, HLA-G⁻ placental stem cells are additionally CD34⁻, CD38⁻, CD45⁻, OCT-4⁺ and CD200⁺. In another specific embodiment, the isolated CD73⁺, CD105⁺, HLA-G⁻ placental stem cells facilitate the formation of embryoid-like bodies in a population of placental stem cells comprising said cells, when the population is cultured under conditions that allow the formation of embryoid-like bodies. In another specific embodiment, the isolated CD73⁺, CD105⁺, HLA-G⁻ placental stem cells are isolated away from placental cells that are not the isolated CD73⁺, CD105⁺, HLA-G⁻ placental stem cells. In another specific embodiment, said the isolated CD73⁺, CD105⁺, HLA-G⁻ placental stem cells are isolated away from placental cells that do not display these markers.

In another embodiment, a cell population useful in the compositions and methods described herein is a population of cells comprising, e.g., that is enriched for, isolated CD73⁺, CD105⁺ and HLA-G⁻ placental stem cells. In various embodiments, at least about 10%, at least about 20%, at least about 30%, at least about 40%, at least about 50%, or at least about 60% of cells in said population of cells are isolated CD73⁺, CD105⁺, HLA-G⁻ placental stem cells. In another embodiment, at least about 70% of cells in said population of cells are isolated CD73⁺, CD105⁺, HLA-G⁻ placental stem cells. In another embodiment, at least about 90%, 95% or 99% of cells in said population of cells are isolated CD73⁺, CD105⁺, HLA-G⁻ placental stem cells. In a specific embodiment of the above populations, said isolated CD73⁺, CD105⁺, HLA-G⁻ placental stem cells are additionally CD34⁻, CD38⁻ or CD45⁻. In another specific embodiment, said isolated CD73⁺, CD105⁺, HLA-G⁻ placental stem cells are additionally CD34⁻, CD38⁻ and CD45⁻. In another specific embodiment, said isolated CD73⁺, CD105⁺, HLA-G⁻ placental stem cells are additionally OCT-4⁺. In another specific embodiment, said isolated CD73⁺, CD105⁺, HLA-G⁻ placental stem cells are additionally CD200⁺. In another specific embodiment, said isolated CD73⁺, CD105⁺, HLA-G⁻ placental stem cells are additionally CD34⁻, CD38⁻, CD45⁻, OCT-4⁺ and CD200⁺. In another specific embodiment, said cell population is isolated away from placental cells that are not CD73⁺, CD105⁺, HLA-G⁻ placental stem cells. In another specific embodiment, said cell population is isolated away from placental cells that do not display these markers.

In another embodiment, the isolated placental stem cells useful in the compositions and methods described herein are CD73⁺ and CD105⁺ and facilitate the formation of one or more embryoid-like bodies in a population of isolated placental cells comprising said CD73⁺, CD105⁺ cells when said population is cultured under conditions that allow formation of embryoid-like bodies. In another specific embodiment, said isolated CD73⁺, CD105⁺ placental stem cells are additionally CD34⁻, CD38⁻ or CD45⁻. In another specific embodiment, said isolated CD73⁺, CD105⁺ placental stem cells are additionally CD34⁻, CD38⁻ and CD45⁻. In another specific embodiment, said isolated CD73⁺, CD105⁺ placental stem cells are additionally OCT-4⁺. In another specific embodiment, said isolated CD73⁺, CD105⁺ placental stem cells are additionally OCT-4⁺, CD34⁻, CD38⁻ and CD45⁻. In another specific embodiment, said isolated CD73⁺, CD105⁺ placental stem cells are isolated away from placental cells that are not said cells. In another specific embodiment, said isolated CD73⁺, CD105⁺ placental stem cells are isolated away from placental cells that do not display these characteristics.

In another embodiment, a cell population useful in the compositions and methods described herein is a population of cells comprising, e.g., that is enriched for, isolated placental stem cells that are CD73⁺, CD105⁺ and facilitate the formation of one or more embryoid-like bodies in a population of isolated placental cells comprising said cells when said population is cultured under conditions that allow formation of embryoid-like bodies. In various embodiments, at least about 10%, at least about 20%, at least about 30%, at least about 40%, at least about 50%, or at least about 60% of cells in said population of cells are said isolated CD73⁺, CD105⁺ placental stem cells. In another embodiment, at least about 70% of cells in said population of cells are said isolated CD73⁺, CD105⁺ placental stem cells. In another embodiment, at least about 90%, 95% or 99% of cells in said population of cells are said isolated CD73⁺, CD105⁺ placental stem cells. In a specific embodiment of the above populations, said isolated CD73⁺, CD105⁺ placental stem cells are additionally CD34⁻, CD38⁻ or CD45⁻. In another specific embodiment, said isolated CD73⁺, CD105⁺ placental stem cells are additionally CD34⁻, CD38⁻ and CD45⁻. In another specific embodiment, said isolated CD73⁺, CD105⁺ placental stem cells are additionally OCT-4⁺. In another specific embodiment, said isolated CD73⁺, CD105⁺ placental stem cells are additionally CD200⁺. In another specific embodiment, said isolated CD73⁺, CD105⁺ placental stem cells are additionally CD34⁻, CD38⁻, CD45⁻, OCT-4⁺ and CD200⁺. In another specific embodiment, said cell population is isolated away from placental cells that are not said isolated CD73⁺, CD105⁺ placental stem cells. In another specific embodiment, said cell population is isolated away from placental cells that do not display these markers.

In another embodiment, the isolated placental stem cells useful in the compositions and methods described herein are OCT-4⁺ and facilitate formation of one or more embryoid-like bodies in a population of isolated placental cells comprising said cells when cultured under conditions that allow formation of embryoid-like bodies. In a specific embodiment, said isolated OCT-4⁺ placental stem cells are additionally CD73⁺ and CD105⁺. In another specific embodiment, said isolated OCT-4⁺ placental stem cells are additionally CD34⁻, CD38⁻, or CD45⁻. In another specific embodiment, said isolated OCT-4⁺ placental stem cells are additionally CD200⁺. In another specific embodiment, said isolated OCT-4⁺ placental stem cells are additionally CD73⁺, CD105⁺, CD200⁺, CD34⁻, CD38⁻, and CD45⁻. In another specific embodiment, said isolated OCT-4⁺ placental stem cells are isolated away from placental cells that are not OCT-4⁺ placental stem cells. In another specific embodiment, said isolated OCT-4⁺ placental stem cells are isolated away from placental cells that do not display these characteristics.

In another embodiment, a cell population useful in the compositions and methods described herein is a population of cells comprising, e.g., that is enriched for, isolated placental stem cells that are OCT-4⁺ and facilitate the formation of one or more embryoid-like bodies in a population of isolated placental cells comprising said cells when said population is cultured under conditions that allow formation of embryoid-like bodies. In various embodiments, at least about 10%, at least about 20%, at least about 30%, at least about 40%, at least about 50%, or at least about 60% of cells in said population of cells are said isolated OCT-4⁺ placental stem cells. In another embodiment, at least about 70% of cells in said population of cells are said isolated OCT-4⁺ placental stem cells. In another embodiment, at least about 80%, 90%, 95% or 99% of cells in said population of cells are said isolated OCT-4⁺ placental stem cells. In a specific embodiment of the above populations, said isolated OCT-4⁺ placental stem cells are additionally CD34⁻, CD38⁻ or CD45⁻. In another specific embodiment, said isolated OCT-4⁺ placental stem cells are additionally CD34⁻, CD38⁻ and CD45⁻. In another specific embodiment, said isolated OCT-4⁺ placental stem cells are additionally CD73⁺ and CD105⁺. In another specific embodiment, said isolated OCT-4⁺ placental stem cells are additionally CD200⁺. In another specific embodiment, said isolated OCT-4⁺ placental stem cells are additionally CD73⁺, CD105⁺, CD200⁺, CD34⁻, CD38⁻, and CD45⁻. In another specific embodiment, said cell population is isolated away from placental cells that are not said cells. In another specific embodiment, said cell population is isolated away from placental cells that do not display these markers.

In another embodiment, the isolated placental stem cells useful in the compositions and methods described herein are isolated HLA-A,B,C⁺, CD45⁻, CD133⁻ and CD34⁻ placental stem cells. In another embodiment, a cell population useful in the compositions and methods described herein is a population of cells comprising isolated placental stem cells, wherein at least about 70%, at least about 80%, at least about 90%, at least about 95% or at least about 99% of cells in said population of cells are isolated HLA-A,B,C⁺, CD45⁻, CD133⁻ and CD34⁻ placental stem cells. In a specific embodiment, said isolated placental stem cell or population of isolated placental stem cells is isolated away from placental cells that are not HLA-A,B,C⁺, CD45⁻, CD133⁻ and CD34⁻ placental stem cells. In another specific embodiment, said isolated placental stem cells are non-maternal in origin. In another specific embodiment, said population of isolated placental stem cells are substantially free of maternal components; e.g., at least about 40%, 45%, 5-0%, 55%, 60%, 65%, 70%, 75%, 90%, 85%, 90%, 95%, 98% or 99% of said cells in said population of isolated placental stem cells are non-maternal in origin.

In another embodiment, the isolated placental stem cells useful in the compositions and methods described herein are isolated CD10⁺, CD13⁺, CD33⁺, CD45⁻, CD117⁻ and CD133⁻ placental stem cells. In another embodiment, a cell population useful in the compositions and methods described herein is a population of cells comprising isolated placental stem cells, wherein at least about 70%, at least about 80%, at least about 90%, at least about 95% or at least about 99% of cells in said population of cells are isolated CD10⁺, CD13⁺, CD33⁺, CD45⁻, CD117⁻ and CD133⁻ placental stem cells. In a specific embodiment, said isolated placental stem cells or population of isolated placental stem cells is isolated away from placental cells that are not said isolated placental stem cells. In another specific embodiment, said isolated CD10⁺, CD13⁺, CD33⁺, CD45⁻, CD117⁻ and CD133⁻ placental stem cells are non-maternal in origin, i.e., have the fetal genotype. In another specific embodiment, at least about 40%, 45%, 50%, 55%, 60%, 65%, 70%, 75%, 90%, 85%, 90%, 95%, 98% or 99% of said cells in said population of isolated placental stem cells, are non-maternal in origin. In another specific embodiment, said isolated placental stem cells or population of isolated placental stem cells are isolated away from placental cells that do not display these characteristics.

In another embodiment, the isolated placental stem cells useful in the compositions and methods described herein are isolated CD10⁺ CD33⁻, CD44⁺, CD45⁻, and CD117⁻ placental stem cells. In another embodiment, a cell population useful in the compositions and methods described herein is a population of cells comprising, e.g., enriched for, isolated placental stem cells, wherein at least about 70%, at least about 80%, at least about 90%, at least about 95% or at least about 99% of cells in said population of cells are isolated CD10⁺ CD33⁻, CD44⁺, CD45⁻, and CD117⁻ placental stem cells. In a specific embodiment, said isolated placental stem cell or population of isolated placental stem cells is isolated away from placental cells that are not said cells. In another specific embodiment, said isolated placental stem cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental stem cell or population of isolated placental stem cells is isolated away from placental cells that do not display these markers.

In another embodiment, the isolated placental stem cells useful in the compositions and methods described herein are isolated CD10⁺ CD13⁻, CD33⁻, CD45⁻, and CD117⁻ placental stem cells. In another embodiment, a cell population useful in the compositions and methods described herein is a population of cells comprising, e.g., enriched for, isolated CD10⁺, CD13⁻, CD33⁻, CD45⁻, and CD117⁻ placental stem cells, wherein at least about 70%, at least about 80%, at least about 90%, at least about 95% or at least about 99% of cells in said population are CD10+ CD13⁻, CD33⁻, CD45⁻, and CD117⁻ placental stem cells. In a specific embodiment, said isolated placental stem cells or population of isolated placental stem cells are isolated away from placental cells that are not said cells. In another specific embodiment, said isolated placental stem cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental stem cells or population of isolated placental stem cells is isolated away from placental cells that do not display these characteristics.

In another embodiment, the isolated placental stem cells useful in the compositions and methods described herein are HLA A,B,C⁺, CD45⁻, CD34⁻, and CD133⁻, and are additionally CD10⁺, CD13⁺, CD38⁺, CD44⁺, CD90⁺, CD105⁺, CD200⁺ and/or HLA-G⁻, and/or negative for CD117. In another embodiment, a cell population useful in the compositions and methods described herein is a population of cells comprising isolated placental stem cells, wherein at least about 20%, 25%, 30%, 35%, 40%, 45%, 50%, 55%, 60%, 65%, 70%, 75%, 80%, 85%, 90%, 95%, 98% or about 99% of the cells in said population are isolated placental stem cells that are HLA A,B,C⁻, CD45⁻, CD34⁻, CD133⁻, and that are additionally positive for CD10, CD13, CD38, CD44, CD90, CD105, CD200, and/or negative for CD117 and/or HLA-G. In a specific embodiment, said isolated placental stem cells or population of isolated placental stem cells are isolated away from placental cells that are not said cells. In another specific embodiment, said isolated placental stem cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin. In another specific embodiment, said isolated placental stem cells or population of isolated placental stem cells are isolated away from placental cells that do not display these markers.

In another embodiment, the isolated placental stem cells useful in the compositions and methods described herein are isolated placental stem cells that are CD200⁺ and CD10⁺, as determined by antibody binding, and CD117⁻, as determined by both antibody binding and RT-PCR. In another embodiment, the isolated placental stem cells useful in the compositions and methods described herein are isolated placental stem cells that are CD10⁺, CD29⁻, CD54⁺, CD200⁺, HLA-G⁻, MHC class I⁺ and β-2-microglobulin. In another embodiment, isolated placental stem cells useful in the compositions and methods described herein are placental stem cells wherein the expression of at least one cellular marker is at least two-fold higher than for a mesenchymal stem cell (e.g., a bone marrow-derived mesenchymal stem cell). In another specific embodiment, said isolated placental stem cells are non-maternal in origin. In another specific embodiment, at least about 40%, 45%, 50%, 55%, 60%, 65%, 70%, 75%, 90%, 85%, 90%, 95%, 98% or 99% of said cells in said cell population are non-maternal in origin.

In another embodiment, the isolated placental stem cells useful in the compositions and methods described herein are isolated placental stem cells that are one or more of CD10⁺, CD29⁺, CD44⁺, CD45⁻, CD54/ICAM⁺, CD62E⁻, CD62L⁻, CD62P⁻, CD80⁻, CD86⁻, CD103⁻, CD104⁻, CD105⁺, CD106/VCAM⁺, CD144/VE-cadherin^{low}, CD184/CXCR4⁻, β2-microglobulin^{low}, MHC-I^{low}, MHC-II⁻, HLA-G^{low}, and/or PDL1^{low}. In a specific embodiment, the isolated placental stem cells are at least CD29⁺ and CD54⁺. In another specific embodiment, the isolated placental stem cells are at least CD44⁺ and CD106⁺. In another specific embodiment, the isolated placental stem cells are at least CD29⁺.

In another embodiment, a cell population useful in the compositions and methods described herein comprises isolated placental stem cells, and at least 50%, 60%, 70%, 80%, 90%, 95%, 98% or 99% of the cells in said cell population are isolated placental stem cells that are one or more of CD10⁺, CD29⁺, CD44⁺, CD45⁻, CD54/ICAM⁺, CD62-E⁻, CD62-L⁻, CD62-P⁻, CD80⁻, CD86⁻, CD103⁻, CD104⁻, CD105⁺, CD106/VCAM⁺, CD144/VE-cadherin^{dim}, CD184/CXCR4⁻, β2-microglobulin^{dim}, HLA-I^{dim}, HLA-II⁻, HLA-G^{dim}, and/or PDL1^{dim}. In another specific embodiment, at least 50%, 60%, 70%, 80%, 90%, 95%, 98% or 99% of cells in said cell population are CD10⁺, CD29⁺, CD44⁺, CD45⁻, CD54/ICAM⁺, CD62-E⁻, CD62-L⁻, CD62-P⁻, CD80⁻, CD86⁻, CD103⁻, CD104⁻, CD105⁺, CD106/VCAM⁺, CD144/VE-cadherin^{dim}, CD184/CXCR4⁻, β2-microglobulin^{dim}, MHC-I^{dim}, MHC-II⁻, HLA-G^{dim}, and PDL1^{dim}. In certain embodiments, the placental stem cells express HLA-II markers when induced by interferon gamma (IFN-γ).

In another embodiment, the isolated placental stem cells useful in the compositions and methods described herein are isolated placental stem cells that are one or more, or all, of CD10⁺, CD29⁺, CD34⁻, CD38⁻, CD44⁺, CD45⁻, CD54⁺, CD90⁺, SH2⁺, SH3⁺, SH4⁺, SSEA3⁻, SSEA4⁻, OCT-4⁺, and ABC-p⁺, where ABC-p is a placenta-specific ABC transporter protein (also known as breast cancer resistance protein (BCRP) and as mitoxantrone resistance protein (MXR)), wherein said isolated placental stem cells are obtained by perfusion of a mammalian, e.g., human, placenta that has been drained of cord blood and perfused to remove residual blood.

In another specific embodiment of any of the above characteristics, expression of the cellular marker (e.g., cluster of differentiation or immunogenic marker) is determined by flow cytometry; in another specific embodiment, expression of the marker is determined by RT-PCR.

Gene profiling confirms that isolated placental stem cells, and populations of isolated placental stem cells, are distinguishable from other cells, e.g., mesenchymal stem cells, e.g., bone marrow-derived mesenchymal stem cells. The isolated placental stem cells described herein can be distinguished from, e.g., mesenchymal stem cells on the basis of the expression of one or more genes, the expression of which is significantly higher in the isolated placental stem cells in comparison to bone marrow-derived mesenchymal stem cells. In particular, the isolated placental stem cells, useful in the methods of treatment provided herein, can be distinguished from mesenchymal stem cells on the basis of the expression of one or more genes, the expression of which is significantly higher (that is, at least twofold higher) in the isolated placental stem cells than in an equivalent number of bone marrow-derived mesenchymal stem cells, wherein the one or more genes are ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, F1110781, GATA6, GPR126, GPRC5B, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, ZC3H12A, or a combination of any of the foregoing, when the cells are grown under equivalent conditions. See, e.g., U.S. Patent Application Publication No. 2007/0275362, the disclosure of which is incorporated herein by reference in its entirety. In certain specific embodiments, said expression of said one or more genes is determined, e.g., by RT-PCR or microarray analysis, e.g, using a U133-A microarray (Affymetrix). In another specific embodiment, said isolated placental stem cells express said one or more genes when cultured for a number of population doublings, e.g., anywhere from about 3 to about 35 population doublings, in a medium comprising DMEM-LG (e.g., from Gibco); 2% fetal calf serum (e.g., from Hyclone Labs.); 1× insulin-transferrin-selenium (ITS); 1× linoleic acid-bovine serum albumin (LA-BSA); 10⁻⁹ M dexamethasone (e.g., from Sigma); 10⁻⁴ M ascorbic acid 2-phosphate (e.g., from Sigma); epidermal growth factor 10 ng/mL (e.g., from R&D Systems); and platelet-derived growth factor (PDGF-BB) 10 ng/mL (e.g., from R&D Systems). In another specific embodiment, the isolated placental stem cell-specific gene is CD200.

Specific sequences for these genes can be found in GenBank at accession nos. NM_{—}001615 (ACTG2), BC065545 (ADARB1), (NM_{—}181847 (AMIGO2), AY358590 (ARTS-1), BC074884 (B4GALT6), BC008396 (BCHE), BC020196 (C11orf9), BC031103 (CD200), NM_{—}001845 (COL4A1), NM_{—}001846 (COL4A2), BC052289 (CPA4), BC094758 (DMD), AF293359 (DSC3), NM_{—}001943 (DSG2), AF338241 (ELOVL2), AY336105 (F2RL1), NM_{—}018215 (F1110781), AY416799 (GATA6), BC075798 (GPR126), NM_{—}016235 (GPRC5B), AF340038 (ICAM1), BC000844 (IER3), BC066339 (IGFBP7), BC013142 (IL1A), BT019749 (IL6), BC007461 (IL18), (BC072017) KRT18, BC075839 (KRT8), BC060825 (LIPG), BC065240 (LRAP), BC010444 (MATN2), BC011908 (MEST), BC068455 (NFE2L3), NM_{—}014840 (NUAK1), AB006755 (PCDH7), NM_{—}014476 (PDLIM3), BC126199 (PKP-2), BC090862 (RTN1), BC002538 (SERPINB9), BC023312 (ST3GAL6), BC001201 (ST6GALNAC5), BC126160 or BC065328 (SLC12A8), BC025697 (TCF21), BC096235 (TGFB2), BC005046 (VTN), and BC005001 (ZC3H12A) as of March 2008.

In certain specific embodiments, said isolated placental stem cells express each of ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, F1110781, GATA6, GPR126, GPRC5B, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, and ZC3H12A at a detectably higher level than an equivalent number of bone marrow-derived mesenchymal stem cells, when the cells are grown under equivalent conditions.

In specific embodiments, the placental stem cells express CD200 and ARTS1 (aminopeptidase regulator of type 1 tumor necrosis factor); ARTS-1 and LRAP (leukocyte-derived arginine aminopeptidase); IL6 (interleukin-6) and TGFB2 (transforming growth factor, beta 2); IL6 and KRT18 (keratin 18); IER3 (immediate early response 3), MEST (mesoderm specific transcript homolog) and TGFB2; CD200 and IER3; CD200 and IL6; CD200 and KRT18; CD200 and LRAP; CD200 and MEST; CD200 and NFE2L3 (nuclear factor (erythroid-derived 2)-like 3); or CD200 and TGFB2 at a detectably higher level than an equivalent number of bone marrow-derived mesenchymal stem cells (BM-MSCs) wherein said bone marrow-derived mesenchymal stem cells have undergone a number of passages in culture equivalent to the number of passages said isolated placental stem cells have undergone. In other specific embodiments, the placental stem cells express ARTS-1, CD200, IL6 and LRAP; ARTS-1, IL6, TGFB2, IER3, KRT18 and MEST; CD200, IER3, IL6, KRT18, LRAP, MEST, NFE2L3, and TGFB2; ARTS-1, CD200, IER3, IL6, KRT18, LRAP, MEST, NFE2L3, and TGFB2; or IER3, MEST and TGFB2 at a detectably higher level than an equivalent number of bone marrow-derived mesenchymal stem cells BM-MSCs, wherein said bone marrow-derived mesenchymal stem cells have undergone a number of passages in culture equivalent to the number of passages said isolated placental stem cells have undergone.

Expression of the above-referenced genes can be assessed by standard techniques. For example, probes based on the sequence of the gene(s) can be individually selected and constructed by conventional techniques. Expression of the genes can be assessed, e.g., on a microarray comprising probes to one or more of the genes, e.g., an Affymetrix GENECHIP® Human Genome U133A 2.0 array, or an Affymetrix GENECHIP® Human Genome U133 Plus 2.0 (Santa Clara, Calif.). Expression of these genes can be assessed even if the sequence for a particular GenBank accession number is amended because probes specific for the amended sequence can readily be generated using well-known standard techniques.

The level of expression of these genes can be used to confirm the identity of a population of isolated placental stem cells, to identify a population of cells as comprising at least a plurality of isolated placental stem cells, or the like. Populations of isolated placental stem cells, the identity of which is confirmed, can be clonal, e.g., populations of isolated placental stem cells expanded from a single isolated placental stem cell, or a mixed population of stem cells, e.g., a population of cells comprising solely isolated placental stem cells that are expanded from multiple isolated placental stem cells, or a population of cells comprising isolated placental stem cells, as described herein, and at least one other type of cell.

The level of expression of these genes can be used to select populations of isolated placental stem cells. For example, a population of cells, e.g., clonally-expanded cells, may be selected if the expression of one or more of the genes listed above is significantly higher in a sample from the population of cells than in an equivalent population of mesenchymal stem cells. Such selecting can be of a population from a plurality of isolated placental stem cell populations, from a plurality of cell populations, the identity of which is not known, etc.

Isolated placental stem cells can be selected on the basis of the level of expression of one or more such genes as compared to the level of expression in said one or more genes in, e.g., a mesenchymal stem cell control, for example, the level of expression in said one or more genes in an equivalent number of bone marrow-derived mesenchymal stem cells. In one embodiment, the level of expression of said one or more genes in a sample comprising an equivalent number of mesenchymal stem cells is used as a control. In another embodiment, the control, for isolated placental stem cells tested under certain conditions, is a numeric value representing the level of expression of said one or more genes in mesenchymal stem cells under said conditions.

The isolated placental stem cells described herein display the above characteristics (e.g., combinations of cell surface markers and/or gene expression profiles) in primary culture, or during proliferation in medium comprising, e.g., DMEM-LG (Gibco), 2% fetal calf serum (FCS) (Hyclone Laboratories), 1× insulin-transferrin-selenium (ITS), 1× linoleic-acid-bovine-serum-albumin (LA-BSA), 10⁻⁹M dexamethasone (Sigma), 10⁻⁴M ascorbic acid 2-phosphate (Sigma), epidermal growth factor (EGF) 10 ng/ml (R&D Systems), platelet derived-growth factor (PDGF-BB) 10 ng/ml (R&D Systems), and 100U penicillin/1000U streptomycin.

In certain embodiments of any of the placental stem cells disclosed herein, the cells are human. In certain embodiments of any of the placental stem cells disclosed herein, the cellular marker characteristics or gene expression characteristics are human markers or human genes.

In another specific embodiment of said isolated placental stem cells or populations of cells comprising the isolated placental stem cells, said cells or population have been expanded, for example, passaged at least, about, or no more than, 1, 2, 3, 4, 5, 6, 7, 8, 9, 10, 11, 12, 13, 14, 15, 16, 17, 18, 19, or 20 times, or proliferated for at least, about, or no more than, 1, 2, 3, 4, 5, 6, 7, 8, 9, 10, 12, 14, 16, 18, 20, 22, 24, 26, 28, 30, 32, 34, 36, 38 or 40 population doublings. In another specific embodiment of said isolated placental stem cells or populations of cells comprising the isolated placental stem cells, said cells or population are primary isolates. In another specific embodiment of the isolated placental stem cells, or populations of cells comprising isolated placental stem cells, that are disclosed herein, said isolated placental stem cells are fetal in origin (that is, have the fetal genotype).

In certain embodiments, said isolated placental stem cells do not differentiate during culturing in growth medium, i.e., medium formulated to promote proliferation, e.g., during proliferation in growth medium. In another specific embodiment, said isolated placental stem cells do not require a feeder layer in order to proliferate. In another specific embodiment, said isolated placental stem cells do not differentiate in culture in the absence of a feeder layer, solely because of the lack of a feeder cell layer.

In another embodiment, cells useful in the compositions and methods described herein are isolated placental stem cells, wherein a plurality of said isolated placental stem cells are positive for aldehyde dehydrogenase (ALDH), as assessed by an aldehyde dehydrogenase activity assay. Such assays are known in the art (see, e.g., Bostian and Betts, Biochem. J., 173, 787, (1978)). In a specific embodiment, said ALDH assay uses ALDEFLUOR® (Aldagen, Inc., Ashland, Oreg.) as a marker of aldehyde dehydrogenase activity. In a specific embodiment, said plurality is between about 3% and about 25% of cells in said population of cells. In another embodiment, said population of isolated placental stem cells shows at least three-fold, or at least five-fold, higher ALDH activity than a population of bone marrow-derived mesenchymal stem cells having about the same number of cells and cultured under the same conditions.

In certain embodiments of any of the populations of cells comprising the isolated placental stem cells described herein, the placental stem cells in said populations of cells are substantially free of cells having a maternal genotype; e.g., at least 40%, 45%, 50%, 55%, 60%, 65%, 70%, 75%, 80%, 85%, 90%, 95%, 98% or 99% of the placental stem cells in said population have a fetal genotype.

In a specific embodiment of any of the above isolated placental stem cells or cell populations of isolated placental stem cells, the karyotype of the cells, e.g., all of the cells, or at least about 95% or about 99% of the cells in said population, is normal. In another specific embodiment of any of the above placental stem cells or cell populations, the cells, or cells in the population of cells, are non-maternal in origin.

In a specific embodiment of any of the embodiments of placental stem cells disclosed herein, the placental stem cells are genetically stable, displaying a normal diploid chromosome count and a normal karyotype.

Isolated placental stem cells, or populations of isolated placental stem cells, bearing any of the above combinations of markers, can be combined in any ratio. Any two or more of the above isolated placental stem cell populations can be combined to form an isolated placental stem cell population. For example, a population of isolated placental stem cells can comprise a first population of isolated placental stem cells defined by one of the marker combinations described above, and a second population of isolated placental stem cells defined by another of the marker combinations described above, wherein said first and second populations are combined in a ratio of about 1:99, 2:98, 3:97, 4:96, 5:95, 10:90, 20:80, 30:70, 40:60, 50:50, 60:40, 70:30, 80:20, 90:10, 95:5, 96:4, 97:3, 98:2, or about 99:1. In like fashion, any three, four, five or more of the above-described isolated placental stem cells or isolated placental stem cells populations can be combined.

Isolated placental stem cells useful in the compositions and methods described herein can be obtained, e.g., by disruption of placental tissue, with or without enzymatic digestion (see Section 4.3.7.2) or perfusion (see Section 4.3.7.3). For example, populations of isolated placental stem cells can be produced according to a method comprising perfusing a mammalian placenta that has been drained of cord blood and perfused to remove residual blood; perfusing said placenta with a perfusion solution; and collecting said perfusion solution, wherein said perfusion solution after perfusion comprises a population of placental cells that comprises isolated placental stem cells; and isolating a plurality of said isolated placental stem cells from said population of cells. In a specific embodiment, the perfusion solution is passed through both the umbilical vein and umbilical arteries and collected after it exudes from the placenta. In another specific embodiment, the perfusion solution is passed through the umbilical vein and collected from the umbilical arteries, or passed through the umbilical arteries and collected from the umbilical vein.

In various embodiments, the isolated placental stem cells, contained within a population of cells obtained from perfusion of a placenta, are at least 50%, 60%, 70%, 80%, 90%, 95%, 99% or at least 99.5% of said population of placental cells. In another specific embodiment, the isolated placental stem cells collected by perfusion comprise fetal and maternal cells. In another specific embodiment, the isolated placental stem cells collected by perfusion are at least 50%, 60%, 70%, 80%, 90%, 95%, 99% or at least 99.5% fetal cells.

In another specific embodiment, provided herein is a composition comprising a population of the isolated placental stem cells, as described herein, collected by perfusion, wherein said composition comprises at least a portion of the perfusion solution used to collect the isolated placental stem cells.

Populations of the isolated placental stem cells described herein can be produced by digesting placental tissue with a tissue-disrupting enzyme to obtain a population of placental cells comprising the cells, and isolating, or substantially isolating, a plurality of the placental stem cells from the remainder of said placental cells. The whole, or any part of, the placenta can be digested to obtain the isolated placental stem cells described herein. In specific embodiments, for example, said placental tissue can be a whole placenta (e.g., including an umbilical cord), an amniotic membrane, chorion, a combination of amnion and chorion, or a combination of any of the foregoing. In other specific embodiments, the tissue-disrupting enzyme is trypsin or collagenase. In various embodiments, the isolated placental stem cells, contained within a population of cells obtained from digesting a placenta, are at least 50%, 60%, 70%, 80%, 90%, 95%, 99% or at least 99.5% of said population of placental cells.

The populations of isolated placental stem cells described above, and populations of isolated placental stem cells generally, can comprise about, at least, or no more than, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more of the isolated placental stem cells. Populations of isolated placental stem cells useful in the methods of treatment described herein comprise at least 50%, 55%, 60%, 65%, 70%, 75%, 80%, 85%, 90%, 95%, 98%, or 99% viable isolated placental stem cells, e.g., as determined by, e.g., trypan blue exclusion

In a specific embodiment of the above-mentioned placental stem cells, the placental stem cells constitutively secrete IL-6, IL-8 and monocyte chemoattractant protein (MCP-1).

The immunosuppressive pluralities of placental stem cells described above can comprise about, at least, or no more than, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more placental stem cells.

In certain embodiments, the placental stem cells (e.g., PDACs) useful in the compositions and methods provided herein, do not express CD34, as detected by immunolocalization, after exposure to 1 to 100 ng/mL VEGF for 4 to 21 days. In a specific embodiment, said placental adherent cells are adherent to tissue culture plastic. In another specific embodiment, said population of cells induce endothelial cells to form sprouts or tube-like structures when cultured in the presence of an angiogenic factor such as vascular endothelial growth factor (VEGF), epithelial growth factor (EGF), platelet derived growth factor (PDGF) or basic fibroblast growth factor (bFGF), e.g., on a substrate such as MATRIGEL™.

In another aspect, the PDACs provided herein, a population of cells, e.g., a population of PDACs, or a population of cells wherein at least about 50%, 60%, 70%, 80%, 90%, 95% or 98% of cells in said population of cells are PDACs, secrete one or more, or all, of VEGF, HGF, IL-8, MCP-3, FGF2, follistatin, G-CSF, EGF, ENA-78, GRO, IL-6, MCP-1, PDGF-BB, TIMP-2, uPAR, or galectin-1, e.g., into culture medium in which the cell, or cells, are grown. In another embodiment, the PDACs express increased levels of CD202b, IL-8 and/or VEGF under hypoxic conditions (e.g., less than about 5% O₂) compared to normoxic conditions (e.g., about 20% or about 21% O₂).

In another embodiment, any of the PDACs or populations of cells comprising PDACs described herein can cause the formation of sprouts or tube-like structures in a population of endothelial cells in contact with said placental derived adherent cells. In a specific embodiment, the PDACs are co-cultured with human endothelial cells, which form sprouts or tube-like structures, or support the formation of endothelial cell sprouts, e.g., when cultured in the presence of extracellular matrix proteins such as collagen type I and IV, and/or angiogenic factors such as vascular endothelial growth factor (VEGF), epithelial growth factor (EGF), platelet derived growth factor (PDGF) or basic fibroblast growth factor (bFGF), e.g., in or on a substrate such as placental collagen or MATRIGEL™ for at least 4 days. In another embodiment, any of the populations of cells comprising placental derived adherent cells, described herein, secrete angiogenic factors such as vascular endothelial growth factor (VEGF), hepatocyte growth factor (HGF), platelet derived growth factor (PDGF), basic fibroblast growth factor (bFGF), or Interleukin-8 (IL-8) and thereby can induce human endothelial cells to form sprouts or tube-like structures when cultured in the presence of extracellular matrix proteins such as collagen type I and IV e.g., in or on a substrate such as placental collagen or MATRIGEL™.

In another embodiment, any of the above populations of cells comprising placental derived adherent cells (PDACs) secretes angiogenic factors. In specific embodiments, the population of cells secretes vascular endothelial growth factor (VEGF), hepatocyte growth factor (HGF), platelet derived growth factor (PDGF), basic fibroblast growth factor (bFGF), and/or interleukin-8 (IL-8). In other specific embodiments, the population of cells comprising PDACs secretes one or more angiogenic factors and thereby induces human endothelial cells to migrate in an in vitro wound healing assay. In other specific embodiments, the population of cells comprising placental derived adherent cells induces maturation, differentiation or proliferation of human endothelial cells, endothelial progenitors, myocytes or myoblasts.

4.3.3 Selecting and Producing Placental Stem Cell Populations

In certain embodiments, populations of placental stem cells can be selected, wherein the population is immunosuppressive. In one embodiment, for example, provided herein is a method of selecting a plurality of immunosuppressive placental stem cells from a plurality of placental cells, comprising selecting a population of placental cells wherein at least 10%, at least 20%, at least 30%, at least 40%, at least 50% at least 60%, at least 70%, at least 80%, at least 90%, or at least 95% of said cells are CD10⁺, CD34⁻, CD105⁺, CD200⁺ placental stem cells, and wherein said placental stem cells detectably suppresses T cell proliferation in an MLR assay. In a specific embodiment, said selecting comprises selecting stem cells that are also CD45⁻ and CD90⁺.

In another embodiment, provided herein is a method of selecting a plurality of immunosuppressive placental stem cells from a plurality of placental cells, comprising selecting a population of placental cells wherein at least 10%, at least 20%, at least 30%, at least 40%, at least 50% at least 60%, at least 70%, at least 80%, at least 90%, or at least 95% of said cells are CD200⁺, HLA-G⁻ placental stem cells, and wherein said placental stem cells detectably suppresses T cell proliferation in an MLR assay. In a specific embodiment, said selecting comprises selecting stem cells that are also CD73⁺ and CD105⁺. In another specific embodiment, said selecting comprises selecting stem cells that are also CD34⁻, CD38⁻ or CD45⁻. In another specific embodiment, said selecting comprises selecting placental stem cells that are also CD34⁻, CD38⁻, CD45⁻, CD73⁺ and CD105⁺. In another specific embodiment, said selecting also comprises selecting a plurality of placental stem cells that forms one or more embryoid-like bodies when cultured under conditions that allow the formation of embryoid-like bodies.

In another embodiment, provided herein is a method of selecting a plurality of immunosuppressive placental stem cells from a plurality of placental cells, comprising selecting a plurality of placental cells wherein at least 10%, at least 20%, at least 30%, at least 40%, at least 50% at least 60%, at least 70%, at least 80%, at least 90%, or at least 95% of said cells are CD73⁺, CD105⁺, CD200⁺ placental stem cells, and wherein said placental stem cells detectably suppresses T cell proliferation in an MLR assay. In a specific embodiment, said selecting comprises selecting stem cells that are also HLA-G⁻. In another specific embodiment, said selecting comprises selecting placental stem cells that are also CD34⁻, CD38⁻ or CD45⁻. In another specific embodiment, said selecting comprises selecting placental stem cells that are also CD34⁻, CD38⁻ and CD45⁻. In another specific embodiment, said selecting comprises selecting placental stem cells that are also CD34⁻, CD38⁻, CD45⁻, and HLA-G⁻. In another specific embodiment, said selecting additionally comprises selecting a population of placental stem cells that produces one or more embryoid-like bodies when the population is cultured under conditions that allow the formation of embryoid-like bodies.

In another embodiment, also provided herein is a method of selecting a plurality of immunosuppressive placental stem cells from a plurality of placental cells, comprising selecting a plurality of placental cells wherein at least 10%, at least 20%, at least 30%, at least 40%, at least 50% at least 60%, at least 70%, at least 80%, at least 90%, or at least 95% of said cells are CD200⁺, OCT-4⁺ placental stem cells, and wherein said placental stem cells detectably suppresses T cell proliferation in an MLR assay. In a specific embodiment, said selecting comprises selecting placental stem cells that are also CD73⁺ and CD105⁺. In another specific embodiment, said selecting comprises selecting placental stem cells that are also HLA-G⁻. In another specific embodiment, said selecting comprises selecting placental stem cells that are also CD34⁻, CD38⁻ and CD45⁻. In another specific embodiment, said selecting comprises selecting placental stem cells that are also CD34⁻, CD38⁻, CD45⁻, CD73⁺, CD105⁺ and HLA-G⁻.

In another embodiment, provided herein is a method of selecting a plurality of immunosuppressive placental stem cells from a plurality of placental cells, comprising selecting a plurality of placental cells wherein at least 10%, at least 20%, at least 30%, at least 40%, at least 50% at least 60%, at least 70%, at least 80%, at least 90%, or at least 95% of said cells are CD73⁺, CD105⁺ and HLA-G⁻ placental stem cells, and wherein said placental stem cells detectably suppresses T cell proliferation in an MLR assay. In a specific embodiment, said selecting comprises selecting placental stem cells that are also CD34⁻, CD38⁻ or CD45⁻. In another specific embodiment, said selecting comprises selecting placental stem cells that are also CD34⁻, CD38⁻ and CD45⁻. In another specific embodiment, said selecting comprises selecting placental stem cells that are also CD200⁺. In another specific embodiment, said selecting comprises selecting placental stem cells that are also CD34⁻, CD38⁻, CD45⁻, OCT-4⁺ and CD200⁺.

In another embodiment, also provided herein is provides a method of selecting a plurality of immunosuppressive placental stem cells from a plurality of placental cells, comprising selecting a plurality of placental stem cells wherein at least 10%, at least 20%, at least 30%, at least 40%, at least 50% at least 60%, at least 70%, at least 80%, at least 90%, or at least 95% of said cells are CD73⁺, CD105⁺ placental stem cells, and wherein said plurality forms one or more embryoid-like bodies under conditions that allow formation of embryoid-like bodies. In a specific embodiment, said selecting comprises selecting placental stem cells that are also CD34⁻, CD38⁻ or CD45⁻. In another specific embodiment, said selecting comprises selecting placental stem cells that are also CD34⁻, CD38⁻ and CD45⁻. In another specific embodiment, said selecting comprises selecting placental stem cells that are also OCT-4⁺. In a more specific embodiment, said selecting comprises selecting placental stem cells that are also OCT-4⁺, CD34⁻, CD38⁻ and CD45⁻.

In another embodiment, provided herein is a method of selecting a plurality of immunosuppressive placental stem cells from a plurality of placental cells, comprising selecting a plurality of placental stem cells wherein at least 10%, at least 20%, at least 30%, at least 40%, at least 50% at least 60%, at least 70%, at least 80%, at least 90%, or at least 95% of said isolated placental stem cells are OCT4⁺ stem cells, and wherein said plurality forms one or more embryoid-like bodies under conditions that allow formation of embryoid-like bodies. In a specific embodiment, said selecting comprises selecting placental stem cells that are also CD73⁺ and CD105⁺. In another specific embodiment, said selecting comprises selecting placental stem cells that are also CD34⁻, CD38⁻, or CD45⁻. In another specific embodiment, said selecting comprises selecting placental stem cells that are also CD200⁺. In a more specific embodiment, said selecting comprises selecting placental stem cells that are also CD73⁺, CD105⁺, CD200⁺, CD34⁻, CD38⁻, and CD45⁻.

Immunosuppressive populations, or pluralities, of placental stem cells can be produced according to the methods provided herein. For example, provided herein is method of producing a cell population, comprising selecting any of the pluralities of placental stem cells described above, and isolating the plurality of placental stem cells from other cells, e.g., other placental cells. In a specific embodiment, provided herein is a method of producing a cell population comprising selecting placental stem cells, wherein said placental stem cells (a) adhere to a substrate, (b) express CD200 and do not express HLA-G, or express CD73, CD105, and CD200, or express CD200 and OCT-4, or express CD73, CD105, and do not express HLA-G, or express CD73 and CD105 and facilitate the formation of one or more embryoid-like bodies in a population of placental cells that comprise the stem cell, when said population is cultured under conditions that allow formation of embryoid-like bodies, or express OCT-4 and facilitate the formation of one or more embryoid-like bodies in a population of placental cells that comprise the stem cell, when said population is cultured under conditions that allow formation of embryoid-like bodies; and (c) detectably suppress CD4⁺ or CD8⁺ T cell proliferation in an MLR or regression assay; and isolating said placental stem cells from other cells to form a cell population.

In a more specific embodiment, immunosuppressive placental stem cell populations can be produced by a method comprising selecting placental stem cells that (a) adhere to a substrate, (b) express CD200 and do not express HLA-G, and (c) detectably suppress CD4⁺ or CD8⁺ T cell proliferation in an MLR assay; and isolating said placental stem cells from other cells to form a cell population. In another specific embodiment, the method comprises selecting placental stem cells that (a) adhere to a substrate, (b) express CD73, CD105, and CD200, and (c) detectably suppress CD4⁺ or CD8⁺ T cell proliferation in an MLR; and isolating said placental stem cells from other cells to form a cell population. In another specific embodiment, provided herein is a method of producing a cell population comprising selecting placental stem cells that (a) adhere to a substrate, (b) express CD200 and OCT-4, and (c) detectably suppress CD4⁺ or CD8⁺ T cell proliferation in an MLR; and isolating said placental stem cells from other cells to form a cell population. In another specific embodiment, provided herein is a method of producing a cell population comprising selecting placental stem cells that (a) adhere to a substrate, (b) express CD73 and CD105, (c) form embryoid-like bodies when cultured under conditions allowing the formation of embryoid-like bodies, and (d) detectably suppress CD4⁺ or CD8⁺ T cell proliferation in an MLR; and isolating said placental stem cells from other cells to form a cell population. In another specific embodiment, the method comprises selecting placental stem cells that (a) adhere to a substrate, (b) express CD73 and CD105, and do not express HLA-G, and (c) detectably suppress CD4⁺ or CD8⁺ T cell proliferation in an MLR; and isolating said placental stem cells from other cells to form a cell population. A method of producing a cell population comprising selecting placental stem cells that (a) adhere to a substrate, (b) express OCT-4, (c) form embryoid-like bodies when cultured under conditions allowing the formation of embryoid-like bodies, and (d) detectably suppress CD4⁺ or CD8⁺ T cell proliferation in an MLR; and isolating said placental stem cells from other cells to form a cell population.

In a specific embodiment of the methods of producing an immunosuppressive placental stem cell population, said T cells and said placental stem cells are present in said MLR at a ratio of about 5:1. The placental stem cells used in the method can be derived from the whole placenta, or primarily from amnion, or amnion and chorion. In another specific embodiment, the placental stem cells suppress CD4⁺ or CD8⁺ T cell proliferation by at least 50%, at least 75%, at least 90%, or at least 95% in said MLR compared to an amount of T cell proliferation in said MLR in the absence of said placental stem cells. The method can additionally comprise the selection and/or production of a placental stem cell population capable of immunomodulation, e.g., suppression of the activity of, other immune cells, e.g., an activity of a natural killer (NK) cell.

4.3.4 Growth in Culture

The growth of the placental stem cells (PDACs) described herein, as for any mammalian cell, depends in part upon the particular medium selected for growth. Under optimum conditions, placental stem cells typically double in number in 3-5 days. During culture, the placental stem cells provided herein adhere to a substrate in culture, e.g. the surface of a tissue culture container (e.g., tissue culture dish plastic, fibronectin-coated plastic, and the like) and form a monolayer.

Populations of isolated placental cells that comprise the placental cells provided herein, when cultured under appropriate conditions, form embryoid-like bodies, that is, three-dimensional clusters of cells grow atop the adherent stem cell layer. Cells within the embryoid-like bodies express markers associated with very early stem cells, e.g., OCT-4, Nanog, SSEA3 and SSEA4. Cells within the embryoid-like bodies are typically not adherent to the culture substrate, as are the placental cells described herein, but remain attached to the adherent cells during culture. Embryoid-like body cells are dependent upon the adherent placental cells for viability, as embryoid-like bodies do not form in the absence of the adherent stem cells. The adherent placental cells thus facilitate the growth of one or more embryoid-like bodies in a population of placental cells that comprise the adherent placental cells. Without wishing to be bound by theory, the cells of the embryoid-like bodies are thought to grow on the adherent placental cells much as embryonic stem cells grow on a feeder layer of cells. Mesenchymal stem cells, e.g., bone marrow-derived mesenchymal stem cells, do not develop embryoid-like bodies in culture.

4.3.5 Differentiation

In certain embodiments, the placental stem cells, useful in the compositions and methods provided herein, are differentiable into different committed cell lineages. For example, in certain embodiments, the placental stem cells can be differentiated into cells of an adipogenic, chondrogenic, neurogenic, or osteogenic lineage. Such differentiation can be accomplished, e.g., by any method known in the art for differentiating, e.g., bone marrow-derived mesenchymal stem cells into similar cell lineages, or by methods described elsewhere herein. Specific methods of differentiating placental stem cells into particular cell lineages are disclosed in, e.g., U.S. Pat. No. 7,311,905, and in U.S. Patent Application Publication No. 2007/0275362, the disclosures of which are hereby incorporated by reference in their entireties.

The placental stem cells provided herein can exhibit the capacity to differentiate into a particular cell lineage in vitro, in vivo, or in vitro and in vivo. In a specific embodiment, the placental stem cells provided herein can be differentiated in vitro when placed in conditions that cause or promote differentiation into a particular cell lineage, but do not detectably differentiate in vivo, e.g., in a NOD-SCID mouse model.

4.3.6 Stem Cell Collection Composition

Placental stem cells can be collected and isolated according to the methods provided herein. Generally, stem cells are obtained from a mammalian placenta using a physiologically-acceptable solution, e.g., a stem cell collection composition. A stem cell collection composition is described in detail in related U.S. Application Publication No. 2007/0190042, entitled “Improved Composition for Collecting and Preserving Placental cells and Methods of Using the Composition” filed on Dec. 29, 2005.

The stem cell collection composition can comprise any physiologically-acceptable solution suitable for the collection and/or culture of stem cells, for example, a saline solution (e.g., phosphate-buffered saline, Kreb's solution, modified Kreb's solution, Eagle's solution, 0.9% NaCl. etc.), a culture medium (e.g., DMEM, HDMEM, etc.), and the like.

The stem cell collection composition can comprise one or more components that tend to preserve placental stem cells, that is, prevent the placental stem cells from dying, or delay the death of the placental stem cells, reduce the number of placental stem cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The stem cell collection composition can comprise one or more tissue-degrading enzymes, e.g., a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE, hyaluronidase, and the like.

The stem cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

The stem cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/1 to about 100,000 units/1); or an amiloride containing compound (e.g., amiloride, ethyl isopropyl amiloride, hexamethylene amiloride, dimethyl amiloride or isobutyl amiloride present at about 1.0 μM to about 5 μM).

4.3.7 Methods of Obtaining Placental Stem Cells

4.3.7.1 Collection and Handling of Placenta

Generally, a human placenta is recovered shortly after its expulsion after birth. In a preferred embodiment, the placenta is recovered from a patient after informed consent and after a complete medical history of the patient is taken and is associated with the placenta. Preferably, the medical history continues after delivery. Such a medical history can be used to coordinate subsequent use of the placenta or the stem cells harvested therefrom. For example, human placental stem cells can be used, in light of the medical history, for personalized medicine for the infant associated with the placenta, or for parents, siblings or other relatives of the infant.

Prior to recovery of placental stem cells, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank Inc., Cedar Knolls, N.J., ViaCord, Cord Blood Registry and Cryocell. Preferably, the placenta is gravity drained without further manipulation so as to minimize tissue disruption during cord blood recovery.

Typically, a placenta is transported from the delivery or birthing room to another location, e.g., a laboratory, for recovery of cord blood and collection of stem cells by, e.g., perfusion or tissue dissociation. The placenta is preferably transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in pending U.S. patent application Ser. No. 11/230,760, filed Sep. 19, 2005. Preferably, the placenta is delivered to the laboratory four to twenty-four hours following delivery. In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta.

The placenta, prior to stem cell collection, can be stored under sterile conditions and at either room temperature or at a temperature of 5 to 25° C. (centigrade). The placenta may be stored for a period of longer than forty eight hours, and preferably for a period of four to twenty-four hours prior to perfusing the placenta to remove any residual cord blood. The placenta is preferably stored in an anticoagulant solution at a temperature of 5 to 25° C. (centigrade). Suitable anticoagulant solutions are well known in the art. For example, a solution of heparin or warfarin sodium can be used. In a preferred embodiment, the anticoagulant solution comprises a solution of heparin (e.g., 1% w/w in 1:1000 solution). The exsanguinated placenta is preferably stored for no more than 36 hours before placental cells are collected.

The mammalian placenta or a part thereof, once collected and prepared generally as above, can be treated in any art-known manner, e.g., can be perfused or disrupted, e.g., digested with one or more tissue-disrupting enzymes, to obtain stem cells.

4.3.7.2 Physical Disruption and Enzymatic Digestion of Placental Tissue

In one embodiment, stem cells are collected from a mammalian placenta by physical disruption, e.g., enzymatic digestion, of the organ, e.g., using the stem cell collection composition described in Section 5.3.1, above. For example, the placenta, or a portion thereof, may be, e.g., crushed, sheared, minced, diced, chopped, macerated or the like, while in contact with, e.g., a buffer, medium or a stem cell collection composition, and the tissue subsequently digested with one or more enzymes. The placenta, or a portion thereof, may also be physically disrupted and digested with one or more enzymes, and the resulting material then immersed in, or mixed into, a buffer, medium or a stem cell collection composition. Any method of physical disruption can be used, provided that the method of disruption leaves a plurality, more preferably a majority, and more preferably at least 60%, 70%, 80%, 90%, 95%, 98%, or 99% of the cells in said organ viable, as determined by, e.g., trypan blue exclusion.

The placenta can be dissected into components prior to physical disruption and/or enzymatic digestion and stem cell recovery. For example, placental stem cells can be obtained from the amniotic membrane, chorion, placental cotyledons, or any combination thereof, or umbilical cord, or any combination thereof. Preferably, placental stem cells are obtained from placental tissue comprising amnion and chorion, or amnion-chorion and umbilical cord. In one embodiment, stem cells are obtained from amnion-chorion and umbilical cord in about a 1:1 weight ratio. Typically, placental stem cells can be obtained by disruption of a small block of placental tissue, e.g., a block of placental tissue that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 cubic millimeters in volume.

A preferred stem cell collection composition comprises one or more tissue-disruptive enzyme(s). Enzymatic digestion preferably uses a combination of enzymes, e.g., trypsin, chymotrypsin, elastase, collagenase, dispase, or the like. Serine proteases may be inhibited by alpha 2 microglobulin in serum and therefore the medium used for digestion is usually serum-free. EDTA and DNase are commonly used in enzyme digestion procedures to increase the efficiency of cell recovery. The digestate is preferably diluted so as to avoid trapping stem cells within the viscous digest.

Any combination of tissue digestion enzymes can be used. Typical concentrations for tissue digestion enzymes include, e.g., 50-200 U/mL for collagenase I and collagenase IV, 1-10 U/mL for dispase, and 10-100 U/mL for elastase. Proteases can be used in combination, that is, two or more proteases in the same digestion reaction, or can be used sequentially in order to liberate placental stem cells. For example, in one embodiment, a placenta, or part thereof, is digested first with an appropriate amount of collagenase I at 2 mg/ml for 30 minutes, followed by digestion with trypsin, 0.25%, for 10 minutes, at 37° C. Serine proteases are preferably used consecutively following use of other enzymes.

In another embodiment, the tissue can further be disrupted by the addition of a chelator, e.g., ethylene glycol bis(2-aminoethyl ether)-N,N,N′N′-tetraacetic acid (EGTA) or ethylenediaminetetraacetic acid (EDTA) to the stem cell collection composition comprising the stem cells, or to a solution in which the tissue is disrupted and/or digested prior to isolation of the stem cells with the stem cell collection composition.

It will be appreciated that where an entire placenta, or portion of a placenta comprising both fetal and maternal cells (for example, where the portion of the placenta comprises the chorion or cotyledons), the placental cells collected will comprise a mix of placental cells derived from both fetal and maternal sources. Where a portion of the placenta that comprises no, or a negligible number of, maternal cells (for example, amnion), the placental cells collected will comprise almost exclusively fetal placental cells.

4.3.7.3 Placental Perfusion

Placental stem cells (PDACs) can also be obtained by perfusion of the mammalian placenta. Methods of perfusing mammalian placenta to obtain stem cells are disclosed, e.g., in Hariri, U.S. Application Publication No. 2002/0123141, and in related U.S. Application Publication No. 2007/0190042, entitled “Improved Composition for Collecting and Preserving Placental cells and Methods of Using the Composition” filed on Dec. 29, 2005.

Placental stem cells can be collected by perfusion, e.g., through the placental vasculature, using, e.g., a stem cell collection composition as a perfusion solution. In one embodiment, a mammalian placenta is perfused by passage of perfusion solution through either or both of the umbilical artery and umbilical vein. The flow of perfusion solution through the placenta may be accomplished using, e.g., gravity flow into the placenta. Preferably, the perfusion solution is forced through the placenta using a pump, e.g., a peristaltic pump. The umbilical vein can be, e.g., cannulated with a cannula, e.g., a TEFLON® or plastic cannula, that is connected to a sterile connection apparatus, such as sterile tubing. The sterile connection apparatus is connected to a perfusion manifold.

In preparation for perfusion, the placenta is preferably oriented (e.g., suspended) in such a manner that the umbilical artery and umbilical vein are located at the highest point of the placenta. The placenta can be perfused by passage of a perfusion fluid, e.g., the stem cell collection composition provided herein, through the placental vasculature, or through the placental vasculature and surrounding tissue. In one embodiment, the umbilical artery and the umbilical vein are connected simultaneously to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. In another embodiment, the perfusion solution is passed through the umbilical veins and collected from the umbilical artery, or is passed through the umbilical artery and collected from the umbilical veins.

The first collection of perfusion fluid from a mammalian placenta during the exsanguination process is generally colored with residual red blood cells of the cord blood and/or placental blood. The perfusion fluid becomes more colorless as perfusion proceeds and the residual cord blood cells are washed out of the placenta. Generally from 30 to 100 ml (milliliter) of perfusion fluid is adequate to initially exsanguinate the placenta, but more or less perfusion fluid may be used depending on the observed results.

The volume of perfusion liquid used to collect placental stem cells may vary depending upon the number of stem cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with the stem cell collection composition, or a standard perfusion solution (e.g., a normal saline solution such as phosphate buffered saline (“PBS”)) with or without an anticoagulant (e.g., heparin, warfarin sodium, coumarin, bishydroxycoumarin), and/or with or without an antimicrobial agent (e.g., β-mercaptoethanol (0.1 mM); antibiotics such as streptomycin (e.g., at 40-100 μg/ml), penicillin (e.g., at 40 U/ml), amphotericin B (e.g., at 0.5 μg/ml). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In a preferred embodiment, perfusion of the placenta and collection of perfusion solution, e.g., stem cell collection composition, is repeated until the number of recovered nucleated cells falls below 100 cells/ml. The perfusates at different time points can be further processed individually to recover time-dependent populations of cells, e.g., stem cells. Perfusates from different time points can also be pooled.

Stem cells can be isolated from placenta by perfusion with a solution comprising one or more proteases or other tissue-disruptive enzymes. In a specific embodiment, a placenta or portion thereof (e.g., amniotic membrane, amnion and chorion, placental lobule or cotyledon, or combination of any of the foregoing) is brought to 25-37° C., and is incubated with one or more tissue-disruptive enzymes in 200 mL of a culture medium for 30 minutes. Cells from the perfusate are collected, brought to 4° C., and washed with a cold inhibitor mix comprising 5 mM EDTA, 2 mM dithiothreitol and 2 mM beta-mercaptoethanol. The stem cells are washed after several minutes with a cold (e.g., 4° C.) stem cell collection composition described elsewhere herein.

It will be appreciated that perfusion using the pan method, that is, whereby perfusate is collected after it has exuded from the maternal side of the placenta, results in a mix of fetal and maternal cells. As a result, the cells collected by this method comprise a mixed population of placental cells of both fetal and maternal origin. In contrast, perfusion solely through the placental vasculature, whereby perfusion fluid is passed through one or two placental vessels and is collected solely through the remaining vessel(s), results in the collection of a population of placental cells almost exclusively of fetal origin.

4.3.8 Isolation, Sorting, and Characterization of Placental Stem Cells

Stem cells from mammalian placenta, whether obtained by perfusion or enyzmatic digestion, can initially be purified from (i.e., be isolated from) other cells by Ficoll gradient centrifugation. Such centrifugation can follow any standard protocol for centrifugation speed, etc. In one embodiment, for example, cells collected from the placenta are recovered from perfusate by centrifugation at 5000×g for 15 minutes at room temperature, which separates cells from, e.g., contaminating debris and platelets. In another embodiment, placental perfusate is concentrated to about 200 ml, gently layered over Ficoll, and centrifuged at about 1100×g for 20 minutes at 22° C., and the low-density interface layer of cells is collected for further processing.

Cell pellets can be resuspended in fresh stem cell collection composition, or a medium suitable for stem cell maintenance, e.g., IMDM serum-free medium containing 2 U/ml heparin and 2 mM EDTA (GibcoBRL, NY). The total mononuclear cell fraction can be isolated, e.g., using Lymphoprep (Nycomed Pharma, Oslo, Norway) according to the manufacturer's recommended procedure.

As used herein, “isolating” placental stem cells (PDACs) means to remove at least 20%, 30%, 40%, 50%, 60%, 70%, 80%, 90%, 95% or 99% of the cells with which the stem cells are normally associated in the intact mammalian placenta. A stem cell from an organ is “isolated” when it is present in a population of cells that comprises fewer than 50% of the cells with which the stem cell is normally associated in the intact organ.

Placental stem cells obtained by perfusion or digestion can, for example, be further, or initially, isolated by differential trypsinization using, e.g., a solution of 0.05% trypsin with 0.2% EDTA (Sigma, St. Louis Mo.). Differential trypsinization is possible because placental stem cells (PDACs) typically detach from plastic surfaces within about five minutes whereas other adherent populations typically require more than 20-30 minutes incubation. The detached placental stem cells can be harvested following trypsinization and trypsin neutralization, using, e.g., Trypsin Neutralizing Solution (TNS, Cambrex). In one embodiment of isolation of adherent cells, aliquots of, for example, about 5-10×10⁶ cells are placed in each of several T-75 flasks, preferably fibronectin-coated T75 flasks. In such an embodiment, the cells can be cultured with commercially available Mesenchymal Stem Cell Growth Medium (MSCGM) (Cambrex), and placed in a tissue culture incubator (37° C., 5% CO₂). After 10 to 15 days, non-adherent cells are removed from the flasks by washing with PBS. The PBS is then replaced by MSCGM. Flasks are preferably examined daily for the presence of various adherent cell types and in particular, for identification and expansion of clusters of fibroblastoid cells.

The number and type of cells collected from a mammalian placenta can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using antibodies to CD34, one can determine, using the techniques above, whether a cell comprises a detectable amount of CD34; if so, the cell is CD34⁺. Likewise, if a cell produces enough OCT-4 RNA to be detectable by RT-PCR, or significantly more OCT-4 RNA than an adult cell, the cell is OCT-4⁺. Antibodies to cell surface markers (e.g., CD markers such as CD34) and the sequence of stem cell-specific genes, such as OCT-4, are well-known in the art.

Placental stem cells, particularly cells that have been isolated by Ficoll separation, differential adherence, or a combination of both, may be sorted using a fluorescence activated cell sorter (FACS). Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, stem cells from placenta are sorted on the basis of expression of the markers CD34, CD38, CD44, CD45, CD73, CD105, and/or OCT-4. This can be accomplished in connection with procedures to select stem cells on the basis of their adherence properties in culture. For example, an adherence selection stem can be accomplished before or after sorting on the basis of marker expression. In one embodiment, for example, cells are sorted first on the basis of their expression of CD34; CD34⁻ cells are retained, and cells that are CD200⁺, are separated from all other CD34⁻ cells. In another embodiment, cells from placenta are based on their expression of CD200. Cells that express, e.g., CD200 can, in a specific embodiment, be further sorted based on their expression of CD73 and/or CD105, or epitopes recognized by antibodies SH2, SH3 or SH4, or lack of expression of CD34, CD38 or CD45. For example, in one embodiment, placental stem cells are sorted by expression, or lack thereof, of CD200, CD73, CD105, CD34, CD38 and CD45, and placental stem cells that are CD200⁺, CD73⁺, CD105⁺, CD34⁻, CD38⁻ and CD45⁻ are isolated from other placental cells for further use.

In another embodiment, magnetic beads can be used to separate cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

Placental stem cells can also be characterized and/or sorted based on cell morphology and growth characteristics. For example, placental stem cells can be characterized as having, and/or selected on the basis of, e.g., a fibroblastoid appearance in culture. Placental stem cells can also be characterized as having, and/or be selected, on the basis of their ability to form embryoid-like bodies. In one embodiment, for example, placental stem cells that are fibroblastoid in shape, express CD73 and CD105, and produce one or more embryoid-like bodies in culture are isolated from other placental stem cells. In another embodiment, OCT-4⁺ placental stem cells that produce one or more embryoid-like bodies in culture are isolated from other placental cells.

In another embodiment, placental stem cells can be identified and characterized by a colony forming unit assay. Colony forming unit assays are commonly known in the art, such as Mesen Cult™ medium (Stem Cell Technologies, Inc., Vancouver British Columbia)

Placental stem cells can be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT cell proliferation assay (to assess proliferation). Longevity may be determined by methods well known in the art, such as by determining the maximum number of population doubling in an extended culture.

Placental stem cells can also be separated from other placental cells using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

4.3.9 Culture of Placental Stem Cells

4.3.9.1 Culture Media

Isolated placental stem cells, or placental stem cell population, or cells or placental tissue from which placental stem cells grow out, can be used to initiate, or seed, cell cultures. Cells are generally transferred to sterile tissue culture vessels either uncoated or coated with extracellular matrix or ligands such as laminin, collagen (e.g., native or denatured), gelatin, fibronectin, ornithine, vitronectin, and extracellular membrane protein (e.g., MATRIGEL (BD Discovery Labware, Bedford, Mass.)).

Placental stem cells can be cultured in any medium, and under any conditions, recognized in the art as acceptable for the culture of stem cells. In one embodiment, the culture medium comprises serum. Placental stem cells can be cultured in, for example, DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), dextrose, L-ascorbic acid, PDGF, EGF, IGF-1, and penicillin/streptomycin; DMEM-HG (high glucose) comprising 10% fetal bovine serum (FBS); DMEM-HG comprising 15% FBS; IMDM (Iscove's modified Dulbecco's medium) comprising 10% FBS, 10% horse serum, and hydrocortisone; M199 comprising 10% FBS, EGF, and heparin; α-MEM (minimal essential medium) comprising 10% FBS, GlutaMAX™ and gentamicin; DMEM comprising 10% FBS, GlutaMAX™ and gentamicin, etc. A preferred medium is DMEM-LG/MCDB-201 comprising 2% FBS, ITS, LA+BSA, dextrose, L-ascorbic acid, PDGF, EGF, and penicillin/streptomycin.

Other media in that can be used to culture placental stem cells include DMEM (high or low glucose), Eagle's basal medium, Ham's F10 medium (F10), Ham's F-12 medium (F12), Iscove's modified Dulbecco's medium, Mesenchymal Stem Cell Growth Medium (MSCGM), Liebovitz's L-15 medium, MCDB, DMIEM/F12, RPMI 1640, advanced DMEM (Gibco), DMEM/MCDB201 (Sigma), and CELL-GRO FREE.

The culture medium can be supplemented with one or more components including, for example, serum (e.g., fetal bovine serum (FBS), preferably about 2-15% (v/v); equine (horse) serum (ES); human serum (HS)); beta-mercaptoethanol (BME), preferably about 0.001% (v/v); one or more growth factors, for example, platelet-derived growth factor (PDGF), epidermal growth factor (EGF), basic fibroblast growth factor (bFGF), insulin-like growth factor-1 (IGF-1), leukemia inhibitory factor (LIF), vascular endothelial growth factor (VEGF), and erythropoietin (EPO); amino acids, including L-valine; and one or more antibiotic and/or antimycotic agents to control microbial contamination, such as, for example, penicillin G, streptomycin sulfate, amphotericin B, gentamicin, and nystatin, either alone or in combination.

4.3.9.2 Expansion and Proliferation of Placental Stem Cells

Once an isolated placental stem cell, or population of isolated stem cells (e.g., a stem cell or population of stem cells separated from at least 50% of the placental cells with which the stem cell or population of stem cells is normally associated in vivo), the stem cell or population of stem cells can be proliferated and expanded in vitro. For example, a population of placental stem cells can be cultured in tissue culture containers, e.g., dishes, flasks, multiwell plates, or the like, for a sufficient time for the stem cells to proliferate to 70-90% confluence, that is, until the stem cells and their progeny occupy 70-90% of the culturing surface area of the tissue culture container.

Placental stem cells can be seeded in culture vessels at a density that allows cell growth. For example, the cells may be seeded at low density (e.g., about 1,000 to about 5,000 cells/cm²) to high density (e.g., about 50,000 or more cells/cm²). In a preferred embodiment, the cells are cultured at about 0 to about 5 percent by volume CO₂ in air. In some preferred embodiments, the cells are cultured at about 2 to about 25 percent O₂ in air, preferably about 5 to about 20 percent O₂ in air. The cells preferably are cultured at about 25° C. to about 40° C., preferably 37° C. The cells are preferably cultured in an incubator. The culture medium can be static or agitated, for example, using a bioreactor. Placental stem cells preferably are grown under low oxidative stress (e.g., with addition of glutathione, ascorbic acid, catalase, tocopherol, N-acetylcysteine, or the like).

Once 70%-90% confluence is obtained, the cells may be passaged. For example, the cells can be enzymatically treated, e.g., trypsinized, using techniques well-known in the art, to separate them from the tissue culture surface. After removing the cells by pipetting and counting the cells, about 20,000-100,000 stem cells, preferably about 50,000 stem cells, are passaged to a new culture container containing fresh culture medium. Typically, the new medium is the same type of medium from which the stem cells were removed. Provided herein are populations of placental stem cells that have been passaged at least 1, 2, 3, 4, 5, 6, 7, 8, 9, 10, 12, 14, 16, 18, or 20 times, or more, and combinations of the same.

4.3.10 Placental Stem Cell Populations

The compositions and methods of use thereof provided herein, in certain embodiments, use populations of placental stem cells. Placental stem cell populations can be isolated directly from one or more placentas; that is, the placental stem cell population can be a population of placental cells, comprising placental stem cells, obtained from, or contained within, perfusate, or obtained from, or contained within, digestate (that is, the collection of cells obtained by enzymatic digestion of a placenta or part thereof). Isolated placental stem cells as described herein can also be cultured and expanded to produce placental stem cell populations. Populations of placental stem cells comprising placental stem cells (e.g., PDACs) can also be cultured and expanded to produce placental stem cell populations, e.g., placental stem cell population comprising PDACs, or population of PDACs.

Placental stem cell populations described herein comprise placental stem cells (e.g., PDACs) as described herein. In various embodiments, at least 10%, 20%, 30%, 40%, 50%, 60%, 70%, 80%, 90%, 95%, or 99% of the cells in an isolated placental stem cell population are placental stem cells. That is, a placental stem cell population can comprise, e.g., as much as 1%, 5%, 10%, 20%, 30%, 40%, 50%, 60%, 70%, 80%, 90% non-stem cells.

Provided herein are methods of producing isolated placental stem cell populations by, e.g., selecting placental stem cells, whether derived from enzymatic digestion or perfusion, that express particular markers and/or particular culture or morphological characteristics. In one embodiment, for example, a cell population can be produced by a method comprising selecting placental stem cells that (a) adhere to a substrate, and (b) express CD200 and do not express HLA-G; and isolating said cells from other cells to form a cell population. In another embodiment, the method of producing a cell population comprises selecting placental stem cells that (a) adhere to a substrate, and (b) express CD73, CD105, and CD200; and isolating said cells from other cells to form a cell population. In another embodiment, the method of producing a cell population comprises selecting placental stem cells that (a) adhere to a substrate and (b) express CD200 and OCT-4; and isolating said cells from other cells to form a cell population. In another embodiment, the method of producing a cell population comprises selecting placental stem cells that (a) adhere to a substrate, (b) express CD73 and CD105, and (c) facilitate the formation of one or more embryoid-like bodies in a population of placental cells comprising said stem cell when said population is cultured under conditions that allow for the formation of an embryoid-like body; and isolating said cells from other cells to form a cell population. In another embodiment, the method of producing a cell population comprises selecting placental stem cells that (a) adhere to a substrate, and (b) express CD73 and CD105, and do not express HLA-G; and isolating said cells from other cells to form a cell population. In another embodiment, the method of producing a cell population comprises selecting placental stem cells that (a) adhere to a substrate, (b) express OCT-4, and (c) facilitate the formation of one or more embryoid-like bodies in a population of placental cells comprising said stem cell when said population is cultured under conditions that allow for the formation of an embryoid-like body; and isolating said cells from other cells to form a cell population. In any of the above embodiments, the method can additionally comprise selecting placental stem cells that express ABC-p (a placenta-specific ABC transporter protein; see, e.g., Allikmets et al., Cancer Res. 58(23):5337-9 (1998)). The method can also comprise selecting cells exhibiting at least one characteristic specific to, e.g., a mesenchymal stem cell, for example, expression of CD29, expression of CD44, expression of CD90, or expression of a combination of the foregoing.

In the above embodiments, the substrate can be any surface on which culture and/or selection of cells, e.g., placental stem cells, can be accomplished. Typically, the substrate is plastic, e.g., tissue culture dish or multiwell plate plastic. Tissue culture plastic can be coated with a biomolecule, e.g., laminin or fibronectin.

Cells, e.g., placental stem cells, can be selected for a placental cell population by any means known in the art of cell selection. For example, cells can be selected using an antibody or antibodies to one or more cell surface markers, for example, in flow cytometry or FACS. Selection can be accomplished using antibodies in conjunction with magnetic beads. Antibodies that are specific for certain stem cell-related markers are known in the art. For example, antibodies to OCT-4 (Abcam, Cambridge, Mass.), CD200 (Abcam), HLA-G (Abcam), CD73 (BD Biosciences Pharmingen, San Diego, Calif.), CD105 (Abcam; BioDesign International, Saco, Me.), etc. Antibodies to other markers are also available commercially, e.g., CD34, CD38 and CD45 are available from, e.g., StemCell Technologies or BioDesign International.

The isolated placental stem cell population can comprise placental cells that are not stem cells, or cells that are not placental cells.

Isolated placental stem cell populations can be combined with one or more populations of non-stem cells or non-placental cells. For example, an isolated population of placental stem cells can be combined with blood (e.g., placental blood or umbilical cord blood), blood-derived stem cells (e.g., stem cells derived from placental blood or umbilical cord blood), populations of blood-derived nucleated cells, bone marrow-derived mesenchymal cells, bone-derived stem cell populations, crude bone marrow, adult (somatic) stem cells, populations of stem cells contained within tissue, cultured stem cells, populations of fully-differentiated cells (e.g., chondrocytes, fibroblasts, amniotic cells, osteoblasts, muscle cells, cardiac cells, etc.) and the like. Cells in an isolated placental stem cell population can be combined with a plurality of cells of another type in ratios of about 100,000,000:1, 50,000,000:1, 20,000,000:1, 10,000,000:1, 5,000,000:1, 2,000,000:1, 1,000,000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000, comparing numbers of total nucleated cells in each population. Cells in an isolated placental stem cell population can be combined with a plurality of cells of a plurality of cell types, as well.

In one, an isolated population of placental stem cells is combined with a plurality of hematopoietic stem cells. Such hematopoietic stem cells can be, for example, contained within unprocessed placental, umbilical cord blood or peripheral blood; in total nucleated cells from placental blood, umbilical cord blood or peripheral blood; in an isolated population of CD34⁺ cells from placental blood, umbilical cord blood or peripheral blood; in unprocessed bone marrow; in total nucleated cells from bone marrow; in an isolated population of CD34⁺ cells from bone marrow, or the like.

4.3.11 Preservation of Placental Stem Cells

Placental stem cells can be preserved, that is, placed under conditions that allow for long-term storage, or conditions that inhibit cell death by, e.g., apoptosis or necrosis.

Placental stem cells can be preserved using, e.g., a composition comprising an apoptosis inhibitor, necrosis inhibitor and/or an oxygen-carrying perfluorocarbon, as described in related U.S. Application Publication No. 2007/0190042, entitled “Improved Composition for Collecting and Preserving Placental cells and Methods of Using the Composition” filed on Dec. 25, 2005. In one embodiment, provided herein is a method of preserving a population of stem cells comprising contacting said population of stem cells with a stem cell collection composition comprising an inhibitor of apoptosis and an oxygen-carrying perfluorocarbon, wherein said inhibitor of apoptosis is present in an amount and for a time sufficient to reduce or prevent apoptosis in the population of stem cells, as compared to a population of stem cells not contacted with the inhibitor of apoptosis. In a specific embodiment, said inhibitor of apoptosis is a caspase inhibitor. In another specific embodiment, said inhibitor of apoptosis is a JNK inhibitor. In a more specific embodiment, said JNK inhibitor does not modulate differentiation or proliferation of said stem cells. In another embodiment, said stem cell collection composition comprises said inhibitor of apoptosis and said oxygen-carrying perfluorocarbon in separate phases. In another embodiment, said stem cell collection composition comprises said inhibitor of apoptosis and said oxygen-carrying perfluorocarbon in an emulsion. In another embodiment, the stem cell collection composition additionally comprises an emulsifier, e.g., lecithin. In another embodiment, said apoptosis inhibitor and said perfluorocarbon are between about 0° C. and about 25° C. at the time of contacting the stem cells. In another more specific embodiment, said apoptosis inhibitor and said perfluorocarbon are between about 2° C. and 10° C., or between about 2° C. and about 5° C., at the time of contacting the stem cells. In another more specific embodiment, said contacting is performed during transport of said population of stem cells. In another more specific embodiment, said contacting is performed during freezing and thawing of said population of stem cells.

In another embodiment, populations of placental stem cells can be preserved by a method comprising contacting said population of stem cells with an inhibitor of apoptosis and an organ-preserving compound, wherein said inhibitor of apoptosis is present in an amount and for a time sufficient to reduce or prevent apoptosis in the population of stem cells, as compared to a population of stem cells not contacted with the inhibitor of apoptosis. In a specific embodiment, the organ-preserving compound is UW solution (described in U.S. Pat. No. 4,798,824; also known as ViaSpan; see also Southard et al., Transplantation 49(2):251-257 (1990)) or a solution described in Stern et al., U.S. Pat. No. 5,552,267. In another embodiment, said organ-preserving compound is hydroxyethyl starch, lactobionic acid, raffinose, or a combination thereof. In another embodiment, the stem cell collection composition additionally comprises an oxygen-carrying perfluorocarbon, either in two phases or as an emulsion.

In another embodiment of the method, placental stem cells are contacted with a stem cell collection composition comprising an apoptosis inhibitor and oxygen-carrying perfluorocarbon, organ-preserving compound, or combination thereof, during perfusion. In another embodiment, said stem cells are contacted during a process of tissue disruption, e.g., enzymatic digestion. In another embodiment, placental stem cells are contacted with said stem cell collection compound after collection by perfusion, or after collection by tissue disruption, e.g., enzymatic digestion.

Typically, during placental stem cell collection, enrichment and isolation, it is preferable to minimize or eliminate cell stress due to hypoxia and mechanical stress. In another embodiment of the method, therefore, a stem cell, or population of stem cells, is exposed to a hypoxic condition during collection, enrichment or isolation for less than six hours during said preservation, wherein a hypoxic condition is a concentration of oxygen that is less than normal blood oxygen concentration. In a more specific embodiment, said population of stem cells is exposed to said hypoxic condition for less than two hours during said preservation. In another more specific embodiment, said population of stem cells is exposed to said hypoxic condition for less than one hour, or less than thirty minutes, or is not exposed to a hypoxic condition, during collection, enrichment or isolation. In another specific embodiment, said population of stem cells is not exposed to shear stress during collection, enrichment or isolation.

The placental stem cells described herein can be cryopreserved, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., C2695, C2639 or C6039 (Sigma). Cryopreservation medium preferably comprises DMSO (dimethylsulfoxide), at a concentration of, e.g., about 10% (v/v). Cryopreservation medium may comprise additional agents, for example, Plasmalyte, methylcellulose with or without glycerol. Placental stem cells are preferably cooled at about 1° C./min during cryopreservation. A preferred cryopreservation temperature is about −80° C. to about −180° C., preferably about −125° C. to about −140° C. Cryopreserved cells can be transferred to liquid nitrogen prior to thawing for use. In some embodiments, for example, once the ampoules have reached about −90° C., they are transferred to a liquid nitrogen storage area. Cryopreserved cells preferably are thawed at a temperature of about 25° C. to about 40° C., preferably to a temperature of about 37° C.

Cryopreserved immunosuppressive placental stem cell populations can comprise placental stem cells derived from a single donor, or from multiple donors. The placental stem cell population can be completely HLA-matched to an intended recipient, or partially or completely HLA-mismatched.

4.3.12 Genetically Modified Placental Stem Cells

In another aspect, provided herein are placental stem cells that are genetically modified, e.g., to produce a nucleic acid or polypeptide of interest. Genetic modification can be accomplished, e.g., using virus-based vectors including, but not limited to, non-integrating replicating vectors, e.g., papilloma virus vectors, SV40 vectors, adenoviral vectors; integrating viral vectors, e.g., retrovirus vector or adeno-associated viral vectors; or replication-defective viral vectors. Other methods of introducing DNA into cells include the use of liposomes, electroporation, a particle gun, direct DNA injection, or the like.

Stem cells can be, e.g., transformed or transfected with DNA controlled by or in operative association with, one or more appropriate expression control elements, for example, promoter or enhancer sequences, transcription terminators, polyadenylation sites, internal ribosomal entry sites. Preferably, such a DNA incorporates a selectable marker. Following the introduction of the foreign DNA, engineered stem cells can be, e.g., grown in enriched media and then switched to selective media. In one embodiment, the DNA used to engineer a placental stem cell comprises a nucleotide sequence encoding a polypeptide of interest, e.g., a cytokine, growth factor, differentiation agent, or therapeutic polypeptide.

The DNA used to engineer the stem cell can comprise any promoter known in the art to drive expression of a nucleotide sequence in mammalian cells, e.g., human cells. For example, promoters include, but are not limited to, CMV promoter/enhancer, SV40 promoter, papillomavirus promoter, Epstein-Ban virus promoter, elastin gene promoter, and the like. In a specific embodiment, the promoter is regulatable so that the nucleotide sequence is expressed only when desired. Promoters can be either inducible (e.g., those associated with metallothionein and heat shock proteins) or constitutive.

In another specific embodiment, the promoter is tissue-specific or exhibits tissue specificity. Examples of such promoters include but are not limited to: myelin basic protein gene control region (Readhead et al., 1987, Cell 48:703) (oligodendrocyte cells); elastase I gene control region (Swit et al., 1984, Cell 38:639; Ornitz et al., 1986, Cold Spring Harbor Symp. Quant. Biol. 50:399; MacDonald, 1987, Hepatology 7:425) (pancreatic acinar cells); insulin gene control region (Hanahan, 1985, Nature 315:115) (pancreatic beta cells); myosin light chain-2 gene control region (Shani, 1985, Nature 314:283) (skeletal muscle).

Placental stem cells may be engineered to “knock out” or “knock down” expression of one or more genes. The expression of a gene native to a cell can be diminished by, for example, inhibition of expression by inactivating the gene completely by, e.g., homologous recombination. In one embodiment, for example, an exon encoding an important region of the protein, or an exon 5′ to that region, is interrupted by a positive selectable marker, e.g., neo, preventing the production of normal mRNA from the target gene and resulting in inactivation of the gene. A gene may also be inactivated by creating a deletion in part of a gene or by deleting the entire gene. By using a construct with two regions of homology to the target gene that are far apart in the genome, the sequences intervening the two regions can be deleted (Mombaerts et al., 1991, Proc. Nat. Acad. Sci. U.S.A. 88:3084). Antisense, DNAzymes, small interfering RNA, and ribozyme molecules that inhibit expression of the target gene can also be used to reduce the level of target gene activity in the stem cells. For example, antisense RNA molecules which inhibit the expression of major histocompatibility gene complexes (HLA) have been shown to be most versatile with respect to immune responses. Triple helix molecules can be utilized in reducing the level of target gene activity. See, e.g., L. G. Davis et al. (eds), 1994, BASIC METHODS IN MOLECULAR BIOLOGY, 2nd ed., Appleton & Lange, Norwalk, Conn., which is incorporated herein by reference.

In a specific embodiment, placental stem cells can be genetically modified with a nucleic acid molecule comprising a nucleotide sequence encoding a polypeptide of interest, wherein expression of the polypeptide of interest is controllable by an exogenous factor, e.g., polypeptide, small organic molecule, or the like. Such a polypeptide can be a therapeutic polypeptide. In a more specific embodiment, the polypeptide of interest is IL-12 or interleukin-1 receptor antagonist (IL-1Ra). In another more specific embodiment, the polypeptide of interest is a fusion of interleukin-1 receptor antagonist and dihydrofolate reductase (DHFR), and the exogenous factor is an antifolate, e.g., methotrexate. Such a construct is useful in the engineering of placental stem cells that express IL-1Ra, or a fusion of IL-1Ra and DHFR, upon contact with methotrexate. Such a construct can be used, e.g., in the treatment of rheumatoid arthritis. In this embodiment, the fusion of IL-1Ra and DHFR is translationally upregulated upon exposure to an antifolate such as methotrexate. Therefore, in another specific embodiment, the nucleic acid used to genetically engineer a placental stem cell can comprise nucleotide sequences encoding a first polypeptide and a second polypeptide, wherein said first and second polypeptides are expressed as a fusion protein that is translationally upregulated in the presence of an exogenous factor. The polypeptide can be expressed transiently or long-term (e.g., over the course of weeks or months).

Such a nucleic acid molecule can additionally comprise a nucleotide sequence encoding a polypeptide that allows for positive selection of engineered stem cells, or allows for visualization of the engineered stem cells. In another more specific embodiment, the nucleotide sequence encodes a polypeptide that is, e.g., fluorescent under appropriate visualization conditions, e.g., luciferase (Luc). In a more specific embodiment, such a nucleic acid molecule can comprise IL-1Ra-DHFR-IRES-Luc, where IRES is an internal ribosomal entry site.

4.3.13 Immortalized Placental Stem cell Lines

Mammalian placental stem cells can be conditionally immortalized by transfection with any suitable vector containing a growth-promoting gene, that is, a gene encoding a protein that, under appropriate conditions, promotes growth of the transfected cell, such that the production and/or activity of the growth-promoting protein is regulatable by an external factor. In a preferred embodiment the growth-promoting gene is an oncogene such as, but not limited to, v-myc, N-myc, c-myc, p53, SV40 large T antigen, polyoma large T antigen, E1a adenovirus or E7 protein of human papillomavirus.

External regulation of the growth-promoting protein can be achieved by placing the growth-promoting gene under the control of an externally-regulatable promoter, e.g., a promoter the activity of which can be controlled by, for example, modifying the temperature of the transfected cells or the composition of the medium in contact with the cells. in one embodiment, a tetracycline (tet)-controlled gene expression system can be employed (see Gossen et al., Proc. Natl. Acad. Sci. USA 89:5547-5551, 1992; Hoshimaru et al., Proc. Natl. Acad. Sci. USA 93:1518-1523, 1996). In the absence of tet, a tet-controlled transactivator (tTA) within this vector strongly activates transcription from ph_{CMV*-1}, a minimal promoter from human cytomegalovirus fused to tet operator sequences. tTA is a fusion protein of the repressor (tetR) of the transposon-10-derived tet resistance operon of Escherichia coli and the acidic domain of VP16 of herpes simplex virus. Low, non-toxic concentrations of tet (e.g., 0.01-1.0 μg/mL) almost completely abolish transactivation by tTA.

In one embodiment, the vector further contains a gene encoding a selectable marker, e.g., a protein that confers drug resistance. The bacterial neomycin resistance gene (neo^{R}) is one such marker that may be employed within the methods described herein. Cells carrying neo^{R} may be selected by means known to those of ordinary skill in the art, such as the addition of, e.g., 100-200 μg/mL G418 to the growth medium.

Transfection can be achieved by any of a variety of means known to those of ordinary skill in the art including, but not limited to, retroviral infection. In general, a cell culture may be transfected by incubation with a mixture of conditioned medium collected from the producer cell line for the vector and DMEM/F12 containing N2 supplements. For example, a placental stem cell culture prepared as described above may be infected after, e.g., five days in vitro by incubation for about 20 hours in one volume of conditioned medium and two volumes of DMEM/F12 containing N2 supplements. Transfected cells carrying a selectable marker may then be selected as described above.

Following transfection, cultures are passaged onto a surface that permits proliferation, e.g., allows at least 30% of the cells to double in a 24 hour period. Preferably, the substrate is a polyornithine/laminin substrate, consisting of tissue culture plastic coated with polyornithine (10 μg/mL) and/or laminin (10 μg/mL), a polylysine/laminin substrate or a surface treated with fibronectin. Cultures are then fed every 3-4 days with growth medium, which may or may not be supplemented with one or more proliferation-enhancing factors. Proliferation-enhancing factors may be added to the growth medium when cultures are less than 50% confluent.

The conditionally-immortalized placental stem cell lines can be passaged using standard techniques, such as by trypsinization, when 80-95% confluent. Up to approximately the twentieth passage, it is, in some embodiments, beneficial to maintain selection (by, for example, the addition of G418 for cells containing a neomycin resistance gene). Cells may also be frozen in liquid nitrogen for long-term storage.

Clonal cell lines can be isolated from a conditionally-immortalized human placental stem cell line prepared as described above. In general, such clonal cell lines may be isolated using standard techniques, such as by limit dilution or using cloning rings, and expanded. Clonal cell lines may generally be fed and passaged as described above.

Conditionally-immortalized human placental stem cell lines, which may, but need not, be clonal, may generally be induced to differentiate by suppressing the production and/or activity of the growth-promoting protein under culture conditions that facilitate differentiation. For example, if the gene encoding the growth-promoting protein is under the control of an externally-regulatable promoter, the conditions, e.g., temperature or composition of medium, may be modified to suppress transcription of the growth-promoting gene. For the tetracycline-controlled gene expression system discussed above, differentiation can be achieved by the addition of tetracycline to suppress transcription of the growth-promoting gene. In general, 1 μg/mL tetracycline for 4-5 days is sufficient to initiate differentiation. To promote further differentiation, additional agents may be included in the growth medium.

### 4.4 Pharmaceutical Compositions

Also provided herein are pharmaceutical compositions that comprise combination compositions described herein, and a pharmaceutically-acceptable carrier.

In accordance with this embodiment, the combination compositions described herein may be formulated as an injectable (e.g., WO 96/39101, incorporated herein by reference in its entirety). In another embodiment, the combination compositions may be formulated using polymerizable or cross linking hydrogels as described, e.g., in U.S. Pat. Nos. 5,709,854; 5,516,532; 5,654,381.

In another embodiment, each component of the combination composition, i.e., placental stem cells and platelet rich plasma, respectively, may be maintained prior to administration to an individual, as separate pharmaceutical compositions to be administered sequentially or jointly to create the combination composition in vivo. Each component may be stored and/or used in a separate container, e.g., one bag (e.g., blood storage bag from Baxter, Becton-Dickinson, Medcep, National Hospital Products, Terumo, etc.) or separate syringe, which contains a single type of cell or cell population. In a specific embodiment, platelet rich plasma, are contained in one bag, and placental perfusate, or placental stem cells from placental perfusate, are contained in a second bag.

A population of placental stem cells can be enriched. In a specific embodiment, a population of cells comprising placental stem cells is enriched by removal of red blood cells and/or granulocytes according to standard methods, so that the remaining population of nucleated cells is enriched for placental stem cells relative to other cell types in placental perfusate. Such an enriched population of placental stem cells may be used unfrozen, or may be frozen for later use. If the population of cells is to be frozen, a standard cryopreservative (e.g., DMSO, glycerol, EPILIFE™ Cell Freezing Medium (Cascade Biologics)) is added to the enriched population of cells before it is frozen.

The pharmaceutical compositions may comprise one or more

agents that induce cell differentiation. In certain embodiments, an agent that induces differentiation includes, but is not limited to, Ca²⁺, EGF, α-FGF, β-FGF, PDGF, keratinocyte growth factor (KGF), TGF-β, cytokines (e.g., IL-1α, IL-1β, IFN-γ, TFN), retinoic acid, transferrin, hormones (e.g., androgen, estrogen, insulin, prolactin, triiodothyroxine, hydrocortisone, dexamethasone), sodium butyrate, TPA, DMSO, NMF, DMF, matrix elements (e.g., collagen, laminin, heparan sulfate, MATRIGEL™), or combinations thereof.

In another embodiment, the pharmaceutical composition may comprise one or more agents that suppress cellular differentiation. In certain embodiments, an agent that suppresses differentiation includes, but is not limited to, human Delta-1 and human Serrate-1 polypeptides (see, Sakano et al., U.S. Pat. No. 6,337,387), leukemia inhibitory factor (LIF), stem cell factor, or combinations thereof.

The pharmaceutical compositions provided herein may be treated prior to administration to an individual with a compound that modulates the activity of TNF-α. Such compounds are disclosed in detail in, e.g., U.S. Application Publication No. 2003/0235909, which disclosure is incorporated herein in its entirety. Preferred compounds are referred to as IMiDs (immunomodulatory compounds) and SelCIDs (Selective Cytokine Inhibitory Drugs), and particularly preferred compounds are available under the trade names ACTIMID™, REVIMID™ and REVLIMID™.

4.4.1 Matrices Comprising Combination Compositions

Further provided herein are matrices, hydrogels, scaffolds, and the like that comprise placental stem cells and platelet rich plasma.

Placental stem cells or platelet rich plasma alone, e.g., prior to subsequent addition of the other component of the combination composition in vivo, or placental stem cells combined with platelet rich plasma, can be seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. Preferred placental biomaterials on which placental stem cells can be seeded are described in Hariri, U.S. Application Publication No. 2004/0048796.

Placental stem cells or platelet rich plasma alone, e.g., prior to subsequent addition of the other component of the combination composition in vivo, or placental stem cells combined with platelet rich plasma, can be suspended in a hydrogel solution suitable for, e.g., injection. Suitable hydrogels for such compositions include self-assembling peptides, such as RAD16. In one embodiment, a hydrogel solution comprising the one or both components of the combination composition can be allowed to harden, for instance in a mold, to form a matrix for implantation. Placental stem cells in such a matrix can also be cultured so that the cells are mitotically expanded prior to implantation. The hydrogel is, e.g., an organic polymer (natural or synthetic) that is cross-linked via covalent, ionic, or hydrogen bonds to create a three-dimensional open-lattice structure that entraps water molecules to form a gel. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In some embodiments, the formulation comprises an in situ polymerizable gel (see., e.g., U.S. Patent Application Publication 2002/0022676; Anseth et al., J. Control Release, 78(1-3):199-209 (2002); Wang et al., Biomaterials, 24(22):3969-80 (2003).

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

Placental stem cells or platelet rich plasma alone, e.g., prior to subsequent addition of the other component of the combination composition in vivo, or placental stem cells combined with platelet rich plasma, can be seeded onto a three-dimensional framework or scaffold and implanted in vivo. Such a framework can be implanted in combination with any one or more growth factors, cells, drugs or other components that stimulate tissue formation or otherwise enhance or improve the practice of the methods of treatment described elsewhere herein.

Examples of scaffolds that can be used in the methods of treatment described herein include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(ε-caprolactone)/poly(glycolic acid) (PCL/PGA) copolymer, formed by processes such as freeze-drying, or lyophilization (see, e.g., U.S. Pat. No. 6,355,699), can also be used as scaffolds. Other scaffolds may be comprised of oxidized cellulose or oxidized regenerated cellulose.

In another embodiment, the scaffold is, or comprises, a nanofibrous scaffold, e.g., an electrospun nanofibrous scaffold. In a more specific embodiment, said nanofibrous scaffold comprises poly(L-lactic acid) (PLLA), type I collagen, a copolymer of vinylidene fluoride and trifluoroethylnee (PVDF-TrFE), poly(-caprolactone), poly(L-lactide-co-ε-caprolactone) [P(LLA-CL)] (e.g., 75:25), and/or a copolymer of poly(3-hydroxybutyrate-co-3-hydroxyvalerate) (PHBV) and type I collagen. In another more specific embodiment, said scaffold promotes the differentiation of placental stem cells into chondrocytes. Methods of producing nanofibrous scaffolds, e.g., electrospun nanofibrous scaffolds, are known in the art. See, e.g., Xu et al., Tissue Engineering 10(7):1160-1168 (2004); Xu et al., Biomaterials 25:877-886 (20040; Meng et al., J. Biomaterials Sci., Polymer Edition 18(1):81-94 (2007).

Placental stem cells or platelet rich plasma alone, e.g., prior to subsequent addition of the other component of the combination composition in vivo, or placental stem cells combined with platelet rich plasma, can also be seeded onto, or contacted with, a physiologically-acceptable ceramic material including, but not limited to, mono-, di-, tri-, alpha-tri-, beta-tri-, and tetra-calcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include SURGIBONE® (CanMedica Corp., Canada), ENDOBON® (Merck Biomaterial France, France), CEROS® (Mathys, AG, Bettlach, Switzerland), and mineralized collagen bone grafting products such as HEALOS™ (DePuy, Inc., Raynham, Mass.) and VITOSS®, RHAKOSS™, and CORTOSS® (Orthovita, Malvern, Pa.). The framework can be a mixture, blend or composite of natural and/or synthetic materials.

In another embodiment, placental stem cells or platelet rich plasma alone, e.g., prior to subsequent addition of the other component of the combination composition in vivo, or placental stem cells combined with platelet rich plasma, can be seeded onto, or contacted with, a felt, which can be, e.g., composed of a multifilament yarn made from a bioabsorbable material such as PGA, PLA, PCL copolymers or blends, or hyaluronic acid.

Placental stem cells or platelet rich plasma alone, e.g., prior to subsequent addition of the other component of the combination composition in vivo, or placental stem cells combined with platelet rich plasma, can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inoculation of the immunosuppressive placental stem cells in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with placental stem cells.

In particular embodiments, the combination compositions comprising placental stem cells and platelet rich plasma provided herein are not seeded on a matrix, hydrogels, scaffolds, and the like prior to transplantation in an individual in need of said combination composition. In another particular embodiment, the combination compositions do not comprise an implantable bone substitute when transplanted in an individual in need of said combination composition.

### 4.5 Methods of Transplanting Compositions Comprising Placental Stem Cells and Platelet Rich Plasma

In some embodiments, an individual is contacted with a combination composition comprising placental stem cells and platelet rich plasma as provided herein. In a specific embodiment, said contacting is the introduction, e.g., transplantation, of said combination composition into said individual. Thus, the method of combining placental stem cells with platelet rich plasma may be performed as a first step in a procedure for introducing the combination composition into any individual needing stem cells. Such a procedure can comprise use of pharmaceutical compositions comprising the combination compositions, as described above. Alternatively, each component of the combination composition can be introduced, e.g., transplanted into said individual serially. For example, platelet rich plasma may be administered to the individual in a first step, near the area where the pathogenesis is present, to form a stable hydrogel in vivo. In a second step, placental stem cells may be administered, e.g., injected into the formed hydrogel.

In a specific embodiment, a population of placental stem cells of the compositions provided herein is combined with platelet rich plasma prior to administration to an individual in need thereof in a ratio that results in prolonged localization of the placental stem cells at the site of injection or implantation, relative to administration of placental stem cells not combined with platelet rich plasma. In another specific embodiment, a population of placental stem cells of the compositions provided herein is combined with platelet rich plasma during, or simultaneously with, administration to a patient in need thereof, in an optimum ratio, that results in prolonged localization of the placental stem cells at the site of injection or implantation, relative to administration of placental stem cells not combined with platelet rich plasma. In another specific embodiment, a population of placental stem cells and a platelet rich plasma are administered sequentially to a patient in need thereof to a final optimum ratio. In one embodiment, the population of placental stem cells is administered first and the platelet rich plasma is administered second. In another embodiment, the platelet rich plasma is administered first and the population of placental stem cells is administered second.

In a specific embodiment, said combination composition comprising placental stem cells and platelet rich plasma is contained within one bag or container. In another embodiment, a population of placental stem cells, and platelet rich plasma, contained within separate bags or containers is used in a transplantation procedure. In certain embodiments, placental stem cells and platelet rich plasma contained in two separate bags may be mixed prior, in particular immediately prior, to or at the time of administration to a patient in need thereof.

The combining, i.e., mixing of placental stem cells with platelet rich plasma to obtain the combination compositions provided herein is performed gently so as to not activate the platelets within the PRP.

In particular embodiments, the placental stem cells and platelet rich plasma are provided in separate chambers of a 2-chamber syringe and reconstituted in the syringe prior to administration, e.g., injection into the individual.

Combination compositions of placental stem cells, and platelet rich plasma may be mixed, prior to transplantation, by any medically-acceptable means. In one embodiment, the two components are physically mixed. In another embodiment of the method, said placental stem cells and platelet rich plasma are mixed immediately prior to (i.e., within 1, 2, 3, 4, 5, 7, or 30 minutes of) administration to said individual. In another embodiment, said placental stem cells and platelet rich plasma are mixed at a point in time more than five minutes prior to administration to said individual. In another embodiment of the method, the placental stem cells, and/or platelet rich plasma, are cryopreserved and thawed prior to administration to said individual. In another embodiment, said placental stem cells and platelet rich plasma are mixed to form a combination composition at a point in time more than 1, 2, 3, 4, 5, 6, 7, 8, 9, 10, 11, 12, 13, 14, 15, 16, 17, 18, 19, 20, 21, 22, 23, or 24 hours prior to administration to said individual, wherein either or both of said placental stem cells and platelet rich plasma have been cryopreserved and thawed prior to said administration. In another embodiment, the combination composition may be administered more than once.

In some embodiments, the platelet rich plasma component of the combination composition, when administered separately from the placental stem cell component, can be adinistered as a liquid, a solid, a semi-solid (e.g., a gel), or a combination thereof. In these embodiments, when the platelet rich plasma is delivered as a liquid, it may comprise a solution, an emulsion, a suspension, etc.

In some embodiments, a platelet rich plasma semi-solid or gel may be prepared by adding an agent to the platelet rich plasma, alone or combined with placental stem cells, e.g., to better preserve the position of the placental stem cells once the combination composition is delivered to the target tissue. For example, the platelet rich plasma, alone or in combination with placental stem cells, may include, for example, collagen, cyanoacrylate, adhesives that cure upon injection into tissue, liquids that solidify or gel after injection into tissue, suture material, agar, gelatin, light-activated dental composite, other dental composites, silk-elastin polymers, Matrigel™, gelatinous protein mixture (BD Biosciences), hydrogels and/or other suitable biopolymers. In certain other embodiments, a clotting agent (e.g., thrombin and/or calcium) may be added to the PRP alone or combined with placental stem cells. Alternatively, the clotting agent may be delivered to the target tissue before or after platelet rich plasma, alone or in combination with placental stem cells, has been delivered to the target tissue to cause the platelet rich plasma to gel. In other embodiments, no clotting agents are added to the platelet rich plasma or to the combination composition comprising platelet rich plasma. In particular embodiments, the composition comprising placental stem cells combined with platelet rich plasma, provided herein, does not comprise, and does not require a clotting agent (e.g., thrombin and/or calcium) to effect prolonged localization of the placental stem cells at the site of injection or implantation, relative to administration of placental stem cells not combined with platelet rich plasma. For example, platelet rich plasma, alone or in combination with placental stem cells, may harden or gel in response to one or more environmental or chemical factors such as temperature, pH, proteins, etc., without the addition of a clotting agent.

In another embodiment, the placental stem cells contained within the combination composition are preconditioned prior to transplantation. In a preferred embodiment, preconditioning comprises storing the cells in a gas-permeable container generally for a period of time at about −5° C. to about 23° C., about 0° C. to about 10° C., or preferably about 4° C. to about 5° C. The cells may be stored between 18 hours and 21 days, between 48 hours and 10 days, preferably between 3-5 days. The cells may be cryopreserved prior to preconditioning or, may be preconditioned immediately prior to administration.

In some embodiments, the placental stem cells may be differentiated prior to introduction of the combination composition to an individual in need of stem cells. For example, for introduction for the purpose of neural, epithelial or vascular engraftment, the stem cells may be differentiated to cells in the neurogenic, epithelial or vascular lineage, respectively. The combination of differentiated stem cells and platelet rich plasma is encompassed within the phrase “combination composition.” In certain embodiments of the invention, the method of transplantation of a combination composition provided herein comprises (a) induction of differentiation of placental stem cells, (b) mixing the placental stem cells with platelet rich plasma to form a combination composition, and (c) administration of the combination composition to an individual in need thereof.

The combination compositions provided herein, or each component of the combination composition, may be transplanted into a patient in any pharmaceutically or medically acceptable manner, including by surgical implantation or injection, e.g., intraarticular injection, intramuscular injection, intraperitoneal injection, intraocular injection, direct injection into a particular tissue. The site of delivery of the combination composition is typically at or near the site of pathogenesis, e.g., tissue damage. The site of tissue damage can be determined by well-established methods including medical imaging, nerve conduction studies, injections of dyes, patient feedback, or a combination thereof. The particular imaging method used may be determined based on the tissue type. Commonly used imaging methods include, but are not limited to MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging and ultrasound techniques. The patient may also assist in locating the site of tissue injury or damage by pointing out areas of particular pain and/or discomfort. The PRP composition may be delivered minimally invasively and/or surgically. For example, to deliver a PRP composition to ischemic tissue, a physician may use one of a variety of access techniques, including but not limited to, surgical (e.g., sternotomy, thoracotomy, mini-thoracotomy, sub-xiphoidal) approaches, endoscopic approaches (e.g., intercostal and transxiphoidal) and percutaneous (e.g., transvascular) approaches.

The combination composition may comprise, or be suspended in, any pharmaceutically-acceptable carrier. The combination composition may be carried, stored, or transported in any pharmaceutically or medically acceptable container, for example, a blood bag, transfer bag, plastic tube or vial.

After transplantation, engraftment in a human recipient may be assessed using, e.g., nucleic acid or protein detection or analytical methods. For example, the polymerase chain reaction (PCR), STR, SSCP, RFLP analysis, AFLP analysis, and the like, may be used to identify engrafted cell-specific nucleotide sequences in a tissue sample from the recipient. Such nucleic acid detection and analysis methods are well-known in the art. In one embodiment, engraftment may be determined by the appearance of engrafted cell-specific nucleic acids in a tissue sample from a recipient, which are distinguishable from background. The tissue sample analyzed may be, for example, a biopsy (e.g., bone marrow aspirate) or a blood sample.

In one embodiment, a sample of peripheral blood is taken from a patient immediately prior to a medical procedure, e.g., myeloablation. After the procedure, a combination composition comprising placental stem cells and platelet rich plasma is administered to the patient. At least once post-administration, a second sample of peripheral blood is taken. An STR profile is obtained for both samples, e.g., using PCR primers for markers (alleles) available from, e.g., LabCorp (Laboratory Corporation of America). A difference in the number or characteristics of the markers (alleles) post-administration indicates that engraftment has taken place.

Engraftment can also be demonstrated by detection of re-emergence of neutrophils.

In another example, engrafted cell-specific markers may be detected in a tissue sample from the recipient using antibodies directed to markers specific to either the transplanted stem cells, or cells into which the transplanted stem cells would be expected to differentiate. In one embodiment, engraftment of a combination of placental stem cells and platelet rich plasma may be assessed by FACS analysis to determine the presence of CD45⁺, CD19⁺, CD33⁺, CD7⁺ and/or CD3⁺ cells by adding the appropriate antibody and allowing binding; washing (e.g., with PBS); fixing the cells (e.g., with 1% paraformaldehyde); and analyzing on an appropriate FACS apparatus (e.g., a FACSCalibur flow cytometer (Becton Dickinson)). In another embodiment, engraftment of a combination of placental stem cells and platelet rich plasma may be assessed by FACS analysis to determine the presence of CD200⁺ or HLA-G⁺ cells. Where placental stem cells and/or platelet rich plasma are from an individual of a different sex than a recipient, e.g., male donor and female recipient, engraftment can be determined by detection of sex-specific markers, e.g., Y-chromosome-specific markers. Placental stem cells may also be genetically modified to express a unique marker or nucleic acid sequence that facilitates identification, e.g., an RFLP marker, expression of β-galactosidase or green fluorescent protein, or the like.

The degree of engraftment may be assessed by any means known in the art. In one embodiment, the degree of engraftment is assessed by a grading system as follows, which uses a thin section of fixed and antibody-bound tissue from the transplant recipient. In this example grading system, engraftment is graded as follows: 0=no positive cells (that is, no cells bound by an antibody specific to an engrafted cell); 0.5=one or two positive cells, perhaps positive, but difficult to differentiate from background or non-specific staining; 1=2-20 scattered positive cells; 2=approximately 20-100 scattered or clustered positive cells throughout the tissue; 3=more than 100 positive cells comprising less than 50% of the tissue; 4=more than 50% of cells are positive. In specific embodiments, engraftment is determined where greater than 0.5%, 1%, 2%, 3%, 4%, 5%, 7.5%, 10%, 15%, 20% or greater of the cells are positively stained.

### 4.6 Methods of Treatment Using Compositions Comprising Placental Stem Cells and Platelet Rich Plasma

The compositions comprising placental stem cells and platelet rich plasma provided herein can be used to treat individuals exhibiting a variety of disease states or conditions that would benefit from reduced inflammation, promotion of angiogenesis, and enhanced healing. Examples of such disease states or conditions include, but are not limited to: repetitive use injuries, such as lateral epicondylitis (tennis elbow) and carpal tunnel syndrome; sports injuries, such as torn ligaments and tendons, torn rotator cuffs and meniscal tears; degenerative joint conditions such as osteoarthritis of the hip, knee, shoulder, elbow; disease of or trauma to a joint; disease states or conditions characterized by a disruption of blood flow in the peripheral vasculature, such as peripheral arterial disease (PAD), e.g., critical limb ischemia (CLI); neuropathic pain; dermatological conditions, e.g., for the treatment of wounds (external and internal), acute and chronic wounds, e.g., various ulcers, congenital wounds, burns, and skin conditions, e.g., skin lesions; and bone related uses and the treatment of orthopedic defects, e.g., disc herniation and degenerative disc disease. Thus, in another aspect, provided herein is a method of treating an individual suffering from a disease or condition that would benefit from reduced inflammation, promotion of angiogenesis, and enhanced healing, comprising administering a therapeutically effective amount of a composition comprising placental stem cells and platelet rich plasma, as described herein, to said individual in an amount and for a time sufficient for detectable improvement of said disease or condition.

In certain embodiments, the individual is an animal, preferably a mammal, more preferably a non-human primate. In certain embodiments, the individual is a human patient. The individual can be a male or female subject. In certain embodiments, the subject is a non-human animal, such as, for instance, a cow, sheep, goat, horse, dog, cat, rabbit, rat or mouse.

Methods for the treatment of such individuals, and for the administration of such compositions comprising placental stem cells and platelet rich plasma are discussed in detail below.

4.6.1 Treatment of Vascular Conditions

In one aspect, provided herein are methods for treating an individual having a vascular disease or cardiac medical condition comprising administering to said individual a therapeutically-effective amount of a composition comprising placental stem cells and platelet-rich plasma. In a specific embodiment, the method comprises evaluating the individual for one or more indicia of improvement in vascular or cardiac function.

In one embodiment, the medical condition is a cardiomyopathy. In specific embodiments, the cardiomyopathy is either idiopathic or a cardiomyopathy with a known cause. In other specific embodiments, the cardiomyopathy is either ischemic or nonischemic in nature. In another embodiments, the vascular disease or cardiac medical condition comprises one or more of angioplasty, aneurysm, angina (angina pectoris), aortic stenosis, aortitis, arrhythmias, arteriosclerosis, arteritis, asymmetric septal hypertrophy (ASH), atherosclerosis, atrial fibrillation and flutter, bacterial endocarditis, Barlow's Syndrome (mitral valve prolapse), bradycardia, Buerger's Disease (thromboangiitis obliterans), cardiomegaly, cardiomyopathy, carditis, carotid artery disease, coarctation of the aorta, congenital heart diseases (congenital heart defects), congestive heart failure (heart failure), coronary artery disease, Eisenmenger's Syndrome, embolism, endocarditis, erythromelalgia, fibrillation, fibromuscular dysplasia, heart block, heart murmur, hypertension, hypotension, idiopathic infantile arterial calcification, Kawasaki Disease (mucocutaneous lymph node syndrome, mucocutaneous lymph node disease, infantile polyarteritis), metabolic syndrome, microvascular angina, myocardial infarction (heart attacks), myocarditis, paroxysmal atrial tachycardia (PAT), periarteritis nodosa (polyarteritis, polyarteritis nodosa), pericarditis, diabetic vasculopathy, phlebitis, pulmonary valve stenosis (pulmonic stenosis), Raynaud's Disease, renal artery stenosis, renovascular hypertension, rheumatic heart disease, septal defects, silent ischemia, syndrome X, tachycardia, Takayasu's Arteritis, Tetralogy of Fallot, transposition of the great vessels, tricuspid atresia, truncus arteriosus, valvular heart disease, varicose ulcers, varicose veins, vasculitis, ventricular septal defect, Wolff-Parkinson-White Syndrome, or endocardial cushion defect.

In another specific embodiment, the vascular disease is peripheral vascular disease, e.g., critical limb ischemia (acute limb ischemia).

In other embodiments, the vascular disease or cardiac medical condition comprises one or more of acute rheumatic fever, acute rheumatic pericarditis, acute rheumatic endocarditis, acute rheumatic myocarditis, chronic rheumatic heart diseases, diseases of the mitral valve, mitral stenosis, rheumatic mitral insufficiency, diseases of aortic valve, diseases of other endocardial structures, ischemic heart disease (acute and subacute), angina pectoris, diseases of pulmonary circulation (acute pulmonary heart disease, pulmonary embolism, chronic pulmonary heart disease), kyphoscoliotic heart disease, myocarditis, endocarditis, endomyocardial fibrosis, endocardial fibroelastosis, atrioventricular block, cardiac dysrhythmias, myocardial degeneration, diseases of the circulatory system including cerebrovascular disease, occlusion and stenosis of precerebral arteries, occlusion of cerebral arteries, diseases of arteries, arterioles and capillaries (atherosclerosis, aneurysm), or diseases of veins and lymphatic vessels.

In one embodiment, treatment comprises treatment of a patient with a cardiomyopathy with a therapeutic composition comprising placental stem cells and platelet-rich plasma. In other preferred embodiments, the individual experiences benefits from the therapy, for example from the ability of the cells to support the growth of other cells, including stem cells or progenitor cells present in the heart, from the tissue ingrowth or vascularization of the tissue, and from the presence of beneficial cellular factors, chemokines, cytokines and the like, but the cells do not integrate or multiply in the patient. In another embodiment, the individual benefits from the therapeutic treatment with the cells, but the cells do not survive for a prolonged period in the patient. In one embodiment, the cells gradually decline in number, viability or biochemical activity. In other embodiments, the decline in cells may be preceded by a period of activity, for example growth, division, or biochemical activity. In other embodiments, senescent, nonviable or even dead cells are able to have a beneficial therapeutic effect.

In another embodiment, improvement in said individual having a vascular disease or cardiac medical condition, wherein the individual has been administered the compositions comprising placental stem cells and platelet-rich plasma as provided herein, can be assessed or demonstrated by detectable improvement in one or more, indicia of cardiac function, for example, demonstration of detectable improvement in one or more of chest cardiac output (CO), cardiac index (CI), pulmonary artery wedge pressures (PAWP), and cardiac index (CI), % fractional shortening (% FS), ejection fraction (EF), left ventricular ejection fraction (LVEF); left ventricular end diastolic diameter (LVEDD), left ventricular end systolic diameter (LVESD), contractility (e.g. dP/dt), pressure-volume loops, measurements of cardiac work, an increase in atrial or ventricular functioning; an increase in pumping efficiency, a decrease in the rate of loss of pumping efficiency, a decrease in loss of hemodynamic functioning; and a decrease in complications associated with cardiomyopathy, as compared to the individual prior to administration of said compositions.

Improvement in an individual receiving the therapeutic compositions provided herein can also be assessed by subjective metrics, e.g., the individual's self-assessment about his or her state of health following administration.

Success of administration of the compositions provided herein is not, in certain embodiments, based on survival in the individual of the administered placental stem cells. Success is, instead, based on one or more metrics of improvement in cardiac or circulatory health, as noted above. Thus, the cells need not integrate into the patient's heart, or into blood vessels.

In certain embodiments, the methods of treatment provided herein comprise inducing the therapeutic placental stem cells of the compositions provided herein to differentiate along a mesenchymal lineage, e.g., towards a cardiomyogenic, angiogenic or vasculogenic phenotype, or into cells such as myocytes, cardiomyocytes, endothelial cells, myocardial cells, epicardial cells, vascular endothelial cells, smooth muscle cells (e.g. vascular smooth muscle cells).

Administration of compositions comprising placental stem cells and platelet-rich plasma as provided herein, to an individual in need thereof, can be accomplished, e.g., by transplantation, implantation (e.g., of the cells themselves or the cells as part of a matrix-cell combination), injection (e.g., directly to the site of the disease or condition, for example, directly to an ischemic site in the heart of an individual who has had a myocardial infarction), infusion, delivery via catheter, or any other means known in the art for providing cell therapy.

In one embodiment, the therapeutic cell compositions are provided to an individual in need thereof, for example, by injection into one or more sites in the individual. In a specific embodiment, the therapeutic cell compositions are provided by intracardiac injection, e.g., to an ischemic area in the heart. In other specific embodiments, the compositions are injected onto the surface of the heart, into an adjacent area, or even to a more remote area. In preferred embodiments, the cells can home to the diseased or injured area.

An individual having a disease or condition of the coronary or vascular systems can be administered a composition comprising placental stem cells and platelet rich plasma at any time the cells would be therapeutically beneficial. In certain embodiments, for example, the composition comprising placental stem cells and platelet rich plasma are administered within 1, 2, 3, 4, 5, 6, 7, 8, 9, 10, 11, 12, 13, 14, 15, 16, 17, 18, 19, 20, 21, 22, 23, or 24 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of the myocardial infarction. Administration proximal in time to a myocardial infarction, e.g., within 1-3 or 1-7 days, is preferable to administration distal in time, e.g., after 3 or 7 days after a myocardial infarction. In other embodiments, the composition is administered within 1, 2, 3, 4, 5, 6, 7, 8, 9, 10, 11, 12, 13, 14, 15, 16, 17, 18, 19, 20, 21, 22, 23, or 24 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of initial diagnosis of the disease or condition.

Also provided herein are kits for use in the treatment of myocardial infarction. The kits provide the therapeutic cell composition which can be prepared in a pharmaceutically acceptable form, for example by mixing with a pharmaceutically acceptable carrier, and an applicator, along with instructions for use. Ideally the kit can be used in the field, for example in a physician's office, or by an emergency care provider to be applied to a patient diagnosed as having had a myocardial infarction or similar cardiac event.

In specific embodiments of the methods of treatment provided herein, the compositons comprising placental stem cells and platelet rich plasma are administered with stem cells (that is, stem cells that are not placental stem cells), myoblasts, myocytes, cardiomyoblasts, cardiomyocytes, or progenitors of myoblasts, myocytes, cardiomyoblasts, and/or cardiomyocytes.

In a specific embodiment, the methods of treatment provided herein comprise administering a therapeutic composition comprising placental stem cells and platelet-rich plasma as provided herein, to a patient with a disease of the heart or circulatory system; and evaluating the patient for improvements in cardiac function, wherein the therapeutic composition is administered as a matrix-cell complex. In certain embodiments, the matrix is a scaffold, preferably bioabsorbable, comprising at least the cells or the platelet rich plasma.

To this end, provided herein are populations of placental stem cells incubated in the presence of one or more factors which stimulate stem or progenitor cell differentiation along a cardiogenic, angiogenic, hemangiogenic, or vasculogenic pathway. Such factors are known in the art; determination of suitable conditions for differentiation can be accomplished with routine experimentation. Such factors include, but are not limited to factors, such as growth factors, chemokines, cytokines, cellular products, demethylating agents, and other stimuli which are now known or later determined to stimulate differentiation, for example of stem cells, along cardiogenic, angiogenic, hemangiogenic, or vasculogenic pathways or lineages.

Placental stem cells may be differentiated along cardiogenic, angiogenic, hemangiogenic, or vasculogenic pathways or lineages by culture of the cells in the presence of factors comprising at least one of a demethylation agent, a BMP, FGF, Wnt factor protein, Hedgehog, and/or anti-Wnt factors.

Inclusion of demethylation agents tends to allow the cells to differentiate along mesenchymal lines, toward a cardiomyogenic pathway. Differentiation can be determined by, for example, expression of at least one of cardiomyosin, skeletal myosin, or GATA4; or by the acquisition of a beating rhythm, spontaneous or otherwise induced; or by the ability to integrate at least partially into a patient's cardiac muscle without inducing arrhythmias. Demethylation agents that can be used to initiate such differentiation include, but are not limited to, 5-azacytidine, 5-aza-2′-deoxycytidine, dimethylsulfoxide, chelerythrine chloride, retinoic acid or salts thereof, 2-amino-4-(ethylthio)butyric acid, procainamide, and procaine.

In certain embodiments herein, cells induced with one or more factors as identified above may become cardiomyogenic, angiogenic, hemangiogenic, or vasculogenic cells, or progenitors. In one embodiment, at least some of the cells can integrate at least partially into a recipient's cardiovascular system, including but not limited to heart muscle, vascular and other structures of the heart, cardiac or peripheral blood vessels, and the like. In certain other embodiments, the differentiated placental stem cells differentiate into cells acquiring two or more of the indicia of cardiomyogenic cells or their progenitors, and able to partially or fully integrate into a recipient's heart or vasculature. In specific embodiments, the cells, which administered to an individual, result in no increase in arrhythmias, heart defects, blood vessel defects or other anomalies of the individual's circulatory system or health. In certain embodiments, the placental stem cells act to promote the differentiation of stem cells naturally present in the patient's cardiac muscle, blood vessels, blood and the like to themselves differentiate into for example, cardiomyocytes, or at least along cardiomyogenic, angiogenic, hemangiogenic, or vasculogenic lines.

Placental stem cells, and populations of such cells, can be provided therapeutically or prophylactically in the compositions described herein to an individual, e.g., an individual having a disease, disorder or condition of, or affecting, the heart or circulatory system. Such diseases, disorders or conditions can include congestive heart failure due to atherosclerosis, cardiomyopathy, or cardiac injury, e.g., an ischemic injury, such as from myocardial infarction or wound (acute or chronic).

In certain embodiments, the individual is administered a therapeutically effective amount of a composition comprising placental stem cells and platelet-rich plasma as provided herein. In a specific embodiment, the population comprises about 50% placental stem cells. In another specific embodiment, the population is a substantially homogeneous population of placental stem cells. In other embodiments the population comprises at least about 5%, 10%, 20%, 25%, 30%, 33%, 40%, 60%, 66%, 70%, 75%, 80%, or 90% placental stem cells.

The composition comprising placental stem cells and platelet rich plasma may be administered to an individual in the form of a therapeutic composition comprising the cells, PRP, and another therapeutic agent, such as insulin-like growth factor (IGF), platelet-derived growth factor (PDGF), epidermal growth factor (EGF), fibroblast growth factor (FGF), vascular endothelial growth factor (VEGF), hepatocyte growth factor (HGF), IL-8, an antithrombogenic agent (e.g., heparin, heparin derivatives, urokinase, and PPack (dextrophenylalanine proline arginine chloromethylketone); antithrombin compounds, platelet receptor antagonists, anti-thrombin antibodies, anti-platelet receptor antibodies, aspirin, dipyridamole, protamine, hirudin, prostaglandin inhibitors, and/or platelet inhibitors), an antiapoptotic agent (e.g., EPO, EPO derivatives and analogs, and their salts, TPO, IGF-I, IGF-II, hepatocyte growth factor (HGF), or caspase inhibitors), an anti-inflammatory agent (e.g., P38 MAP kinase inhibitors, statins, IL-6 and IL-1 inhibitors, Pemirolast, Tranilast, Remicade, Sirolimus, nonsteroidal anti-inflammatory compounds, for example, acetylsalicylic acid, ibuprofen, Tepoxalin, Tolmetin, or Suprofen), an immunosuppressive or immunomodulatory agent (e.g., calcineurin inhibitors, for example cyclosporine, Tacrolimus, mTOR inhibitors such as Sirolimus or Everolimus; anti-proliferatives such as azathioprine and mycophenolate mofetil; corticosteroids, e.g., prednisolone or hydrocortisone; antibodies such as monoclonal anti-IL-2Rα receptor antibodies, Basiliximab, Daclizuma, polyclonal anti-T-cell antibodies such as anti-thymocyte globulin (ATG), anti-lymphocyte globulin (ALG), and the monoclonal anti-T cell antibody OKT3, or adherent placental stem cells as described in U.S. Pat. No. 7,468,276, and U.S. Patent Application Publication No. and 2007/0275362, the disclosures of which are incorporated herein by reference in their entireties), and/or an antioxidant (e.g., probucol; vitamins A, C, and E, coenzyme Q-10, glutathione, L cysteine, N-acetylcysteine, or antioxidant derivative, analogs or salts of the foregoing). In certain embodiments, therapeutic compositions comprising the placental stem cells further comprise one or more additional cell types, e.g., adult cells (for example, fibroblasts or endodermal cells), or stem or progenitor cells. Such therapeutic agents and/or one or more additional cells, can be administered to an individual in need thereof individually or in combinations or two or more such compounds or agents.

4.6.2 Critical Limb Ischemia

In a specific embodiment, the disease state or condition treatable with a therapeutically effective amount of a composition comprising placental stem cells and platelet rich plasma is critical limb ischemia (CLI). Thus, in another aspect, provided herein is a method of treating an individual having CLI, comprising administering to the individual a therapeutically-effective amount of a composition comprising placental stem cells, as described herein, and platelet rich plasma.

In another more specific embodiment, said CLI is a severe blockage in the arteries of the lower extremities, which markedly reduces blood-flow. In another more specific embodiment, said CLI is characterized by ischemic rest pain, severe pain in the legs and feet while a person is not moving, non-healing sores on the feet or legs, pain or numbness in the feet, shiny, smooth, dry skin of the legs or feet, thickening of the toenails, absent or diminished pulse in the legs or feet, open sores, skin infections or ulcers that will not heal, and/or dry gangrene (dry, black skin) of the legs or feet. In another specific embodiment, CLI can lead to loss of digits and or whole limbs.

In another specific embodiment of the method, administration of said therapeutically effective amount of a composition comprising placental stem cells and platelet rich plasma results in elimination of, a detectable improvement in, lessening of the severity of, or slowing of the progression of one or more symptoms of, loss of limb function and or oxygen deprivation (hypoxia/anoxia) attributable to, a disruption of the flow of blood in or around the limb of said individual. In another specific embodiment, said therapeutically effective amount of a composition comprising placental stem cells and platelet rich plasma is administered to said individual prophylactically, e.g., to reduce or eliminate tissue damage caused by a second or subsequent disruption of flow of blood in or around the limb following said disruption of flow of blood.

In some embodiments, the CLI results from an acute condition such as an embolus or thrombosis. In some embodiments, the CLI is the end result of arterial occlusive disease, e.g., atherosclerosis. In particular embodiments, the CLI results from atherosclerosis in association with hypertension, hypercholesterolemia, cigarette smoking and diabetes. In some embodiments, the CLI results from Buerger's disease, thromboangiitis obliterans, or arteritis.

In some embodiments, the CLI is characterized by claudication, wherein narrowed vessels cannot supply sufficient blood flow to exercising leg muscles, which is brought on by exercise and relieved by rest. In some embodiments, the CLI is characterized by burning pain in the ball of the foot and toes that is worse at night when the individual is in bed. In some embodiments, the CLI is characterized by progressive gangrene, rapidly enlarging wounds and/or continuous ischemic rest pain. In some embodiments, the CLI is characterized by an ankle-brachial index of 0.4 or less, more than two weeks of recurrent foot pain at rest that requires regular use of analgesics and is associated with an ankle systolic pressure of 50 mm Hg or less, or a toe systolic pressure of 30 mm Hg or less, and/or a nonhealing wound or gangrene of the foot or toes, with similar hemodynamic measurements. Generally, a wound is considered to be nonhealing if it fails to respond to a four- to 12-week trial of conservative therapy such as regular dressing changes, avoidance of trauma, treatment of infection and debridement of necrotic tissue.

The methods for treating CLI provided herein further encompass treating CLI by administering a therapeutically effective amount of a composition comprising placental stem cells and platelet rich plasma, in conjunction with one or more therapies or treatments used in the course of treating CLI. The one or more additional therapies may be used prior to, concurrent with, or after administration of the composition comprising placental stem cells and platelet rich plasma. In some embodiments, the one or more additional therapies comprise operative intervention. In some embodiments, the operative intervention comprises surgical revascularization.

In some embodiments, the surgical revascularization comprises minimally invasive endovascular therapy. In some embodiments, the endovascular therapy comprises puncture of the groin, under local anesthesia, with insertion of a catheter into the artery in the groin which will allow access to the diseased portion of the artery, e.g., a site of plaque localization. In some embodiments, the endovascular therapy comprises angioplasty, i.e., insertion of a small balloon through a puncture in the groin, wherein the balloon is inflated one or more times, using a saline solution, to open the artery. In some embodiments, the endovascular therapy comprises insertion of a cutting balloon, i.e., a balloon imbedded with micro-blades is used to dilate the diseased area, wherein the blades cut the surface of the plaque, reducing the force necessary to dilate the vessel. In some embodiments, the endovascular therapy comprises insertion of a cold balloon, i.e., cryoplasty, wherein instead of using saline, the balloon is inflated using nitrous oxide which freezes the plaque. In some embodiments, the endovascular therapy comprises insertion of one or stents, i.e., metal mesh tubes that provide scaffolding, for example, after an artery has been opened using a balloon angioplasty. In some embodiments, the stent is a balloon-expanded stent. In some embodiments, the stent is a self-expanding stent. In some embodiments, the endovascular therapy comprises laser atherectomy, wherein small bits of plaque are vaporized by the tip of a laser probe. In some embodiments, the endovascular therapy comprises directional atherectomy, wherein a catheter with a rotating cutting blade is used to physically remove plaque from the artery, opening the flow channel.

4.6.3 Wound Healing Applications

In another specific embodiment of the methods of treatment described herein, a composition comprising placental stem cells and platelet rich plasma is used for the treatment of a wound, including but not limited to: an epidermal wound, skin wound, chronic wound, acute wound, external wound, internal wound, and a congenital wound (e.g., dystrophic epidermolysis bullosa). Thus, in another aspect, provided herein is a method of treating an individual having a wound, comprising administering to the individual a therapeutically-effective amount of a composition comprising placental stem cells, as described herein, and platelet rich plasma.

In other embodiments, a composition comprising placental stem cells and platelet rich plasma is administered to an individual for the treatment of a wound infection, e.g., a wound infection followed by a breakdown of a surgical or traumatic wound. The compositions comprising placental stem cells and platelet rich plasma described herein have therapeutic utility in the treatment of wound infections from any microorganism known in the art, e.g., microorganisms that infect wounds originating from within the human body, which is a known reservoir for pathogenic organisms, or from environmental origin. A non-limiting example of the microorganisms, the growth of which in wounds may be reduced or prevented by the methods and compositions described herein are Staphylococcus aureus, S. epidermidis, beta haemolytic streptococci, Escherichia coli, Klebsiella and Pseudomonas species, and among the anaerobic bacteria, the Clostridium welchii or C. tartium, which are the cause of gas gangrene, mainly in deep traumatic wounds.

In other embodiments, a composition comprising placental stem cells and platelet rich plasma is administered for the treatment of burns, including but not limited to, first-degree burns, second-degree burns (partial thickness burns), third degree burns (full thickness burns), infection of burn wounds, infection of excised and unexcised burn wounds, infection of grafted wound, infection of donor site, loss of epithelium from a previously grafted or healed burn wound or skin graft donor site, and burn wound impetigo.

In particular, the compositions comprising placental stem cells and platelet rich plasma described herein have enhanced utility in the treatment of ulcers, e.g., leg ulcers. In various embodiments, said leg ulcer can be, for example, a venous leg ulcer, arterial leg ulcer, diabetic leg ulcer, decubitus ulcer, or split thickness skin grafted ulcer or wound. In this context, “treatment of a leg ulcer” comprises contacting the leg ulcer with an amount of a composition comprising placental stem cells and platelet rich plasma effective to improve at least one aspect of the leg ulcer. As used herein, “aspect of the leg ulcer” includes objectively measurable parameters such as ulcer size, depth or area, degree of inflammation, ingrowth of epithelial and/or mesodermal tissue, gene expression within the ulcerated tissue that is correlated with the healing process, quality and extent of scarring etc., and subjectively measurable parameters, such as patient well-being, perception of improvement, perception of lessening of pain or discomfort associated with the ulcer, patient perception that treatment is successful, and the like.

4.6.3.1 Venous Leg Ulcers

Provided herein are methods for the treatment of venous leg ulcers comprising administering an amount of a composition comprising placental stem cells and platelet rich plasma effective to improve at least one aspect of the venous leg ulcer. Venous leg ulcers, also known as venous stasis ulcers or venous insufficiency ulcers, a type of chronic or non-healing wound, are widely prevalent in the United States, with approximately 7 million people, usually the elderly, afflicted. Worldwide, it is estimated that 1-1.3% of individuals suffer from venous leg ulcers. Approximately 70% of all leg ulcers are venous ulcers. Venous leg ulcers are often located in the distal third of the leg known as the gaiter region, and typically on the inside of the leg. The ulcer is usually painless unless infected. Venous leg ulcers typically occur because the valves connecting the superficial and deep veins fail to function properly. Failure of these valves causes blood to flow from the deep veins back out to the superficial veins. This inappropriate flow, together with the effects of gravity, causes swelling and progression to damage of lower leg tissues.

Patients with venous leg ulcers often have a history of deep vein thrombosis, leg injury, obesity, phlebitis, prior vein surgery, and lifestyles that require prolonged standing. Other factors may contribute to the chronicity of venous leg ulcers, including poor circulation, often caused by arteriosclerosis; disorders of clotting and circulation that may or may not be related to atherosclerosis; diabetes; renal (kidney) failure; hypertension (treated or untreated); lymphedema (buildup of fluid that causes swelling in the legs or feet); inflammatory diseases such as vasculitis, lupus, scleroderma or other rheumatological conditions; medical conditions such as high cholesterol, heart disease, high blood pressure, sickle cell anemia, or bowel disorders; a history of smoking (either current or past); pressure caused by lying in one position for too long; genetics (predisposition for venous disease); malignancy (tumor or cancerous mass); infections; and certain medications.

Thus, in another embodiment, provided herein is a method of treating a venous leg ulcer comprising contacting the venous leg ulcer with an amount of a composition comprising placental stem cells and platelet rich plasma sufficient to improve at least one aspect of the venous leg ulcer. In another specific embodiment, the method additionally comprises treating an underlying cause of the venous leg ulcer.

The methods for treating a venous leg ulcer provided herein further encompass treating the venous leg ulcer by administering a therapeutically effective amount of a composition comprising placental stem cells and platelet rich plasma, in conjunction with one or more therapies or treatments used in the course of treating a venous leg ulcer. The one or more additional therapies may be used prior to, concurrent with, or after administration of the composition comprising placental stem cells and platelet rich plasma. In some embodiments, the one or more additional therapies comprise compression of the leg to minimize edema or swelling. In some embodiments, compression treatments include wearing therapeutic compression stockings, multilayer compression wraps, or wrapping an ACE bandage or dressing from the toes or foot to the area below the knee.

4.6.3.2 Other Leg Ulcer Types

Arterial leg ulcers are caused by an insufficiency in one or more arteries' ability to deliver blood to the lower leg, most often due to atherosclerosis. Arterial ulcers are usually found on the feet, particularly the heels or toes, and the borders of the ulcer appear as though they have been ‘punched out’. Arterial ulcers are frequently painful. This pain is relieved when the legs are lowered with feet on the floor as gravity causes more blood to flow into the legs. Arterial ulcers are usually associated with cold white or bluish, shiny feet.

The treatment of arterial leg ulcers contrasts to the treatment of venous leg ulcers in that compression is contraindicated, as compression tends to exacerbate an already-poor blood supply, and debridement is limited, if indicated at all. Thus, in another embodiment, provided herein is a method of treating an arterial leg ulcer comprising treating the underlying cause of the arterial leg ulcer, e.g., arteriosclerosis, and contacting the arterial leg ulcer with an amount of a composition comprising placental stem cells and platelet rich plasma sufficient to improve at least one aspect of the arterial leg ulcer. In a specific embodiment, the method of treating does not comprise compression therapy.

Diabetic foot ulcers are ulcers that occur as a result of complications from diabetes. Diabetic ulcers are typically caused by the combination of small arterial blockage and nerve damage, and are most common on the foot, though they may occur in other areas affected by neuropathy and pressure. Diabetic ulcers have characteristics similar to arterial ulcers but tend to be located over pressure points such as heels, balls of the feet, tips of toes, between toes or anywhere bony prominences rub against bed sheets, socks or shoes.

Treatment of diabetic leg ulcers is generally similar to the treatment of venous leg ulcers, though generally without compression; additionally, the underlying diabetes is treated or managed. Thus, in another embodiment, provided herein is a method of treating a diabetic leg ulcer comprising treating the underlying diabetes, and contacting the diabetic leg ulcer with an amount of a composition comprising placental stem cells and platelet rich plasma sufficient to improve at least one aspect of the diabetic leg ulcer.

Decubitus ulcers, commonly called bedsores or pressure ulcers, can range from a very mild pink coloration of the skin, which disappears in a few hours after pressure is relieved on the area to a very deep wound extending into the bone. Decubitus ulcers occur frequently with patients subject to prolonged bedrest, e.g., quadriplegics and paraplegics who suffer skin loss due to the effects of localized pressure. The resulting pressure sores exhibit dermal erosion and loss of the epidermis and skin appendages. Factors known to be associated with the development of decubitus ulcers include advanced age, immobility, poor nutrition, and incontinence. Stage 1 decubitus ulcers exhibit nonblanchable erythema of intact skin. Stage 2 decubitus ulcers exhibit superficial or partial thickness skin loss. Stage 3 decubitus ulcers exhibit full thickness skin loss with subcutaneous damage. The ulcer extends down to underlying fascia, and presents as a deep crater. Finally, stage 4 decubitus ulcers exhibit full thickness skin loss with extensive destruction, tissue necrosis, and damage to the underlying muscle, bone, tendon or joint capsule. Thus, in another embodiment, provided herein is a method of treating a decubitus leg ulcer comprising treating the underlying diabetes, and contacting the decubitus leg ulcer with an amount of a composition comprising placental stem cells and platelet rich plasma sufficient to improve at least one aspect of the decubitus leg ulcer.

Also provided herein are methods of treating a leg ulcer by administering a composition comprising placental stem cells and platelet rich plasma in conjunction with one or more therapies or treatments used in the course of treating a leg ulcer. The one or more additional therapies may be used prior to, concurrent with, or after administration of the composition comprising placental stem cells and platelet rich plasma. A composition comprising placental stem cells and platelet rich plasma, and one or more additional therapies, may be used where the composition comprising placental stem cells and platelet rich plasma, alone, or the one or more additional therapies, alone, would be insufficient to measurably improve, maintain, or lessen the worsening of, one or more aspects of a leg ulcer. In specific embodiments, the one or more additional therapies comprise, without limitation, treatment of the leg ulcer with a wound healing agent (e.g., PDGF, REGRANEX®); administration of an anti-inflammatory compound; administration of a pain medication; administration of an antibiotic; administration of an anti-platelet or anti-clotting medication; application of a prosthetic; application of a dressing (e.g., moist to moist dressings; hydrogels/hydrocolloids; alginate dressings; collagen-based wound dressings; antimicrobial dressings; composite dressings; synthetic skin substitutes, etc.), and the like. In another embodiment, the additional therapy comprises contacting the leg ulcer with honey. For any of the above embodiments, in a specific embodiment, the leg ulcer is a venous leg ulcer, a decubitus ulcer, a diabetic ulcer, or an arterial leg ulcer.

In another specific embodiment, the additional therapy is a pain medication. Thus, also provided herein is a method of treating a leg ulcer comprising contacting the leg ulcer with a composition comprising placental stem cells and platelet rich plasma, and administering a pain medication to lessen or eliminate leg ulcer pain. In a specific embodiment, the pain medication is a topical pain medication.

In another specific embodiment, the additional therapy is an anti-infective agent. Preferably, the anti-infective agent is one that is not cytotoxic to healthy tissues surrounding and underlying the leg ulcer; thus, compounds such as iodine and bleach are disfavored. Thus, treatment of the leg ulcer, in one embodiment, comprises contacting the leg ulcer with a composition comprising placental stem cells and platelet rich plasma, and administering an anti-infective agent. The anti-infective agent may be administered by any route, e.g., topically, orally, buccally, intravenously, intramuscularly, anally, etc. In a specific example, the anti-infective agent is an antibiotic, a bacteriostatic agent, antiviral compound, a virustatic agent, antifungal compound, a fungistatic agent, or an antimicrobial compound. In another specific embodiment, the anti-infective agent is ionic silver. In a more specific embodiment, the ionic silver is contained within a hydrogel. In specific embodiments, the leg ulcer is a venous leg ulcer, arterial leg ulcer, decubitus ulcer, or diabetic ulcer.

4.6.4 Orthopedic Applications

In another specific embodiment of the methods of treatment described herein, a composition comprising placental stem cells and platelet rich plasma is used for the treatment of orthopedic defects, including but not limited to, bone defects, disc herniation and degenerative disc disease. Thus, in another aspect, provided herein is a method of treating an individual having a bone defect, disc herniation, or degenerative disc disease, comprising administering to the individual a therapeutically-effective amount of a composition comprising placental stem cells, as described herein, and platelet rich plasma.

In a particular aspect, provided herein is a method for treating a bone defect in a subject, comprising administering to a subject in need thereof a therapeutically effective amount of an implantable or injectable composition comprising placental stem cells and platelet rich plasma sufficient to treat the bone defect in the subject. In certain embodiments, the bone defect is an osteolytic lesion associated with a cancer, a bone fracture, or a spine, e.g., in need of fusion. In certain embodiments, the osteolytic lesion is associated with multiple myeloma, bone cancer, or metastatic cancer. In certain embodiments, the bone fracture is a non-union fracture. In certain embodiments, an implantable composition comprising placental stem cells and platelet rich plasma is administered to the subject. In certain embodiments, an implantable composition is surgically implanted, e.g., at the site of the bone defect. In certain embodiments, an injectable composition comprising placental stem cells and platelet rich plasma is administered to the subject. In certain embodiments, an injectable composition is surgically administered to the region of the bone defect.

4.6.4.1 Disc Herniation and Degenerative Disc Disease

In particular, the compositions comprising placental stem cells and platelet rich plasma described herein have enhanced utility in the treatment of herniated discs and degenerative disc disease. In some embodiments, the degenerative disc disease is characterized on x-ray tests or MRI scanning of the spine as a narrowing of the normal “disc space” between the adjacent vertebrae.

Disc degeneration, medically referred to as spondylosis, can occur with age when the water and protein content of the cartilage of the body changes. This change results in weaker, more fragile and thin cartilage. Because both the discs and the joints that stack the vertebrae (facet joints) are partly composed of cartilage, these areas are subject to degenerative changes, which renders the disc tissue susceptible to herniation. The gradual deterioration of the disc between the vertebrae is referred to as degenerative disc disease. Degeneration of the disc can cause local pain in the affected area, for example, radiculopathy, i.e., nerve irritation caused by damage to the disc between the vertebrae. In particular, weakness of the outer ring leads to disc bulging and herniation. As a result, the central softer portion of the disc can rupture through the outer ring of the disc and abut the spinal cord or its nerves as they exit the bony spinal column.

Any level of the spine can be affected by disc degeneration. Thus, in some embodiments, the degenerative disc disease treatable by the methods provided herein is cervical disc disease, i.e., disc degeneration that affects the spine of the neck, often accompanied by painful burning or tingling sensations in the arms. In some embodiments, the degenerative disc disease is thoracic disc disease, i.e., disc degeneration that affects the mid-back. In some embodiments, the degenerative disc disease is lumbago, i.e., disc degeneration that affects the lumbar spine.

In particular embodiments, the method for treating degenerative disc disease in a subject comprises administering to a subject in need thereof a therapeutically effective amount of an implantable or injectable composition comprising placental stem cells and platelet rich plasma sufficient to treat cervical or lumbar radiculopathy in the subject. In some embodiments, the lumbar radiculopathy is accompanied by incontinence of the bladder and/or bowels. In some embodiments, the method for treating degenerative disc disease in a subject comprises administering to a subject in need thereof a therapeutically effective amount of an implantable or injectable composition comprising placental stem cells and platelet rich plasma sufficient to relieve sciatic pain in the subject.

In some embodiments of the methods of treating disc degeneration in an individual with a composition comprising placental stem cells and platelet rich plasma, as provided herein, disc degeneration of the individual occurs at the intervertebral disc between C1 and C2; between C2 and C3; between C3 and C4; between C4 and C5; between C5 and C6; between C6 and C7; between C7 and T1; between T1 and T2; between T2 and T3; between T3 and T4; between T4 and T5; between T5 and T6; between T6 and T7; between T7 and T8; between T8 and T9; between T9 and T10; between T10 and T11; between T11 and T12; between T12 and L1; between L1 and L2; between L2 and L3; between L3 and L4; or between L4 and L5.

In some embodiments of the methods of treating disc herniation in an individual with a composition comprising placental stem cells and platelet rich plasma, as provided herein, the disc herniation occurs at the intervertebral disc between C1 and C2; between C2 and C3; Between C3 and C4; between C4 and C5; between C5 and C6; between C6 and C7; between C7 and T1; between T1 and T2; between T2 and T3; between T3 and T4; between T4 and T5; between T5 and T6; between T6 and T7; between T7 and T8; between T8 and T9; between T9 and T10; between T10 and T11; between T11 and T12; between T12 and L1; between L1 and L2; between L2 and L3; between L3 and L4; or between L4 and L5.

Degenerative arthritis (osteoarthritis) of the facet joints is also a cause of localized lumbar pain that can be detected with plain x-ray testing. Wear of the facet cartilage and the bony changes of the adjacent joint is referred to as degenerative facet joint disease or osteoarthritis of the spine.

The methods for treating degerative disc disease provided herein further encompass treating degerative disc disease by administering a therapeutically effective amount of a composition comprising placental stem cells and platelet rich plasma, in conjunction with one or more therapies or treatments used in the course of treating degerative disc disease. The one or more additional therapies may be used prior to, concurrent with, or after administration of the composition comprising placental stem cells and platelet rich plasma. In some embodiments, the one or more additional therapies comprise administration of medications to relieve pain and muscles spasm, cortisone injection around the spinal cord (epidural injection), physical therapy (heat, massage, ultrasound, electrical stimulation), and rest (not strict bed rest, but avoiding re-injury).

In some embodiments, the one or more additional therapies comprise operative intervention, for example, where the subject presents with unrelenting pain, severe impairment of function, or incontinence (which can indicate spinal cord irritation). In some embodiments, the operative intervention comprises removal of the herniated disc with laminotomy (producing a small hole in the bone of the spine surrounding the spinal cord), laminectomy (removal of the bony wall adjacent to the nerve tissues), by needle technique through the skin (percutaneous discectomy), disc-dissolving procedures (chemonucleolysis), and others.

4.6.5 Treatment of Chronic Pain

In another specific embodiment of the methods of treatment described herein, a composition comprising placental stem cells and platelet rich plasma is used for the treatment of chronic pain. Chronic pain, e.g., neuropathic pain, a condition that afflicts at least 30% of Americans, is caused, e.g., by disorders of the nervous system, also known as neuropathy, and can be accompanied by, or caused by, tissue damage, including nerve fibers that are damaged, dysfunction or injured. Neuropathic pain may also be caused by, e.g., pathologic lesions, neurodegeneration processes, or prolonged dysfunction of parts of the peripheral or central nervous system. However, neuropathic pain can also be present when no discernible tissue damage is evident.

Neuropathic pain is generally regarded as having two components: central plasticity, e.g., as a result of changes in receptor population or receptor sensitivity at any level of the CNS, and changes in peripheral nerves, neurons and microglial, which are mediators of central sensitization of the spinal cord. Such sensitization is known to play a major role in mediating chronic inflammatory pain and neuropathic pain.

Thus, in another aspect, provided herein is a method of treating an individual having chronic pain comprising administering to the individual a therapeutically-effective amount of a composition comprising placental stem cells, as described herein, and platelet rich plasma. In a specific embodiment, the chronic pain, e.g., neuropathic pain, is, or is caused by, neuritis (e.g., polyneuritis, brachial neuritis, optic neuritis, vestibular neuritis, cranial neuritis, or arsenic neuritis), diabetes mellitus (e.g., diabetic neuropathy), peripheral neuropathy, reflex sympathetic dystrophy syndrome, phantom limb pain, post-amputation pain, postherpetic neuralgia, shingles, central pain syndrome (pain caused, e.g., by damage to the brain, spinal cord and/or brainstem), Guillain-Barre Syndrome, degenerative disc disease, cancer, multiple sclerosis, kidney disorders, alcoholism, human immunodeficiency virus-related neuropathy, Wartenberg's Migratory Sensory Neuropathy, fibromyalgia syndrome, causalgia, spinal cord injury, or exposure to a chemical agent, e.g., trichloroethylene or dapsone (diaminyl-diphenyl sulfone). In specific embodiments, the peripheral neuropathy is mononeuropathy (damage to a single peripheral nerve); polyneuropathy (damage to more than one peripheral nerve, frequently sited in different parts of the body), mononeuritis multiplex (simultaneous or sequential damage to noncontiguous nerve trunks), or autonomic neuropathy. Peripheral neuropathy, e.g., mononeuritis multiplex, may be caused by, e.g., diabetes mellitus, vasculitis (e.g., polyarteritis nodosa, Wegener granulomatosis, or Churg-Strauss syndrome), rheumatoid arthritis, lupus erythematosus (SLE), sarcoidosis, an amyloidosis, or cryoglobulinemia.

As used herein, “therapeutically effective amount” is an amount of the composition sufficient to result in a detectable, or reportable, lessening of said chronic pain. The lessening of pain may be, e.g., self-reported by the individual, or may be determined by physiological signs responsive to pain, e.g. elevated blood pressure, anxiety, and the like. Levels of neuropathic pain may be assessed, e.g., by the Visual Analog Scale (VAS), Numeric Pain Intensity Scale, Graphic Rating Scale, Verbal Rating Scale, Pain Faces Scale (Faces Pain Scale), Numeric Pain Intensity & Pain Distress Scales, Brief Pain Inventory (BPI), Memorial Pain Assessment, Alder Hey Triage Pain Score, Dallas Pain Questionnaire, Dolorimeter Pain Index (DPI), Face Legs Activity Cry Consolability Scale, Lequesne Scale, McGill Pain Questionnaire (MPQ), Descriptor differential scale (DDS), Neck Pain and disability Scale (NPAD), Numerical 11-Point Box (BS-11), Roland-Morris Back Pain Questionnaire, or the Wong-Baker FACES Pain Rating Scale. An improvement after administration of the composition to the individual in one or more of these assessments of pain is considered therapeutically effective.

In a specific embodiment, the composition comprising placental stem cells and PRP is administered to said individual locally, e.g., at one or more sites of, or adjacent to, nerve damage that causes said chronic pain, e.g., neuropathic pain. In certain specific embodiments, the composition is administered epicutaneously, subsutaneously, intradermally, subdermally, intramuscularly, intranasally, intrathecally, intraperitoneally, intraosseously, intravesically, epidurally, intracerebrally, intracerebroventricularly, or the like. In certain specific embodiments, the composition is administered locally within 0.5, 1.0, 2.0, 3.0, 4.0 or 5.0 from the site of an injury that causes or is associated with neuropathic pain, or from the site of nerve injury that causes or is associated with neuropathic pain. In certain other specific embodiments, the composition is administered locally within 0.5, 1.0, 2.0, 3.0, 4.0 or 5.0 from the site of perceived pain, e.g., that area or areas on the individual's body in which the individual perceived the neuropathic pain.

The composition can be, for example, administered locally, distally from a site of neuropathic pain, to a nerve or set of nerves that serve a damaged area of the body of an individual, e.g., an area of the body in which the individual is experiencing the neuropathic pain. For example, the composition can be administered along the spine at any point at which nerve trunks emerge from the spinal column, e.g., any of the cervical nerves, thoracic nerves, or lumbar nerves. In specific embodiments, the composition can be administered adjacent to the spinal cord at which point nerves emerging at C1, C2, C3, C4, C5, C6, or C7, or T1, T2, T3, T4, T5, T6, T7, T8, T9, T10, T11 or T12, or L1, L2, L3, L4 or L5, or at the sacrum.

4.6.6 Immune Disorders

In another specific embodiment of the methods of treatment described herein, a composition comprising placental stem cells and platelet rich plasma is used for the treatment of an immune disorder. In particular, provided herein is a method of treating an individual having or at risk of developing a disease or disorder associated with or caused by an inappropriate or unwanted immune response, comprising administering to the individual a therapeutically effective amount of a composition comprising placental stem cells and platelet rich plasma, wherein said therapeutically effective amount is an amount sufficient to cause a detectable improvement in one or more symptoms of said disease, disorder or condition.

In some embodiments, the disease or disorder is an allergy, asthma, or a reaction to an antigen exogenous to said individual. In some embodiments, the disease or disorder is graft-versus-host disease. In some embodiments, the disease or disorder is an autoimmune disease that can be treated locally. In some embodiments, the autoimmune disease is inflammatory bowel disease, rheumatoid arthritis, psoriasis, lupus erythematosus, diabetes, mycosis fungoides, or scleroderma.

In certain embodiments, the autoimmune disease is one or more of Addison's disease, alopecia greata, ankylosing spondylitis, antiphospholipid antibody syndrome, autoimmune gastritis, autoimmune hemolytic anemia, autoimmune hepatitis, autoimmune inner ear disease, autoimmune thrombocytopenic purpura, Balo disease, Behcet's disease, bullous pemphigoid, cardiomyopathy, celiac disease, chronic inflammatory demyelinating polyneuropathy, cicatrical pemphigoid (e.g., mucous membrane pemphigoid), cold agglutinin disease, degos disease, dermatitis hepatiformis, dermatomyositis (juvenile), essential mixed cryoglobulinemia, Goodpasture's syndrome, Graves' disease, Guillain-Barre syndrome, Hashimoto's thyroiditis (Hashimoto's disease; autoimmune thyroditis), idiopathic pulmonary fibrosis, idiopathic thrombocytopenia purpura, IgA nephropathy, juvenile arthritis, lichen planus, Ménière disease, mixed connective tissue disease, morephea, neuromyotonia, pemphigus vulgaris, pernicious anemia, polyarteritis nodosa, polychondritis, polymyalgia rheumatica, polymyositis (e.g., with dermatomyositis), primary agammaglobulinemia, primary biliary cirrhosis, Raynaud disease (Raynaud phenomenon), Reiter's syndrome, relapsing polychondritis, rheumatic fever, Sjogren's syndrome, stiff-person syndrome (Moersch-Woltmann syndrome), Takayasu's arteritis, temporal arteritis (giant cell arteritis), uveitis, vasculitis (e.g., vasculitis not associated with lupus erythematosus), vitiligo, and/or Wegener's granulomatosis.

In some embodiments, the inflammatory bowel disease is Crohn's disease. In some embodiments, the Crohn's disease is gastroduodenal Crohn's disease, jejunoileitis, ileocolitis, or Crohn's colitis. In some embodiments, the inflammatory bowel disease is ulcerative colitis. In some embodiments, the ulcerative colitis is pancolitis, limited colitis, distal colitis, or proctitis. In some embodiments, the symptom is one or more of inflammation and swelling of a part of the GI tract, abdominal pain, frequent emptying of the bowel, diarrhea, rectal bleeding, anemia, weight loss, arthritis, skin problems, fever, thickening of the intestinal wall, formation of scar tissue in the intestine of the individual, formation of sores or ulcers in the intestine of the individual, development of one or more fistulas in the wall of the intestinal of the individual, development of one or more fissures in the anus of the individual, development of nutritional deficiencies (e.g., deficiencies in one or more of proteins, calories, vitamins), development of kidney stones, or development of gallstones. In some embodiments, the symptom is one or more of abdominal pain, bloody diarrhea, fevers, nausea, abdominal cramps, anemia, fatigue, weight loss, loss of appetite, rectal bleeding, loss of bodily fluids and nutrients, skin lesions, joint pain, growth failure, osteoporosis, eye inflammation, or liver disease.

In some embodiments, the disease or disorder is scleroderma. In some embodiments, the scleroderma is diffuse scleroderma, limited scleroderma (CREST syndrome), morphea, or linear scleroderma. In some embodiments, the symptoms comprise one or more of hardening of the skin of the face, hardening of the skin of the fingers, Reynaud's syndrome, inappropriate vasoconstriction in an extremity, calcinosis, telangiectasia, or esophageal dysmotility.

In some embodiments, the disease or disorder is psoriasis. In some embodiments, the symptom of psoriasis is psoriatic arthritis. In some embodiments, the symptom of psoriasis is one or more of scaling of the skin, redness of the skin, thickening of the skin, formation of plaques, discoloration under the nail plate, pitting of the nails, lines going across the nails, thickening of the skin under the nail, onycholysis, development of pustules, joint or connective tissue inflammation, inflammation of the skin, or exfoliation of the skin.

In some embodiments, the disease or disorder is rheumatoid arthritis. In some embodiments, the rheumatoid arthritis involves one or more of pyoderma gangrenosum, neutrophilic dermatosis, Sweet's syndrome, viral infection, erythema nodosum, lobular panniculitis, atrophy of digital skin, palmar erythema, diffuse thinning (rice paper skin), skin fragility, subcutaneous nodules on an exterior surface, e.g., on the elbows, fibrosis of the lungs (e.g., as a consequence of methotrexate therapy), Caplan's nodules, vasculitic disorders, nail fold infarcts, neuropathy, nephropathy, amyloidosis, muscular pseudohypertrophy, endoscarditis, left ventricular failure, valulitis, scleromalacia, mononeuritis multiplex, and atlanto-axial subluxation.

In some embodiments, the disease or disorder is lupus erythematosus. In some embodiments, the symptom of lupus erythematosus is one or more of malar rash, development of thick red scaly patches on the skin, alopecia, mouth ulcers, nasal ulcers, vaginal ulcers, skin lesions, joint pain, anemia deficiency, iron deficiency, lower than normal platelet and white blood cell counts, antiphospholipid antibody syndrome, presence of anticardiolipin antibody in the blood, pericarditis, myocarditis, endocarditis, lung inflammation, pleural inflammation, pleuritis, pleural effusion, lupus pneumonitis, pulmonary hypertension, pulmonary emboli, pulmonary hemorrhage, autoimmune hepatitis, jaundice, presence of antinuclear antibody (ANA) in the blood, presence of smooth muscle antibody (SMA) in the blood, presence of liver/kidney microsomal antibody (LKM-1) in the blood, presence of anti-mitochondrial antibody (AMA) in the blood, hematuria, proteinuria, lupus nephritis, renal failure, development of membranous glomerulonephritis with “wire loop” abnormalities, seizures, psychosis, abnormalities in the cerebrospinal fluid, deficiency in CD45 phosphatase and/or increased expression of CD40 ligand in T cells of the individual, lupus gastroenteritis, lupus pancreatitis, lupus cystitis, autoimmune inner ear disease, parasympathetic dysfunction, retinal vasculitis, systemic vasculitis, increased expression of FcεRIγ, increased and sustained calcium levels in T cells, increase of inositol triphosphate in the blood, reduction in protein kinase C phosphorylation, reduction in Ras-MAP kinase signaling, or a deficiency in protein kinase A I activity.

In some embodiments, the disease, disorder or condition is mycosis fungoides (Alibert-Bazin syndrome). In some embodiments, the mycosis fungoides is in the patch phase. In some embodiments, the mycosis fungoides is in the skin tumor phase. In some embodiments, the mycosis fungoides is in the skin redness (erythroderma) stage. In some embodiments, the mycosis fungoides is in the lymph node stage. In some embodiments, the symptom is one or more of development of flat red patches that are itchy, development of flat, red patches that are raised and hard (plaques), development of raised lumps (nodules), development of large red itchy scaly areas over the body, cracking of the skin of the palms and soles, thickening of the skin of the palms and soles, or inflammation of the lymph nodes.

### 5. EXAMPLES

### 5.1 Example 1

### In Vivo Model for Treating Critical Limb Ischemia with Compositions Comprising Placental Stem Cells and PRP

This example describes experiments that are performed in order to assess treatment of critical limb ischemia with compositions comprising CD10⁺, CD34⁻, CD105⁺, CD200⁺ placental stem cells, also called PDACs, and platelet rich plasma (PRP).

In brief, two rodent hind limb ischemia models are surgically induced, as described by Goto et al., Tokai J. Exp. Clin. Med. 31(3):128-132 (2006). These include: (1) a chronic mild ischemia model, which is induced by cutting the femoral artery just below the bifurcation of the deep femoral artery; and (2) a stable severe ischemia model, which is induced by resection of the femoral artery from the distal site of the bifurcation of the deep femoral artery to the saphenous artery. Each group is subsequently treated with PDACs only, PRP only, and PDACs in combination with PRP. The amounts of PDACs, and the ratio of PDACs to PRP, are varied to assess dose-dependency of the different treatments.

Blood flow, in particular, calf blood flows on both sides are measured below a patella with a noncontact laser Doppler flowmeter before the surgical induction of ischemia, just after the surgical induction, before administration of the compositions as described above, and two weeks post-administration, and are expressed as the ratio of the flow in the ischemic limb to that in the normal limb, for each treatment group. At two weeks post-administration, the animals are sacrificed under an overdose of sodium pentobarbital and the anterotibial, gastrocnemius, and soleus muscles are dissected out and weighed. Histological analysis (HE staining) is performed in each muscle.

### 5.2 Example 2

### In Vivo Models for Treating Bone Repair and Disc Degeneration with Compositions Comprising Placental Stem Cells and PRP

This example describes experiments that are performed in order to assess treatment of bone defects with compositions comprising CD10⁺, CD34⁻, CD105⁺, CD200⁺ placental stem cells, also called PDACs, and platelet rich plasma (PRP). Several models of bone disease are adapted to assess the efficacy of such treatments on different bone diseases.

To model cranial bilateral defect, a defect of 3 mm×5 mm is surgically created on each side of the cranium of male athymic rats. The defects are treated with matrix only, PDACs only, PRP only, matrix in combination with PDACs, matrix in combination with PRP, and matrix in combination with PDACs and PRP. The amounts of PDACs, and the ratio of PDACs to PRP, are varied to assess dose-dependency of the different treatments. Different matrix materials are also assessed in order to test the effects of different combinations of matrix, stem cells, and PRP.

Six rats are assigned to each treatment group and the defects are filled with the designated matrix and cell combination. At four weeks, serum is collected and rats are sacrificed. Serum is tested for immunologic reaction to the implants. Rat crania are collected for microradiography and placed in 10% NBF.

Calvariae are processed for paraffin embedding and sectioning. Coronal histological sections of the calvariae are stained with toluidine stain according to conventional techniques. Bone ingrowth into the defect and remnant of matrix carrier is assessed according to a 0 to 4 scale, with four being the largest amount of ingrowth. Inflammation and fibrosis is also assessed.

Treatment of bone lesions resulting from cancer metastases can be assessed according to an adaptation of the procedure of Bäuerle et al., 2005, Int. J. Cancer 115:177-186. Briefly, site-specific osteolytic lesions are induced in nude rats by intra-arterial injection of human breast cancer cells into an anastomosing vessel between the femoral and the iliac arteries. The metastases are then either treated with conventional anti-cancer therapies (e.g., chemotherapeutic, radiological, immunological, or other therapy) or surgically removed. Next, the lesions remaining from the cancer metastases are filled with different matrix combinations as described above. After an appropriate period of time, as determined by radiologically monitoring the animals, the animals are sacrificed. Immunologic response against the matrix, inflammation, fibrosis, degree of bone ingrowth, and amount of matrix carrier are assessed.

Additional references that describe models of bone disease that can be used or adapted to assess the efficacy of compositions comprising placental stem cells and platelet rich plasma to treat bone defects include Mitsiades et al., 2003, Cancer Res. 63:6689-96; Chakkalakal et al., 2002, Alcohol Alcoholism 37:13-20; Chiba et al., 2001, J. Vet. Med. Sci. 63:603-8; Garrett et al., 1997, Bone 20:515-520; and Miyakawa et al., 2003, Biochem. Biophys. Res. Comm. 313:258-62.

Treatment of disc degeneration can be assessed according to an adaptation of the procedure of Olmarker et al., 2008, Spine 33(8):850-855. In brief, rats are subjected to sham exposure or disc puncture. In rats receiving sham exposure only, the left facet joint between the 4th and the 5th lumbar vertebra is removed and the 4^{th} lumbar dorsal root ganglion and the 5th lumbar nerve root, including the intervertebral disc between the fourth and fifth lumbar vertebrae (L4 and L5, respectively), are visualized. In rats subjected to disc puncture, the L4-L5 intervertebral disc is further punctured using a 0.4-mm diameter injection needle. Leakage of the nucleus pulposus is facilitated by injecting a small amount of air into the center of the disc.

Rats subjected to sham exposure or punctured discs are treated with PDACs only, PRP only, and PDACs in combination with PRP. The amounts of PDACs, and the ratio of PDACs to PRP, are varied to assess dose-dependency of the different treatments. Six rats are assigned to each treatment group and the defects are filled with the designated matrix and cell combination. The spinal muscles are sutured and the skin is closed by metal-clips.

After surgery, each rat receives a unique identification number to allow for a blinded behavioral assessment. Behavioral Testing Behavioral analysis is performed on days 1, 3, 7, 14, and 21 after surgery. Olmarker et al. reported that rats subjected to disc puncture, when compared to rats receiving only sham exposure, display increased grooming behavior and “wet-dog shaking” (WDS), a behavior that resembles a wet dog that is shaking to remove water from the fur. These two behaviors are suggested to indicate stress and pain. Thus, the ability of PDACs and PRP, alone or in combination, to suppress or ameliorate these behaviors in rats subjected to disc puncture are assessed.

### 5.3 Example 3

### In Vivo Model for Treating Neuropathic Pain with Compositions Comprising Placental Stem Cells and PRP

This Example provides an exemplary model and method for evaluating the effects of a composition comprising PDACs and PRP in a rat model for chronic, painful peripheral mononeuropathy.

Peripheral mononeuropathy is surgically induced in rats as described by Bennett et al., Pain 33:87-107 (1988). In brief, rats are anesthetized with sodium pentobarbital (40 mg/kg. i.p.). The common sciatic nerve is exposed at the level of the middle of the thigh by blunt dissection through biceps femoris. Proximal to the sciatic's trifurcation, about 7 mm of nerve is freed of adhering tissue and 4 ligatures (4.0 chromic gut) are tied loosely around it with about 1 mm spacing. The length of nerve thus affected is 4 5 mm long. The ligatures are tied such that the diameter of the nerve is seen to be just barely constricted when viewed with 40× magnification. The desired degree of constriction retards, but does not arrest, circulation through the superficial epineurial vasculature. The incision is closed in layers. In each animal, an identical dissection is performed on the opposite side, except that the sciatic nerve is not ligated. Groups of control rats are used, wherein some rats are not operated upon and others receive bilateral sham procedures (sciatic exposure without ligation).

Each group is subsequently treated with PDACs only, PRP only, and PDACs in combination with PRP. The amounts of PDACs, and the ratio of PDACs to PRP, are varied to assess dose-dependency of the different treatments. The animals are inspected every 1 or 2 days during the first 14 postoperative days and at about weekly intervals thereafter. During these inspections, each rat is placed upon a table and carefully observed for 1-2 minutes. Notes are made of the animal's gait, the posture of the affected hind paw, the condition of the hind paw skin, and the extent, if present, of autotomy. Particular attention is given to the condition of the claws because autotomy involving frank tissue damage can be indicated by gnawed claw tips. Postoperative, post-administration behavior of the rats is observed, including appetite and hyperalgesic responses to noxious radiant heat and chemogenic pain.

Assessment of Response to Noxious Heat

The rats are placed beneath an inverted, clear plastic cage (18×28×13 cm) upon an elevated floor of window glass. A radiant heat source beneath the glass floor is aimed at the plantar hind paw. Stimulus onset activates a timer controlled by a photocell positioned to receive light reflected from the hind paw. The hind paw withdrawal reflex interrupts the photocell's light and automatically stopped the timer. Latencies are measured to the nearest 0.1 sec. The hind paws are tested alternately with 5 min intervals between consecutive tests. Five latency measurements are taken for each hind paw in each test session. The 5 latencies per side are averaged and a difference score is computed by subtracting the average latency of the control side from the average latency of the ligated side. Difference scores are compared for each treatment group, i.e., PDACs only, PRP only, and PDACs combined with PRP.

Assessment of Response to Noxious Pressure Stimulation

A conical stylus with a hemispherical tip (1.2 mm radius) is placed upon the middle of hind paw dorsum between the second and third or third and fourth metatarsals. The animal is restrained gently between cupped hands and calibrated pressure of gradually increasing (ca. 25.5 g/sec) intensity is applied until the rat withdraws the hind paw. The hind paws are tested alternately at 3-4 min intervals. Three measurements are taken for each side, averaged, and a difference score computed by subtracting the average of the control side from the average of the ligated side. Difference scores are compared for each treatment group, i.e., PDACs only, PRP only, and PDACs combined with PRP.

### EQUIVALENTS

The compositions and methods disclosed herein are not to be limited in scope by the specific embodiments described herein. Indeed, various modifications of the compositions and methods in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

1. A composition comprising placental stem cells and platelet rich plasma, wherein said composition is suitable for injection into an individual, and wherein said placental stem cells are adherent to tissue culture plastic, are CD34⁻, CD10⁺, CD105⁺ and CD200⁺, and are not trophoblasts.

2. (canceled)

3. (canceled)

4. The composition of claim 1, wherein injection of said composition to said individual results in prolonged localization of said placental stem cells at the site of injection, relative to injection of placental stem cells not combined with platelet rich plasma.

5. The composition of claim 1, wherein said placental stem cells express CD200 and do not express HLA-G, or express CD73, CD105, and CD200, or express CD200 and OCT-4, or express CD73 and CD105 and do not express HLA-G, or express CD73 and CD105 and facilitate the formation of one or more embryoid-like bodies in a population of placental cells comprising said stem cell when said population is cultured under conditions that allow for the formation of an embryoid-like body, or express OCT-4 and facilitate the formation of one or more embryoid-like bodies in a population of placental cells comprising said stem cell when said population is cultured under conditions that allow for the formation of an embryoid-like body.

6. The composition of claim 1, wherein said platelet rich plasma is autologous platelet rich plasma.

7. The composition of claim 1, wherein said platelet rich plasma is derived from placental perfusate.

8. (canceled)

9. (canceled)

10. The composition of claim 1, wherein the ratio of the number of placental stem cells to the number of platelets in the platelet rich plasma is between about 100:1 and 1:100.

11. (canceled)

12. A method of transplantation comprising administering the composition of claim 1 by injection, wherein said injection results in prolonged localization of said placental stem cells at the site of injection, as compared to injection of placental stem cells not combined with platelet rich plasma, wherein said placental stem cells are adherent to tissue culture plastic, are CD34⁻, CD10⁺, CD105⁺ and CD200⁺, and are not trophoblasts.

13. The method of claim 12, wherein said placental stem cells are not obtained from umbilical cord.

14. The method of claim 12, wherein said composition does not comprise an implantable bone substitute, and does not require thrombin to retain said placental stem cells at a site of said injection of said composition into said individual.

15. The method of claim 12, wherein said placental stem cells express CD200 and do not express HLA-G, or express CD73, CD105, and CD200, or express CD200 and OCT-4, or express CD73 and CD105 and do not express HLA-G, or express CD73 and CD105 and facilitate the formation of one or more embryoid-like bodies in a population of placental cells comprising said stem cell when said population is cultured under conditions that allow for the formation of an embryoid-like body, or express OCT-4 and facilitate the formation of one or more embryoid-like bodies in a population of placental cells comprising said stem cell when said population is cultured under conditions that allow for the formation of an embryoid-like body.

16. The method of claim 12, wherein said platelet rich plasma is autologous platelet rich plasma.

17. The method of claim 12, wherein said platelet rich plasma is derived from placental perfusate.

18. The method of claim 12, wherein said placental stem cells and said platelet rich plasma are combined to form said composition ex vivo prior to said injecting the individual.

19. The method of claim 12, wherein said platelet rich plasma is injected into the individual in a first step, and said placental stem cells are injected into or near the site of platelet rich plasma injection in a second step, and said composition is formed in vivo.

20. The method of claim 12, wherein transplantation of said composition comprising placental stem cells and platelet rich plasma prolongs localization of the placental stem cells at the site of injection or implantation for 1, 2, 3, 4, 5, 6, 7, 8, 9, 10, 11, 12, 13, 14, 15, 16, 17, 18, 19, 20 or 21 days or more, post-transplant, relative to transplantation of placental stem cells not combined with platelet rich plasma.

21. (canceled)

22. (canceled)

23. The method of claim 12, wherein the ratio of the number of placental stem cells to the number of platelets in the platelet rich plasma is between about 100:1 and 1:100.

24. (canceled)

25. A method of treating an individual having or critical limb ischemia, comprising administering to the individual a therapeutically effective amount of a composition comprising placental stem cells and platelet rich plasma, wherein said placental stem cells are adherent to tissue culture plastic, are CD34⁻, CD10⁺, CD105⁺ and CD200⁺, and are not trophoblasts.

26. (canceled)

27. (canceled)

28. (canceled)

29. (canceled)

30. (canceled)

31. A method of treating an individual having or at risk of developing a disease or disorder associated with or caused by an inappropriate or unwanted immune response, comprising administering to the individual a therapeutically effective amount of a composition comprising placental stem cells and platelet rich plasma, wherein said therapeutically effective amount is an amount sufficient to cause a detectable improvement in one or more symptoms of said disease, disorder or condition, and wherein said placental stem cells are adherent to tissue culture plastic, are CD34⁻, CD10⁺, CD105⁺ and CD200⁺, and are not trophoblasts

32. (canceled)

33. The method of claim 31, wherein said composition does not comprise an implantable bone substitute, and does not require thrombin to retain said placental stem cells at a site of said injection of said composition into said individual.

34. The method of claim 31, wherein said placental stem cells express CD200 and do not express HLA-G, or express CD73, CD105, and CD200, or express CD200 and OCT-4, or express CD73 and CD105 and do not express HLA-G, or express CD73 and CD105 and facilitate the formation of one or more embryoid-like bodies in a population of placental cells comprising said stem cell when said population is cultured under conditions that allow for the formation of an embryoid-like body, or express OCT-4 and facilitate the formation of one or more embryoid-like bodies in a population of placental cells comprising said stem cell when said population is cultured under conditions that allow for the formation of an embryoid-like body.

35. (canceled)

36. (canceled)

37. (canceled)

38. (canceled)

39. (canceled)

40. The method of claim 31, wherein transplantation of said composition comprising placental stem cells and platelet rich plasma prolongs localization of the placental stem cells at the site of injection or implantation for 1, 2, 3, 4, 5, 6, 7, 8, 9, 10, 11, 12, 13, 14, 15, 16, 17, 18, 19, 20 or 21 days or more, post-transplant, relative to transplantation of placental stem cells not combined with platelet rich plasma.

41. (canceled)

42. (canceled)

43. (canceled)

44. (canceled)

45. (canceled)

46. (canceled)

47. The method of claim 31, wherein said disease or disorder is an allergy, asthma, or a reaction to an antigen exogenous to said individual.

48. The method of claim 31, wherein said disease or disorder is graft-versus-host disease.

49. (canceled)

50. The method of claim 49, wherein said autoimmune disease is multiple sclerosis, inflammatory bowel disease, rheumatoid arthritis, psoriasis, lupus erythematosus, diabetes, mycosis fungoides, or scleroderma.

51.-73. (canceled)
